(19) United States
(12) Patent Application Publication
(10) Pub. No.: US 2003/0032788 A1
(43) Pub. Date: Feb. 13, 2003

(54) Methods of inhibiting alcohol consumption

(75) Inventors: Eric Garver, Downingtown, PA (US); Guang-Chou Tu, East Norriton, PA (US); Yedy Israel, Philadelphia, PA (US)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: Thomas Jefferson University, Philadelphia, PA (US)

(21) Appl. No.: 09/932,300

(22) Filed: Aug. 17, 2001

Related U.S. Application Data
(63) Continuation-in-part of application No. 09/109,663, filed on Jul. 2, 1998, now Pat. No. 6,277,981.
(60) Provisional application No. 60/051,705, filed on Jul. 3, 1997.

(51) Int. Cl. C07H 21/04; A61K 48/00

(52) U.S. Cl. 536/23.2; 514/44

(57) ABSTRACT

The invention includes methods for reducing alcohol consumption in mammals by inhibiting expression of an aldehyde dehydrogenase (e.g., liver mitochondrial aldehyde dehydrogenase) in cells of the mammal. Expression of the aldehyde dehydrogenase can be inhibited by administering (locally or systemically) an antisense oligonucleotide (e.g., one 12-2000 residues in length) to the mammal, whereby expression of a relatively active allele of an aldehyde dehydrogenase gene can be inhibited. Aldehyde dehydrogenase activity can also be inhibited in the cells of a mammal, for the same purposes, by administering to the cells an expression vector encoding an inactive allele dominant of the aldehyde dehydrogenase. Upon expression, subunits of the protein encoded by the inactive allele can coalesce with one another or with subunits of the cells normally-expressed aldehyde dehydrogenase to lower the level of aldehyde dehydrogenase activity in the cell. The invention also relates to methods for predicting whether an antisense oligonucleotide (ASO) will be efficacious for inhibiting expression of a gene.

### CROSS REFERENCE TO RELATED APPLICATIONS

This application is a continuation-in-part of U.S. Application No. 09/109,663, filed Jul. 2, 1998, which is now allowed. This application is also entitled to priority, pursuant to 35 U.S.C. §119(e), to U.S. Provisional Application No. 60/051,705, filed Jul. 3, 1997.

### STATEMENT REGARDING FEDERALLY SUPPORTED RESEARCH AND DEVELOPMENT

This work was supported in part by grants from the National Institute on Alcohol Abuse and Alcoholism (NIH/NIAAA Grants Nos. R01-10967, R50AA107186, T32 AA 0763, and R37 AA 10630) and the U.S. Government may therefore have certain rights in this invention.

### REFERENCE TO A MICROFICHE APPENDIX

Not Applicable.

### BACKGROUND OF THE INVENTION

The field of the invention is treatment of alcoholism and alcohol abuse. Alcohol-(i.e., ethanol-)containing beverages and foodstuffs are consumed by a broad cross-section of the adult population. A significant fraction of alcohol consumers experience a problem with alcoholism or alcohol abuse at some period. For some, abstinence and counseling programs can help to resolve such problems. However, other individuals are able to resolve alcohol consumption problems only with great difficulty, or are not able to resolve them voluntarily. Such individuals can benefit from medical or pharmacological intervention to wean them from alcohol dependence and craving. A limited number of pharmacological agents are known which can assist an individual to refrain from alcohol consumption.

Acetaldehyde is an intermediary in the oxidation of alcohol by the body. When acetaldehyde metabolism is inhibited, acetaldehyde accumulates in the bloodstream, resulting in occurrence of toxic symptoms and great discomfort. An enzyme that has a major role in detoxification of acetaldehyde in the body is the liver mitochondrial acetaldehyde dehydrogenase (ALDH2; Schuckit, 2000, Am. J. Addict. 9:103-112). A dominant mutation in the ALDH2 gene, designated ALDH2-2, lowers or abolishes the activity of ALDH2 (Yoshida et al., 1985, Biochem. Genet. 23:585-590; Crabb et al., 1989, J. Clin. Invest. 83:314-316).

A protective genetic influence is associated with occurrence in an individual of the ALDH2-2 allele (Yoshida et al., 1985, Alcohol 2:103-106; Yoshida et al., 1985, Biochem. Genet. 23:585-590). Overall, the protective effect of the ALDH2-2 allele against alcohol abuse and alcoholism ranges from 66-90% for heterozygotes to 100% for homozygotes (Goedde et al., 1983, Isozymes: Curr. Top. Biol. Med. Res. 8:175-193; Harada et al., 1982, Lancet 2:827; Higuchi, 1994, Alcohol Alcohol. Suppl. 2:29-34; Thomasson et al., 1991, Am. J. Hum. Genet. 48:677-681; Tu et al., 1995, Behav. Genet. 25:59-65).

Individuals in whom the ALDH2-2 allele occurs do not efficiently oxidize acetaldehyde, and accumulation of toxic acetaldehyde results in a dysphoria characterized by dizziness, nausea, hypotension, and palpitations (Mizoi et al., 1983, Pharmacol. Biochem. Behav. 18:127-133). Therefore, interference with ALDH2 activity can decrease a human's alcohol tolerance for and desire to consume alcohol. Pharmacological compounds that interfere with acetaldehyde metabolism can be used to induce alcohol aversion in humans.

Disulfiram, for example, is a compound which interferes with metabolism of acetaldehyde in vivo. Alcohol consumption within 12 hours of disulfiram administration can produce facial flushing within 5 to 15 minutes, intense dilation of blood vessels in the face and neck, suffusion of the conjunctivae, throbbing headache, tachycardia, hyperpnea, and sweating. Nausea and vomiting can follow in 30 to 60 minutes, and may lead to hypotension, dizziness, and sometimes fainting and collapse. Discomfort attributable to post-disulfiram alcohol consumption can be so intense that patients are inhibited from imbibing alcohol. A significant number of patients are unable to use disulfiram, owing to adverse effects attributed to the drug, inability to maintain compliance with the required frequent dosing regimen, or ineffectiveness of the compound. U.S. Pat. No. 5,866,028 describes several other compounds asserted to be useful for inhibiting ALDH activity. None of the compounds disclosed in that patent have been widely clinically accepted. Thus, there is a need for alternative therapeutic agents which can inhibit alcohol consumption by interfering with acetaldehyde metabolism. The present invention satisfies this need.

Antisense oligonucleotides (ASOs) are short, usually synthetic, nucleic acids designed to bind to mRNA or other nucleic acids comprising specific sequences, taking advantage of Watson-Crick-type base pairing. Prior art ASO therapeutic strategies are designed to suppress the expression of specific genes involved in cancer, inflammatory diseases, and viral infections (Crooke et al., 1996, Annu. Rev. Pharmacol. Toxicol. 36:107-129). More than ten ASOs are currently undergoing human clinical trials for the treatment of various diseases (Matteucci et al., 1996, Nature 384(Supp.):20-22; Agrawal, 1996, Trends Biotechnol. 14: 376-387).

Antisense therapy comprising binding of an ASO to mRNA in a cell affected by a disease or disorder has, to date, been a therapeutic strategy wherein it has been difficult to identify efficacious target sites for a given RNA sequence (Gewirtz et al., 1996, Proc. Natl. Acad. Sci. U.S.A. 93:3161-3163). A significant shortcoming of prior art antisense strategies is the inability to accurately predict which ASOs will prove efficacious among a population of potentially efficacious ASOs (Laptev et al., 1994, Biochemistry 33:11033-11039). Because prior art attempts to predict the therapeutic efficacy of ASOs have been largely unsuccessful, selection of ASO sequences for antisense therapy has, prior to the present disclosure, been performed by empirically screening large numbers of potential antisense agents (Bennett et al., 1994, J. Immunol. 152:3530-3540). Using trial-and-error ASO selection strategies of the prior art, a large number of ASOs must be tested in order to discover a few sequences which exhibit significant efficacy as therapeutic ASOs. Prior art strategies require the screening of large numbers of ASOs because any portion of an mRNA molecule can be used to design a complementary ASO.

For example, an mRNA molecule which consists of 2000 nucleotide residues affords 1980 potential target sites for an ASO comprising twenty-one nucleotides which is complementary to twenty-one sequential nucleotide residues of the mRNA molecule. The trial-and-error methods of the prior art ASO selection process therefore recommend the manufacture and assay of at least 30-40 potential ASOs in order to identify likely no more than a few efficacious ASOs. Clearly, a method of designing ASOs which reduces or avoids dependence on trial-and-error selection methods would be of great value by reducing the duration and expense of ASO development efforts.

Investigations have been made by others to determine the effect upon efficacy of designing ASOs complementary to various regions of mRNA molecules. In general, these investigations have concentrated on complementation of an ASO to a discrete region within mRNA molecules. For example, various investigators have determined that efficacious ASOs may be constructed which are complementary:

a) to regions encompassing the 5′-cap site of an mRNA molecule (Ojala et al., 1997, Antisense Nucl. Drug Dev. 7:31-38),

b) to regions encompassing the transcription start site (Monia et al., 1992, J. Biol. Chem. 267:19954-19962),

c) to regions encompassing the translation initiation codon (Dean et al., 1994, Proc.

Natl. Acad. Sci. U.S.A. 91:11762-11766),

d) to regions encompassing the translation stop codon (Wang et al., 1995, Proc. Natl. Acad. Sci. USA 92:3318-3322),

e) to regions encompassing sites at which mRNA molecules are spliced (Agrawal et al., 1988, Proc. Natl. Acad. Sci. U.S.A. 86:7790-7794; Colige et al., 1993, Biochem. 32:7-11),

f) to regions encompassing the 5′-untranslated region of mRNA molecules (Duff et al., 1995, J. Biol. Chem. 270:7161-7166; Yamagami et al., 1996, Blood 87:2878-2884),

g) to regions encompassing the 3′-untranslated region of mRNA molecules (Bennett et al., 1994, J. Immunol. 152:3530-3540; Dean et al., 1994, J. Biol. Chem. 269:16146-16424), and

h) to regions encompassing the coding region (Laptev et al., 1994, Biochem. 33:11033-11039; Yamagami et al., 1996, Blood 87:2878-2884).

Because efficacious ASOs can, as demonstrated by these investigators, be complementary to any region of an mRNA molecule, the ASO designer is not provided any meaningful guidance by these studies.

Several strategies have been proposed to facilitate and simplify the selection process for efficacious ASOs. One strategy relies upon predictions of the binding energy between an ASO and a complementary sequence in an mRNA molecule. Chiang et al. (1991, J. Biol. Chem. 266:18162-18171) designed ten ASOs complementary to mRNA encoding human ICAM-1 protein with the aid of the computer program, OLIGO. These ten oligonucleotides were designed to maximize the melting temperature (Tm) of the oligonucleotide-mRNA complex. However, these investigators discovered that the efficacy of the ASOs as inhibitors of ICAM-1 expression did not correlate directly with either the Tm of the oligonucleotide-mRNA complex or the delta G°₃₇ (change in free energy upon association/dissociation of the oligonucleotide and the mRNA complex, as assessed at 37° C.). The most potent oligonucleotide (ISIS 1939) identified by these investigators exhibited a Tm value that was lower than those corresponding to the majority of the other oligonucleotides which were tested. Thus, maximization of binding energy between an ASO and a complementary mRNA is not sufficient to ensure therapeutic efficacy of the oligonucleotide.

Stull et al. (1992, Nucl. Acids Res. 20:3501-3508) investigated a systematic approach for predicting appropriate sequences within an mRNA molecule against which complementary ASOs could be constructed, by calculating three thermodynamic indices: (i) a secondary structure score (Sscore), (ii) a duplex score (Dscore); and (iii) a competition score (Cscore), which is the difference between the Dscore and the Sscore. The Sscore estimates the strength of local mRNA secondary structures at the MRNA binding site for the ASO. The Dscore estimates delta G_{formation}, the change in Gibbs free energy upon formation of the duplex, of the oligonucleotide-mRNA target sequence duplex. These three indices were compared to the efficacy of ASOs for inhibiting protein expression. It was found that the Dscore was the most consistent predictor of ASO efficacy in four of the five studies (the correlation factor r² ranged from 0.44 to 0.99 in these four studies). The results of the fifth study could not be predicted by any thermodynamic or physical index.

A second strategy for selecting efficacious ASOs is based upon predicting the secondary structure of mRNA. Wickstrom and colleagues (1991, In Prospects for antigense nucleic acid therapy of cancer and AIDS, Wickstrom, ed., Wiley-Liss, Inc., New York, 7-24) attempted to correlate the efficacy of potential ASOs with the secondary structure of the complementary region of the mRNA. It was hypothesized that ASOs would be the most efficacious when they were designed to be complementary to the target sequences within the mRNA molecule which were the least involved in the secondary and tertiary structure of the mRNA molecule. These investigators designed fourteen ASOs which were complementary to the predicted stems, loops, and bulges of human C-myc p65 MRNA. ASOs were designed which were complementary to regions of the mRNA molecule between the 5′-cap site and the translation initiation codon AUG, and included oligonucleotides which were complementary to sequences located within a predicted hairpin sequence which was located immediately 3′ to the AUG initiation codon. These investigators discovered that two fragments, one comprising the 5′-cap sequence and the other comprising a sequence located slightly 3′ relative to the cap sequence, were better target sequences for ASOs than the sequence spanning the AUG initiation codon, even though the sequence spanning the AUG initiation codon was located at an even weaker bulge and stem area.

Lima et al. (1992, Biochem. 31:12055-12061) designed six ASOs, each of which was complementary to a portion of a 47-nucleotide region that was able to achieve a stable hairpin conformation within an activated Ha-ras gene transcript. These investigators discovered that two of the oligonucleotides which were complementary to the loop portion of the hairpin structure had nearly equal binding affinity for the transcript. In contrast, they observed that oligonucleotides which were complementary to the double-stranded stem portion of the hairpin structure were less tightly bound, having affinity constants that were smaller by a factor of between 10⁵ and 10⁶. These results suggest that mRNA sequences which lie within regions of secondary structure may be undesirable target sequences for designing complementary ASO.

Thierry et al. (1993, Biochem. Biophys. Res. Commun. 190:952-960) compared the efficacy of ASOs which were complementary to either the 5′-end of the coding region of or to a single-stranded loop in the MRNA encoded by the multi-drug resistance gene mdrl. The results obtained by these investigators indicate that the oligonucleotides targeted to the single-stranded loop were more efficacious and specific than the oligonucleotides targeted to the 5′-end coding region. However, Laptev et al. (1994, Biochem. 33:11033-11039) obtained results which were not consistent with that suggestion. Laptev et al. concluded that the most efficacious ASOs were those which were complementary to mRNA sequences that were predicted to form clustered double-stranded secondary structures.

Still other investigators presented evidence that the most efficacious ASOs (ISIS 1939 and ISIS 2302) were those which were complementary to regions of human ICAM-1 mRNA, which regions were predicted by computer modeling to form stable stem-loop structures (Chiang et al., 1991, J. Biol. Chem. 266:18162-18171; Bennett et al., 1994, J. Immunol. 152:3530-3540). Oligonucleotides which were complementary to mRNA sequences upstream or downstream from these putative stem-loop structures had significantly less inhibitory activity (Bennett et al., 1994 Adv. Pharmacol. 28:1).

Fenster et al. (1994, Biochemistry 33, 8391-8398) observed that inhibition of gene expression by an ASO was highly dependent upon the position of the MRNA sequence to which the oligonucleotide was complementary. These investigators discovered that the most potent ASOs to effect inhibition of the Rev-response element of the human immunodeficiency virus (HIV) were complementary to MRNA target sites corresponding to the stem-loop V region of the HIV mRNA. This region of the HIV mRNA is known to be important for full and efficient Rev-response element function. ASOs targeted to other, non-critical Rev-response element stem-loops (e.g. SLI and SLIII) were determined by these investigators to be either non-efficacious or 30-fold less efficacious than stem-loop V oligonucleotides for inhibiting Rev-response element function.

Hence, it is clear that the bulk of ASO selection strategies reported in the prior art have been directed to designing ASOs which are complementary to discrete regions within mRNA molecules, rather than to particular sequences within mRNA.

Recently, Ho et al. (1996, Nucl. Acids Res. 24:1901-1907; 1998, Nature Biotechnol. 16:59-63) developed a novel approach to rationally select ASOs. These investigators contacted an mRNA molecule encoding human multi-drug resistance- 1 protein and an mRNA molecule encoding angiotensin type I receptor protein with a library of chimeric oligonucleotides. Hybridized mRNA was subsequently treated with RNase H, an enzyme which catalyzes the hydrolytic cleavage of only the RNA strand of an RNA-DNA duplex. The RNA fragments which were generated were sequenced to identify regions on the mRNA sequence which were involved in RNA-DNA duplex formation. Using the sequence information, these investigators constructed ASOs which were complementary to these regions and found those particular ASOs to be significantly more efficacious than randomly-selected oligonucleotides for inhibiting human multi-drug resistance-I protein or angiotensin type I receptor protein expression. These results demonstrate that it is feasible to construct improved ASOs by incorporating therein sequences which are complementary to particular nucleotide sequences found in mRNA molecules.

Skilled workers in the art have concluded the therapeutic efficacy of an ASO which is complementary to a particular target sequence within an mRNA molecule has not heretofore been accurately predictable (Gewirtz et al., 1996, Proc. Natl. Acad. Sci. U.S.A. 93:3161-3163). Because efficacious ASOs have been made which are complementary to most or all regions of mRNA molecules, the ASO designer cannot be meaningfully guided by selection of any particular mRNA region. Methods for predicting the efficacy of ASOs by maximization of Tₘ or delta G_{formation} have not consistently yielded correct predictions, and thus are similarly of limited use to the ASO designer. Analyses of the secondary structure of an mRNA do not clearly identify potential ASO-binding sites. Library-based RNaseH degradation studies are laborious and complex. Other skilled workers in the art have recognized that a long-felt, but unmet, need exists for methods of selecting the most potent target sequences within a given mRNA sequence (Szoka, 1997, Nature Biotechnol. 15:509).

In April of 1998, results of a consensus reached at U.S. National Institutes of Health conference on this subject were reported. These results included the conclusions that “{i}t appears that the only way to generate an active oligomer is by brute force” and that “{o}ptimally it is best to screen 30-40 oligos to obtain one species that is maximally active, but this may be impossible because of time and cost considerations” (Stein, 1998, Antisense and Nucleic Acid Drug Development 8: 129-132).

Taken together, the results of these prior art methods for designing an ASO sequence offer little guidance to the ASO designer. The present invention overcomes the shortcomings of prior art ASO design methods by providing a method for designing efficacious ASOs.

### BRIEF SUMMARY OF THE INVENTION

The invention relates to antisense oligonucleotides (ASOs) for inhibiting expression of an aldehyde dehydrogenase gene (e.g., the ALDH2 gene of a human or other animal) in a cell. The oligonucleotide comprises at least 12 nucleotide residues (e.g., 12-50, 14-30, or 16-23 residues), and has a sequence selected or designed such that the oligonucleotide anneals with a portion of an RNA molecule corresponding to the aldehyde dehydrogenase gene (e.g., an mRNA or a primary transcript of the gene). The portion comprises a GGGA motif. When the oligonucleotide is present in the cell, expression of the aldehyde dehydrogenase gene is inhibited, in that the aldehyde dehydrogenase activity attributable to the enzyme encoded by the gene is lower than it would otherwise be in the absence of the oligonucleotide. By way of example, expression of the ALDH2 gene can be inhibited using an oligonucleotide having a sequence which consists of, or at least comprises, one of SEQ ID NOs: 98, 107, 109, and 111. SEQ ID NO: 107 is nucleotide residues 241 to 261 of the rat ALDH2 MRNA sequence (SEQ ID NO: 108) disclosed in Farres et al. (1989, Eur. J. Biochem. 180:67-74; GENBANK® accession no. NM_{—}032416), and SEQ ID NO: 98 is its complement. SEQ ID NO: 109 is nucleotide residues 237 to 257 of the human ALDH2 mRNA sequence (SEQ ID NO: 110) disclosed in GENBANK® accession no. NM_{—}000690, and SEQ ID NO: 111 is its complement.

The invention also includes pharmaceutical compositions which comprise oligonucleotides for inhibiting an aldehyde dehydrogenase, the oligonucleotide being suspended in (or otherwise admixed with) a pharmaceutically acceptable carrier. As an alternative, the pharmaceutical can comprise a transcription vector instead of the oligonucleotide. The transcription vector comprises a region that encodes an antisense oligonucleotide for inhibiting expression of an aldehyde dehydrogenase gene operably linked with promoter/regulatory sequences necessary to effect production of the oligonucleotide in a cell. The antisense oligonucleotide can, for example, have a length of about 12 to 1989 nucleotide residues, and is not limited to relatively short (e.g., 12-50 residue) polynucleotides.

In another aspect, the invention includes a method that can be used to decrease ethanol tolerance in a human and decrease the desire of the human to consume ethanol. The method comprises administering the aldehyde dehydrogenase-directed ASO described above to liver cells of the human. The oligonucleotide inhibits expression of the aldehyde dehydrogenase gene in the cells, inhibits the ability of the human to metabolize acetaldehyde, decreases ethanol tolerance in the human, and decrease the desire of a human to consume ethanol. The ASO can be administered in one of the pharmaceutical compositions described herein, and can be administered systemically or directly to liver tissue.

The invention also relates to a polynucleotide that can be used to inhibit aldehyde dehydrogenase activity in a cell (e.g., in a human liver cell). The polynucleotide encodes an exogenous (i.e., relative to the cell) ALDH2-2 allele operably linked with a promoter/regulatory region. The ALDH2-2 allele is expressed in the cell when the polynucleotide is delivered to the interior of the cell. When an ALDH2-2 monomer is included in an aldehyde dehydrogenase tetramer that does not normally include an ALDH2-2 monomer, the activity of the tetramer is decreased. Thus, when an exogenous ALDH2-2 monomer is expressed in a cell and it multimerizes with the cell's normal aldehyde dehydrogenase monomers, an aldehyde dehydrogenase tetramer having lower activity (relative to the same cell in which the ALDH2-2 monomer is not expressed) is formed, and aldehyde dehydrogenase activity is inhibited in the cell. The polynucleotide can be part of an expression vector, and the polynucleotide or vector can be included in a pharmaceutical composition. The ALDH2-2-encoding polynucleotide can be used to decrease ethanol tolerance, inhibit ethanol intake, and decrease the desire for ethanol consumption in a human. These methods are effected by administering the polynucleotide (or an expression vector comprising it) to liver cells of a human. By way of example the ALDH2-2-encoding polynucleotide can encode the protein encoded by the mRNA disclosed in GENBANK® accession no. NM_{—}000690 (which encodes the normal human ALDH2 subunit), wherein the guanine residue that occurs at residue 1543 is replaced by an adenine residue, yielding a dominant negative subunit.

The invention also relates to a method of making an ASO for inhibiting expression of an aldehyde dehydrogenase gene that is expressed in a cell of an animal. The method comprises selecting a portion of an RNA molecule corresponding to the gene. The portion comprises a GGGA motif. The ASO is at least 12 nucleotide residues (e.g., 12-50, 14-30, or 16-23 residues) in length and is complementary to the portion. The ASO inhibits expression of the gene when the ASO is administered to the interior of the cell.

In another aspect, the invention relates to an antisense oligonucleotide for inhibiting expression of a gene which encodes TNF-alpha in an animal. The oligonucleotide comprises from 12 to 50 nucleotide residues. At least 90% of the nucleotide residues of this oligonucleotide are complementary to a region of an RNA molecule which corresponds to the gene, and the region comprises a GGGA motif. In one embodiment, the oligonucleotide comprises from 14 to 30 nucleotide residues, comprises a TCCC motif, and at least 95% of the nucleotide residues of the oligonucleotide are complementary to the region. In another embodiment, the oligonucleotide comprises from 16 to 21 nucleotide residues, comprises a TCCC motif, and is completely complementary to the region.

The invention also relates to a method of making an antisense oligonucleotide for inhibiting expression of a gene in an animal. This method comprises identifying an RNA molecule corresponding to the gene, wherein the RNA molecule comprises a GGGA motif, and synthesizing an oligonucleotide complementary to at least a portion of the RNA molecule. The portion comprises the GGGA motif. In other embodiments of this method, at least a portion of the oligonucleotide comprises either a randomly-generated sequence or a sequence that is complementary to the targeted gene. In another embodiment, gene is a human gene. In still another embodiment, the RNA molecule is the primary transcript of the gene.

The invention includes an antisense oligonucleotide made by this method.

The invention further relates to a method of treating an animal afflicted with a disease or disorder characterized by the presence in an affected cell of the animal of an RNA molecule which corresponds to a gene and comprises a region comprising a GGGA motif. This method comprises providing an antisense oligonucleotide which is at least 90% complementary to the region and administering the oligonucleotide to the animal. In one aspect of this method, the antisense oligonucleotide is at least 95% complementary to the region. In a preferred embodiment, the antisense oligonucleotide is completely complementary to the region. In yet another embodiment, the RNA molecule is the primary transcript of the gene. In another aspect of this method, at least one linkage between nucleotide residues of the oligonucleotide is a phosphorothioate linkage.

The invention also relates to a method of inhibiting expression of a gene in an animal cell. This method comprises administering to the cell an antisense oligonucleotide which is complementary to a region of an RNA molecule corresponding to the gene, wherein the region comprises a GGGA motif.

The invention includes a method of predicting the efficacy of an antisense oligonucleotide for inhibiting expression of a gene. This method comprises determining whether the antisense oligonucleotide is complementary to a region of an RNA molecule corresponding to the gene, wherein the region comprises a GGGA motif. If so, this is an indication that the antisense oligonucleotide is efficacious for inhibiting expression of the gene.

The invention further relates to a method of separating from a mixture of oligonucleotides an antisense oligonucleotide which is efficacious for inhibiting expression of a gene. This method comprises contacting the mixture with a support linked to an oligonucleotide comprising a GGGA motif, whereby the efficacious antisense oligonucleotide associates with the support, and separating the support from the mixture.

In another embodiment, the invention includes a method of administering an oligonucleotide comprising a GGGA motif (i.e., one of those described herein) with a delivery molecule such as a dendrimer in order to form a delivery complex. Administration of the delivery complex to a cell leads to uptake of the delivery complex by the cell and internalization of the oligonucleotide. In one embodiment, the oligonucleotide and delivery molecule are linked by a chemical bond that is cleaved intracellularly. By way of example, the delivery molecule can be a dendrimer (see, e.g., Merino et al., 2001, Chemistry 7:3095-3105).

### BRIEF DESCRIPTION OF THE SEVERAL VIEWS OF THE DRAWINGS

The foregoing summary, as well as the following detailed description of the invention, will be better understood when read in conjunction with the appended drawings. The invention is not limited to the precise arrangements and instrumentalities shown.

FIG. 1 is a bar graph which depicts the effect of three different ASOs on the expression of TNF-alpha by cultured Kupffer cells. Data are reported as a percentage of TNF-alpha protein expression in control cultures which were not treated with an ASO.

FIG. 2 is a bar graph which indicates the inhibition of TNF-alpha expression achieved by culturing cells in the presence of the indicated ASOs.

FIG. 3 is an image which portrays the results of Northern hybridization experiments described herein in Example 4. “Motif containing” refers to whether the ASO used in the corresponding lane comprised a TCCC motif.

FIG. 4 is the nucleotide structure of the human TNF-alpha gene. Nucleotide residues corresponding to GGGA motifs in transcription products encoding TNF-alpha are indicated with capital letters. Nucleotide residues corresponding to regions of a transcription product which could be used as target sequences for design of efficacious ASO having a length of up to 21 nucleotide residues are underlined.

FIG. 5 comprises FIGS. 5A, 5B, 5C, and 5D. Each part of FIG. 5 shows an image of agarose gel electrophoresis results of the quantitation of steady-state mRNA by competitive RT-PCR. Panels 5A and 5B show ALDH2 mRNA expression and Panels 5C and 5D show GDH mRNA expresion.

FIG. 6 is a graph representing the effect of ASO-9 on ethanol consumption following an 18-hour water deprivation period.

FIG. 7 comprises FIGS. 7A and 7B. FIG. 7A is a graph which illustrates the inhibitory effect on ethanol consumption of ASO-9 (24 milligram/kilogram/day). FIG. 7B is a graph which illustrates the inhibitory effect on ethanol consumption of disulfiram administration (DS; 100 milligram/kilogram/day).

FIG. 8 comprises FIGS. 8A and 8B. FIG. 8A is a schematic depiction of the LNCX vector, comprising the human ALDH2-1 cDNA (pLNCE). FIG. 8B represents the LNCX vector, comprising the human ALDH2-2 cDNA (PLHCK 3′ UT).

FIG. 10 is a bar graph depicting the percentage of H4 cell clones out of the total number of clones measured for each group (vector alone, ALDH2-1 transduced, ALDH2-2 transduced) that exhibited particular ALDH2 activity levels.

FIG. 11 is the sequence (SEQ ID NO: 108) disclosed in Farres et al. (1989, Eur. J. Biochem. 180:67-74) for the mRNA encoding rat mitochondrial aldehyde dehydrogenase 2 (GENBANK® accession no. NM_{—}032416). The underlined residues are residues 241-261 and have the sequence SEQ ID NO: 107.

FIG. 12 is the sequence (SEQ ID NO: 110) for the mRNA encoding human mitochondrial aldehyde dehydrogenase 2 (GENBANK® accession no. NM_{—}000690). The underlined residues are residues 237-257, have the sequence SEQ ID NO: 109, and exhibit high homology with SEQ ID NO: 107.

### DETAILED DESCRIPTION

The invention relates to methods of treating or alleviating alcoholism and alcohol abuse by inhibiting expression of the ALDH2 gene. Inhibiting expression of the ALDH2 gene can be effected using the methods disclosed herein. Previously known inhibitors of ALDH activity (e.g., disulfiram) did not specifically inhibit ALDH2 activity, and their effect on ALDH activity was not limited to the liver; disulfiram, for example, also inhibits brain ALDH2. Although it was previously known that the liver is a site of metabolism for acetaldehyde resulting from ethanol consumption, it was not known whether inhibition of liver mitochondrial ALDH alone would be sufficient to induce an ethanol-aversive effect. Antisense oligonucleotides do not cross the blood-brain barrier. It has been discovered that using an antisense oligonucleotide to specifically inhibit liver and peripheral mitochondrial ALDH2 expression is sufficient to induce accumulation of acetaldehyde in a mammal (e.g., a human or rat) following ingestion of alcohol by the mammal. Furthermore, it has been found that this acetaldehyde accumulation is sufficient to induce unpleasant side effects (e.g., flushing and cramping) in mammals in whom ALDH2 expression is inhibited, which can increase reluctance of the mammal to consume additional ethanol.

ASOs were designed and found to be efficacious for inhibiting expression of aldehyde dehydrogenase (i.e., ALDH2) in liver cells and for inhibiting ethanol consumption in rats. In one aspect, an efficacious ASO can be designed based on occurrence of a GGGA motif in a portion of an RNA molecule corresponding to the aldehyde dehydrogenase gene with which the ASO is complementary. Administration of one ASO (designated ASO-9) to rats resulted in inhibition of liver aldehyde dehydrogenase activity in the rats. Presumably due to the reduced ability of their livers to further metabolize acetaldehyde formed following ingestion of ethanol, ASO-9-treated rats exhibited an aversion to ethanol. These results demonstrate that anti-ALDH ASOs described herein can be used to inhibit ethanol intake in alcoholics and other abusers of alcohol.

The invention is not limited to designing ASOs that are effective for inhibiting ethanol consumption. The invention also relates to the discovery of a more general method of designing efficacious ASOs for use in antisense nucleic acid methods including methods of inhibiting gene expression.

This discovery was made during a study wherein a large number of phosphorothioate-modified ASOs, each comprising from nineteen to twenty-one nucleotide residues, were designed to be complementary to various regions of an RNA molecule which encodes rat TNF-alpha protein. After screening the ASOs for their ability to inhibit expression of TNF-alpha, it was observed that only an ASO which was complementary to a fragment in the 3′-untranslated region of the mRNA markedly inhibited (i.e., >90% inhibition) the expression of TNF-alpha protein by cultured rat Kupffer cells. The gene which specifies this mRNA fragment has been reported (GENBANK® DDBJ D00475; NCBI Seq. Id. #220920). The nucleotide sequence of this gene comprises twenty-eight tetranucleotide 5′-GGGA-3′ sequences (hereinafter referred to as “GGGA motifs”). Accordingly, ASOs which were complementary to this sequence had nucleotide sequences which comprised at least one copy of the sequence 5′-TCCC-3′ (hereinafter referred to as a “TCCC” motif).

A series of ASOs, each comprising between sixteen and twenty-one nucleotide residues, were designed, synthesized, and screened to determine the efficacy thereof for inhibiting expression of TNF-alpha protein. It was discovered that most ASOs which were complementary to at least one of the twenty-eight TNF-alpha GGGA motifs (i.e., any ASO having a nucleotide sequence comprising at least one TCCC motif) displayed high inhibitory efficacy.

It was further discovered that the presence of the TCCC motif in an ASO is an indication that the ASO is efficacious for inhibiting protein expression from genes unrelated to TNF-alpha.

Because the presence of the GGGA motif in an RNA molecule has not previously been identified as a basis for designing efficacious ASOs, the existence of known efficacious ASOs having sequences comprising a TCCC motif was investigated. The results of a comprehensive search indicated that about half of the most efficacious ASOs which have been reported comprise the TCCC motif. Recognition of the significance of the TCCC motif in efficacious ASOs represents a significant advance over the prior art. The presence of the TCCC motif in an ASO complementary to an RNA molecule is an indication that the ASO will inhibit expression of the protein encoded by the RNA molecule. Thus, the skilled worker presented with either the nucleotide sequence of an RNA molecule or the sequence of a gene encoding an RNA molecule is enabled to design an ASO which will efficaciously inhibit expression of the RNA molecule or gene by designing the ASO to be complementary to that portion of the RNA molecule which comprises a GGGA motif.

Definitions

As used herein, the term “flanking” is used to refer to nucleotide sequences which are directly attached to one another, having no intervening nucleotide residues. By way of example, the pentanucleotide 5′-AAAAA-3′ is flanking the trinucleotide 5′-TTT-3′ when the two are connected thus: 5′-AAAAATTT-3′ or 5′ -TTTAAAAA-3′, but not when the two are connected thus: 5′-AAAAACTTT-3′. In the latter case, the C residue is said to be “interposed” between the pentanucleotide and the trinucleotide.

As used herein, the term “affected cell” refers to a cell in an animal afflicted with a disease or disorder, which affected cell has an altered phenotype relative to a cell of the same type in an animal not afflicted with the disease or disorder.

As used herein, the term “oligonucleotide” means a nucleic acid-containing polymer, such as a DNA polymer, an RNA polymer, or a polymer comprising both deoxyribonucleotide residues and ribonucleotide residues. This term further includes other polymers, such as polymers comprising modified or non-naturally-occurring nucleic acid residues and polymers comprising peptide nucleic acids. Each of these types of polymers, as well as numerous variants, are known in the art. This term includes, without limitation, both polymers which consist of nucleotide residues, polymers which consist of modified or non-naturally-occurring nucleic acid residues, and polymers which consist of peptide nucleic acid residues, as well as polymers comprising these residues associated with a support or with a targeting molecule, such as a cell surface receptor-binding protein.

As used herein, the term “antisense oligonucleotide” (“ASO”) means a nucleic acid polymer, at least a portion of which is complementary to a nucleic acid which is present in a normal cell or in an affected cell. In one embodiment, the ASOs of the invention are relatively short, and preferably comprise from twelve to about fifty nucleotide residues. More preferably, the ASOs comprise from fourteen to about thirty nucleotide residues. Most preferably, the ASOs comprise from sixteen to twenty-one nucleotide residues. In another embodiment, the ASOs are relatively long (e.g., 50-2000 residues in length). The ASOs can cover a few hundred residues, up to the total length of the MRNA or primary RNA transcript generated from the corresponding gene. The ASOs of the invention include, but are not limited to, phosphorothioate oligonucleotides and other modifications of oligonucleotides.

As used herein, the term “antisense agent” means an ASO suspended in a pharmaceutically acceptable carrier, whereby the ASO can be delivered to a cell of an animal, preferably a human. The term “antisense agent” includes naked DNA ASOs and naked RNA ASOs for delivery to a cell of an animal.

As used herein, the term “antisense therapy” means administration to an animal of an antisense agent for the purpose of alleviating a cause or a symptom of a disease or disorder with which the animal is afflicted.

As used herein, an oligonucleotide “associates” with another oligonucleotide or to a support to which the other oligonucleotide is linked when it binds to the other oligonucleotide in an affinity-dependent manner. By way of example, an oligonucleotide which hydrogen bonds to another oligonucleotide having a complementary nucleotide sequence when contacted therewith is said to associate with a support to which the other oligonucleotide is linked when the oligonucleotide is contacted with the medium.

As used herein, the term “binding energy” means the thermodynamic change in free energy which accompanies the binding of two complementary nucleic acids, one to the other. Binding energy is frequently expressed in terms of a change in the Gibbs free energy (delta G or delta G_{formation}) at a given temperature.

“Complementary” refers to the broad concept of sequence complementarity between regions of two nucleic acid strands or between two regions of the same nucleic acid strand. A first region of a nucleic acid is complementary to a second region of the same or a different nucleic acid if, when the two regions are arranged in an anti-parallel fashion, at least one nucleotide residue of the first region is capable of base pairing with a nucleotide residue of the second region. Preferably, when the first and second regions are arranged in an anti-parallel fashion, at least about 50%, and preferably at least about 75%, at least about 90%, or at least about 95% of the nucleotide residues of the first region are capable of base pairing with nucleotide residues in the second region. Most preferably, all nucleotide residues of the first region are capable of base pairing with nucleotide residues in the second region (i.e., the first region is “completely complementary” to the second region). It is known that an adenine residue of a first nucleic acid strand is capable of forming specific hydrogen bonds (“base pairing”) with a residue of a second nucleic acid strand which is anti-parallel to the first strand if the residue is thymine or uracil. Similarly, it is known that a cytosine residue of a first nucleic acid strand is capable of base pairing with a residue of a second nucleic acid strand which is anti-parallel to the first strand if the residue is guanine. It is understood that structure of nucleotide residues may be modified, whereby the complementation properties of the modified residue differs from the complementation properties of the naturally-occurring residue. Such modifications, and methods of effecting such modification, are known in the art.

As used herein, the term “complementary region of an RNA molecule” means a nucleotide sequence within an RNA molecule to which nucleotide sequence an ASO is complementary.

As used herein, an RNA molecule “corresponds” to a gene if the RNA molecule is generated upon transcription of the gene.

As used herein, an RNA molecule includes, without limitation, both the primary transcript (“pre-mRNA”) obtained by transcribing a gene and a messenger RNA (“mRNA”) obtained by transcribing a gene and processing the primary transcript.

As used herein, the term “gene” means a DNA sequence which, upon transcription thereof, yields an RNA molecule which encodes a protein and associated control sequences such as a translation initiation site, a translation stop site, a ribosome binding site, (optionally) introns, and the like. Alternately, the gene may be an RNA sequence which encodes a protein and associated control sequences such as a translation initiation site, a translation stop site, a ribosome binding site, and the like.

As used herein, the term “gene expression” includes both gene transcription, whereby DNA (or RNA in the case of some RNA-containing viruses) corresponding to a gene is transcribed to generate an RNA molecule and RNA translation, whereby an RNA molecule is translated to generate a protein encoded by the gene.

As used herein, the term “inhibition of gene expression” means inhibition of DNA transcription (or RNA transcription in the case of some RNA-containing viruses), inhibition of RNA translation, inhibition of RNA processing, or some combination of these.

As used herein, the term “oligonucleotide delivery agent” means a composition of matter which can be used to deliver an ASO to a cell in vitro or in vivo.

As used herein, the term “pharmaceutically-acceptable carrier” means a chemical composition with which an ASO of the invention may be combined and which, following the combination, can be used to administer the ASO of the invention to an animal.

As used herein, the term “protein expression” is used to refer both to gene expression comprising transcription of DNA (or RNA) to form an RNA molecule and subsequent processing and translation of the RNA molecule to form protein and to gene expression comprising translation of mRNA to form protein.

As used herein, the term “TNF-alpha-associated disease or disorder” means a disease or disorder of an animal which is caused by elevated TNF-alpha expression or a disease or disorder which results in elevated TNF-alpha expression, wherein elevated TNF-alpha expression is determined relative to an animal not afflicted with the disease or disorder.

As used herein, the term “TNF-alpha-specific ASO” means an ASO which comprises a TCCC motif and which is complementary to an RNA molecule encoding TNF-alpha.

As used herein, the term “TCCC motif” means a tetranucleotide portion of an ASO, having the sequence 5′-TCCC-3′. It is understood that each of the four nucleotide residues of the TCCC motif may be any chemical entity which exhibits substantially the same complementarity properties as the residue it substitutes. Thus, the term TCCC motif includes any chemical entity which is capable of binding with a GGGA motif with substantially the same complementarity properties as a tetranucleotide portion of an ASO, having the sequence 5′-TCCC-3′.

As used herein, the term “GGGA motif” means a portion of an RNA molecule comprising a tetranucleotide having the sequence 5′-GGGA-3′.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Description

The invention relates to methods of treating and alleviating alcoholism and alcohol abuse by inhibiting expression of the ALDH2 gene.

Previous attempts to treat alcoholism focused on the use of drugs that inhibit ALDH enzymatic activity. For example, disulfiram is a drug that inhibits ALDH and is approved in the U.S. for the treatment of alcoholism. The enzymatic inhibitory properties of disulfiram are not specific to ALDH2. Disulfiram alone only slightly inhibits ALDH2 activity and must be administered in a way that allows its metabolism into active compounds, to inhibit ALDH2 activity.

A major drawback of disulfiram and other chemical inhibitors of ALDH is lack of compliance by patients. These agents produce an array of unpleasant side effects such as sensory and motor neuropathies, optic neuritis, orthostatic hypotension and hypersensitivity reactions (Dupoy et al., 1995, Rev. Med. Interne. 16:67-72; Peachey et al., “Effectiveness of Aversion Therapy Using Disulfiram and Related Compounds,” in Human Metabolism of Alcohol, Vol. I, B.R. Crowe KE (ed.), pp. 158-167 (1989); Gallant, “The Use of Psychopharmacologic Medications in Alcoholism,” in A Guide to Diagnosis, Intervention and Treatment (W.W. Norton and Company, 1987); Hugues, et al., 1992, Rev. Med. Interne. 13:465-470; Chick, 1999, Drug Saf. 20:427-435).

In contrast to known treatments for alcoholism which involve administration of a drug that either directly or indirectly inhibits ALDH2 activity, a method of selectively inhibiting ALDH2 activity by inhibiting expression of the ALDH2 gene has been discovered. This inhibition is effected by administration of an ASO which anneals to a portion of an RNA molecule that corresponds to an ALDH2 transcript, thereby inhibiting expression of the gene (i.e., inhibiting formation of functional liver mitochondrial ALDH enzyme). The invention includes an ASO for inhibiting expression of an ALDH gene, such as a human liver mitochondrial aldehyde dehydrogenase (ALDH2) gene. The ASO is preferably designed so that it is complementary to a portion of an mRNA molecule having a GGGA motif therein. An example of such an ASO has the sequence SEQ ID NO: 98 (corresponding to the rat ALDH2 gene), and another example has the sequence SEQ ID NO: 111 (corresponding to the human ALDH2 gene). SEQ ID NO: 98 is TCCTCCTTGT TCCCTTCGGC T, and SEQ ID NO: 111 is TCTTCCTTGT CCCCTTCAGC T.

Treatment of Alcohol Abuse and Alcoholism

Following ethanol consumption, cells of the liver metabolize ethanol to form acetaldehyde. Acetaldehyde is toxic to the cells and is normally oxidized to form (relatively non-toxic) acetate. An enzyme that has a major role in detoxification of acetaldehyde is liver mitochondrial ALDH (ALDH2, as described in Schuckit, 2000, Am. J. Addict. 9:103-112). A dominant mutation in the ALDH2 gene, present in an allele of the gene designated ALDH2-2, lowers or abolishes the activity of this enzyme (Yoshida et al., 1985, Biochem. Genet. 23:585-590; Crabb et al., 1989, J. Clin. Invest. 83:314-316).

Research on the genetics of alcoholism indicates that a protective genetic influence is associated with the ALDH2-2 allele (Yoshida et al., 1985, Alcohol 2:103-106; Yoshida et al., 1985, Biochem. Genet. 23:585-590). Overall, the protective effect of the ALDH2-2 allele against alcohol abuse and alcoholism ranges from 66-90% for heterozygotes and to 100% for homozygotes (Goedde et al., 1983, Isozymes: Curr. Top. Biol. Med. Res. 8:175-193; Harada et al., 1982, Lancet 2:827; Higuchi, 1994, Alcohol Alcohol. Suppl. 2:29-34; Thomasson et al., 1991, Am. J. Hum. Genet. 48:677-681; Tu et al., 1995, Behav. Genet. 25:59-65). Individuals carrying the ALDH2-2 allele do not efficiently oxidize acetaldehyde, and accumulation of toxic acetaldehyde results in a dysphoria characterized by dizziness, nausea, hypotension, and palpitations. Reducing or inhibiting ALDH2 expression, thereby reducing levels of ALDH2 protein, can decrease a human's tolerance for and desire to consume alcohol.

The invention includes methods of decreasing a mammal's tolerance for ethanol and desire to consume by inhibiting expression of an aldehyde dehydrogenase gene (e.g., the liver mitochondrial ALDH2 gene). The method comprises administering to the mammal an ASO which comprises at least 12 nucleotide residues (e.g., 12-50, 14-30, or 16-23 residues) and which is complementary to a portion of an RNA molecule corresponding to the gene. The sequence of the ASO is selected such that it anneals (or is expected to anneal) with the portion in the environment of the cell (i.e., within the cell). The portion preferably comprises a GGGA motif, and the ASO is preferably completely complementary to the GGGA motif. The ASO is also complementary (although not necessarily completely complementary) to one or both regions that flank the GGGA motif. Overall, the complementarity of the ASO with the portion should be 90% or greater.

In an example disclosed herein, the ASO is designated ASO-9 and has the nucleotide sequence SEQ ID NO: 98, which is complementary to residues 241-261 of the sequence (SEQ ID NO: 108) of the rat mitochondrial ALDH2 mRNA disclosed by Farres et al. (1989, Eur. J. Biochem. 180:67-74). When the ASO is provided to the cell, it anneals with the RNA molecule if the ALDH gene is transcribed and inhibits expression of the gene (i.e., inhibits formation of functional ALDH enzyme). The level of ALDH activity in the cell increases by less than it would have if the ASO were not present when the gene is expressed.

The ALDH2 gene has a significant role in conversion of acetaldehyde generated following ethanol ingestion to acetate. Hence, inhibition of expression of the ALDH2 gene can be used to treat alcoholism and alcohol abuse. An analogous ASO that can be used to inhibit ALDH activity in human cells has the sequence SEQ ID NO: 111, and is complementary to residues 237-257 of the sequence (SEQ ID NO: 110) of the human mitochondrial ALDH2 mRNA disclosed in GENBANK® accession no. NM_{—}000690.

The route by which the ASOs are administered to the cells in which inhibition of ALDH gene expression is desired is not critical. Substantially any route or formulation which enables the ASO to reach the interior of the cells from the site of administration can be used. For example, a suspension (e.g., in saline or phosphate buffered saline) of the ASO can be administered systemically (e.g., by intravenous injection or infusion) and be carried by the blood stream to the cells. Alternatively, the ASO can be suspended in a preparation that is locally applied to a tissue comprising the cells (i.e., in a manner which does not cause most of the preparation to enter the blood stream shortly after administration). Furthermore, the ASO can be contained within a slowly-dissolving or other sustained-release preparation in order to extend the period during which the cells are contacted with the ASO. A variety of such pharmaceutically acceptable carriers are known in the art for use with ASOs, and substantially all of those known carriers can be used to administer the ASOs described herein, including dendrimers, fractured dendrimers, and fusogenic peptides.

Alternatively, the ASO can be administered in the form of a transcription vector for transcribing the ASO in the desired cells. The transcription vector the ASO operably linked with one or more promoter/regulatory sequences. Upon delivery to the interior of a cell, the normal transcriptional components of the cell are enabled by the promoter/regulatory sequence(s) to transcribe the ASO from the vector, resulting in intracellular production of the ASO. Where delivery of the ASO to particular cell or tissue types is desired, the promoter/regulatory sequence preferably includes a promoter that is preferentially used in cells or tissues of the desired type.

The invention includes another method of treating alcohol addiction and alcoholism by inhibiting ALDH activity in cells (e.g., in human liver cells). According to this method, an exogenous allele of an ALDH gene that exhibits less activity than an ALDH that is normally expressed by the cell is provided to the cell. Expression of the less active allele in the cell causes formation of monomers of the less active enzyme. Combination of the less active monomers with the endogenous ALDH monomers to form the usual tetrameric quaternary structure of ALDH results in formation of ALDH tetramers that exhibit less activity than endogenous ALDH tetramers.

By way of example, an allele designated ALDH2-2 of the human liver mitochondrial ALDH can be provided to liver cells of a human who does not normally express the ALDH2-2 allele. When the ALDH2-2 allele is expressed in the human's liver cells, the level of ALDH activity in the liver cells decreases, the ability of the human's liver to metabolize acetaldehyde decreases, and the human's tolerance for and desire to consume ethanol decrease as well. Thus, this method can be used to treat or inhibit alcoholism and alcohol abuse in humans.

The precise mechanism used to deliver the less active ALDH allele to the liver cells is not critical. Substantially any method of delivering an allele of a gene to a cell in an expressible form can be used. By way of example, numerous techniques which are generally referred to as ‘gene therapy’ are known, by which an expressible gene can be administered to cells. Even though few or none of these techniques are presently considered so reliable it is routinely used clinically, the techniques can nonetheless be effective for gene delivery and expression if performed under the appropriate supervision of a physician. Thus, even gene therapy techniques which are not perfected to the degree of clinical routineness can be used to deliver a less active ALDH allele to cells.

In one embodiment, the less active ALDH allele is delivered to and expressed in liver cells. In some aspects, deliver to liver cells can be simpler than deliver to other cell types. The liver acts, in some regards, as a ‘filter’ for the blood stream, and can remove relatively large particles from the blood stream to a greater degree than other tissues. Because of this property, delivery to liver cells of an expression vector encoding a less active ALDH allele can be effected by administering relatively large particles to the blood stream. Among the large particles that can be delivered to liver cells are virus vectors and polymeric or other sustained release matrices which contain an expression vector. Of course, such large particles or individual expression vectors (e.g., plasmids, virus vectors, ‘naked’ linear DNA, etc.) can also be delivered to other cell types via the blood stream or, preferably, by local administration (i.e., not via the blood stream) to the tissue containing the cells. In one embodiment , a virus vector comprising a polynucleotide from which can be expressed an ASO which is completely complementary to a portion of an RNA molecule of the ALDH2 gene is administered locally to liver tissue (e.g., by direct hepatic injection), by delivery to liver tissue via the bloodstream, or both.

Virus vectors, such as those described in the Examples, are known to be useful for delivering genes to cells in an expressible form. Preferred virus vectors include retroviruses and adenoviruses.

Other Efficacious ASOs

The invention relates to the surprising discovery that an ASO which comprises a TCCC motif and is complementary to an RNA molecule, such as an mRNA or, preferably, a primary transcript, corresponding to a gene is efficacious for inhibiting expression of the gene. The ASO of the invention is complementary to a region of an RNA molecule corresponding to a gene, wherein the region of the RNA molecule comprises at least one GGGA motif. Also preferably, the ASO comprises not more than one nucleotide which is not complementary to the RNA molecule corresponding to the gene.

The ASO of the invention comprises between about twelve and about fifty nucleotides or even longer, up to the length of the complete gene, RNA transcript, or mRNA. Preferably, the ASO of the invention comprises between about fourteen and about thirty nucleotides; even more preferably, it comprises between about sixteen and about twenty-one or twenty-three nucleotides. The invention also features an ASO which comprises at least a pair of flanking nucleotides having a phosphorothioate or other modified (i.e., non-phosphodiester) linkage. The gene may, for example, be a gene of a DNA or RNA virus. Preferably the gene is an animal gene; even more preferably it is a human gene.

Oligonucleotides which contain phosphorothioate modification(s) are known to confer upon the oligonucleotide enhanced resistance to nucleases. As many as all of the nucleotide residues of an ASO may be phosphorothioate-modified, as may as few as one residue. Specific examples of modified oligonucleotides include those which contain phosphorothioate, phosphotriester, methyl phosphonate, short chain alkyl or cycloalkyl inter-sugar linkages, or short chain hetero-atomic or heterocyclic inter-sugar (“backbone”) linkages. In addition, oligonucleotides having morpholino backbone structures (U.S. Pat. No: 5,034,506) or polyamide backbone structures (Nielsen et al., 1991, Science 254: 1497) may also be used. Oligonucleotides which are methylated or alkylated at the 2′ hydroxyl position are also specifically included herein. These and other modified nucleotide residues, including peptide nucleic acids, for example, are known to those skilled in the art and are useful in the compositions and methods of the invention. Further by way of example, oligonucleotides comprising modified or non-naturally-occurring deoxyribonucleotide residues, modified or non-naturally-occurring ribonucleotide residues, or both, are likewise known and included in the compositions and methods of the invention.

The examples of oligonucleotide modifications described herein are not exhaustive and it is understood that the invention includes additional modifications of the ASOs of the invention which modifications serve to enhance the therapeutic properties of the ASO without appreciable alteration of the basic sequence of the ASO of the invention.

The antisense agents of the present invention may be incorporated in compositions suitable for a variety of modes of administration. One skilled in the art will appreciate that the optimal dose and methodology will vary depending upon the age, size, and condition of an animal. Optimal dose and route of administration are further dependent upon the bodily location of the organ, tissue, or cell to which the antisense agent of the invention is to be administered. Administration is generally continued until the cause or symptom of the disease or disorder is alleviated or cannot be detected.

Predicting the Efficacy of an ASO

The invention also includes a method for predicting whether an ASO will be efficacious for inhibiting expression of a gene, the method comprising determining whether the ASO is complementary to a portion of an RNA molecule corresponding to the gene, wherein that portion comprises a GGGA motif.

Methods of Making the Efficacious ASO of the Invention

The invention further includes a method for making an ASO which is efficacious for inhibiting expression of a gene having a corresponding RNA molecule. Such an ASO is made by synthesizing an oligonucleotide which comprises a TCCC motif and which is complementary to an RNA molecule corresponding to the gene. Methods for synthesizing an oligonucleotide having a selected nucleotide sequence are well known in the art. By way of example, a nucleotide sequence may be synthesized using an automated nucleotide synthesizing apparatus. The invention also includes, but is not limited to, ASOs made using this method.

The invention also includes an additional method of making an ASO which is efficacious for inhibiting expression of a gene having a corresponding RNA molecule which comprises an S¹GGGAS² sequence, wherein S¹ is a first RNA nucleotide sequence, and wherein S² is a second RNA nucleotide sequence. According to this method, the nucleotide sequence of the gene is obtained and a portion of the gene which encodes an S¹GGGAS² sequence in the corresponding RNA molecule is identified. The ASO is made by designing a nucleotide sequence which is complementary to the GGGA portion of the S¹ GGGAS² sequence and which is also complementary to at least a portion of one of the first RNA nucleotide sequence (i.e., S¹) and to at least a portion of the second RNA nucleotide sequence (i.e., S²). The invention also includes ASOs made using this method. This method can, for example, be used to make an ASO for inhibiting the expression of an ALDH2 gene in an animal.

Yet another method for making the ASO of the invention comprises making a plurality of ASOs, each of which comprises a TCCC motif and a randomly-generated sequence which flanks the TCCC motif on at least one side of the motif. Methods for synthesizing oligonucleotides comprising random sequences are well known in the art of molecular biology. The screening methods described herein may be used to screen the plurality of oligonucleotides to identify ASOs which are efficacious for inhibiting expression of a gene.

The ASOs of the invention include, but are not limited to, phosphorothioate oligonucleotides and other modifications of oligonucleotides. Methods for synthesizing oligonucleotides, phosphorothioate oligonucleotides, end-group-modified oligonucleotides, and otherwise modified oligonucleotides are known in the art (e.g. U.S. Pat. No: 5,034,506; Nielsen et al., 1991, Science 254: 1497).

Methods of Separating the Efficacious ASO from a Mixture of Oligonucleotides

The invention also includes methods of separating an ASO from a mixture of oligonucleotides, wherein the ASO is efficacious for inhibiting expression of a gene having a corresponding RNA molecule. These methods comprise contacting the mixture of oligonucleotides with a support which comprises a polynucleotide linked thereto, the polynucleotide comprising a portion having the sequence GGGA. After contacting the mixture with the support, the support is separated from the mixture, whereby efficacious ASOs remain bound to the support and are separated from the mixture. Various supports known in the art may be linked to the GGGA nucleotide sequence using known methods. By way of example, the sequence may be linked to cross-linked agarose beads or to a solid silica support. The polynucleotide may, for example, be all or a portion of the corresponding RNA molecule or a single strand of DNA having a nucleotide sequence homologous with the sequence of the corresponding RNA molecule.

Better separation of the efficacious ASO may be effected by treating the medium with an agent which causes dissociation of the efficacious ASO from the support. Once again, such agents are well known in the art and depend upon the type of support employed in the method. By way of example, when the support comprises the oligonucleotide comprising a portion which has a GGGA sequence linked to a solid silica matrix, the agent may be heat applied to a solution contacting the support, whereby the efficacious ASO dissociates from the support when the solution reaches the melting temperature of the ASO-oligonucleotide-GGGA sequence complex. Likewise by way of example, when the support is cross-linked agarose beads, agents such as solvents and salts, which interfere with hydrogen bonding between the ASO and the oligonucleotide comprising a portion which has a GGGA sequence, may be used to cause dissociation of the efficacious ASO from the support.

Another method for improving the separation of the efficacious ASO from the mixture comprises performing the methods described herein, and subsequently contacting the oligonucleotide mixture comprising the efficacious ASO with a second medium which comprises a portion of the corresponding RNA molecule linked to a second support.

Methods of Treating Diseases and Disorders Which are Characterized by the Presence of an RNA Molecule

The invention features methods of treating diseases and disorders which are characterized by the presence in affected cells of an animal afflicted with the disease or disorder of an RNA molecule which corresponds to a gene. The RNA molecule may, for example, be one which is normally expressed in cells and expressed at an abnormal level in affected cells, or it may be one which is expressed only in affected cells, for example, one which is expressed only in affected cells by way of infection of the cell or abnormal gene expression in the cell. The molecule may be an mRNA molecule, for example, and is preferably a primary transcript. The methods comprise administering to the cells an antisense agent comprising an ASO of the invention which is efficacious for inhibiting expression of the gene.

The ASO may be administered to the animal to deliver a dose of between 1 nanogram/kilogram/day and 250 milligrams/kilogram/day. Preferably, the dose is between 5 milligrams/kilogram/day and 50 milligrams/kilogram/day. Antisense agents that are useful in the methods of the invention may be administered systemically in oral solid dosage forms, ophthalmic, suppository, aerosol, topical, intravenously-, intraperitoneally-, or subcutaneously-injectable, or other similar dosage forms. In addition to an ASO, such pharmaceutical compositions may contain pharmaceutically acceptable carriers and other ingredients known to enhance and facilitate drug administration. Other possible dosage forms, such as nanoparticles, liposomes, resealed erythrocytes, and immunologically based systems may also be used to administer the antisense agent according to the methods of the invention. Furthermore, antisense agents may be delivered using ‘naked DNA’ methods, wherein the oligonucleotides are not complexed with a carrier, or using viral vectors, such as adenoviral vectors or retroviral vectors.

Some examples of diseases and disorders which may be treated according to the methods of the invention are discussed herein. The invention should not be construed as being limited solely to these examples, as other diseases or disorders which are at present unknown, once known, may also be treatable using the methods of the invention.

Treatment of Inflammatory Diseases

The invention also features methods of treating inflammatory diseases which are associated with tumor necrosis factor alpha (TNF-alpha). TNF-alpha is a pro-inflammatory cytokine which exhibits pleiotropic effects on various cell types and tissues both in vivo and in vitro. Local expression of TNF-alpha is essential for cell homeostasis, but overexpression of TNF-alpha has been linked to numerous inflammatory conditions such as rheumatoid arthritis, systemic lupus erythmatosis, multiple sclerosis, leprosy, septic shock, and inflammatory bowel disease. Various studies have also established that TNF-alpha levels are greatly elevated in the plasma of humans afflicted with alcoholic hepatitis and cirrhosis, and that high TNF-alpha levels are correlated with mortality (McClain et al., 1986, Life Sci. 39:1474-1485; Felver et al., 1990 Alcohol. Clin. Exp. Res. 14:255-259; Bird et al., 1990, Ann. Int. Med. 112:917-920; Khoruts et al., 1991, Hepatol. 13:267-276; Sheron et al., 1991, Clin. Exp. Immunol. 84:449-453). Efforts to inhibit or control TNF-alpha over-expression are useful for the treatment of a number of conditions, including those discussed herein.

The invention includes a method of inhibiting expression of TNF-alpha. The method is useful for treating an animal afflicted with a TNF-alpha-associated disease or disorder. The method comprises administering a composition to an affected cell of an animal afflicted with a TNF-alpha-associated disease or disorder, which composition comprises an ASO which comprises a TCCC motif and which is complementary to an RNA molecule corresponding to a gene which encodes TNF-alpha. Given the homology that exists among animal TNF-alpha (and other) genes, a strategy similar to that described herein may be employed to design ASOs useful for inhibiting expression of TNF-alpha (and other) genes in a human.

The composition may also comprise an oligonucleotide delivery agent, such as a liposome, a plasmid, a nanoparticle projectile, a viral vector, or the like, for delivering the ASO to the interior of the affected cell. In view of the present disclosure, the skilled artisan is enabled to design ASOs specifically useful in humans using the nucleotide sequence of the human TNF-alpha gene.

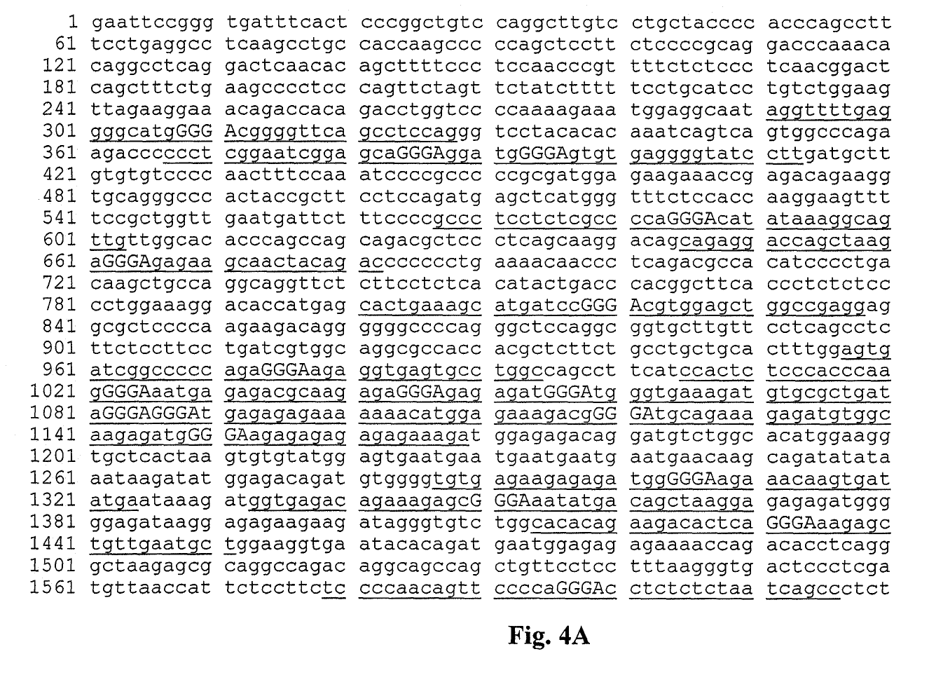

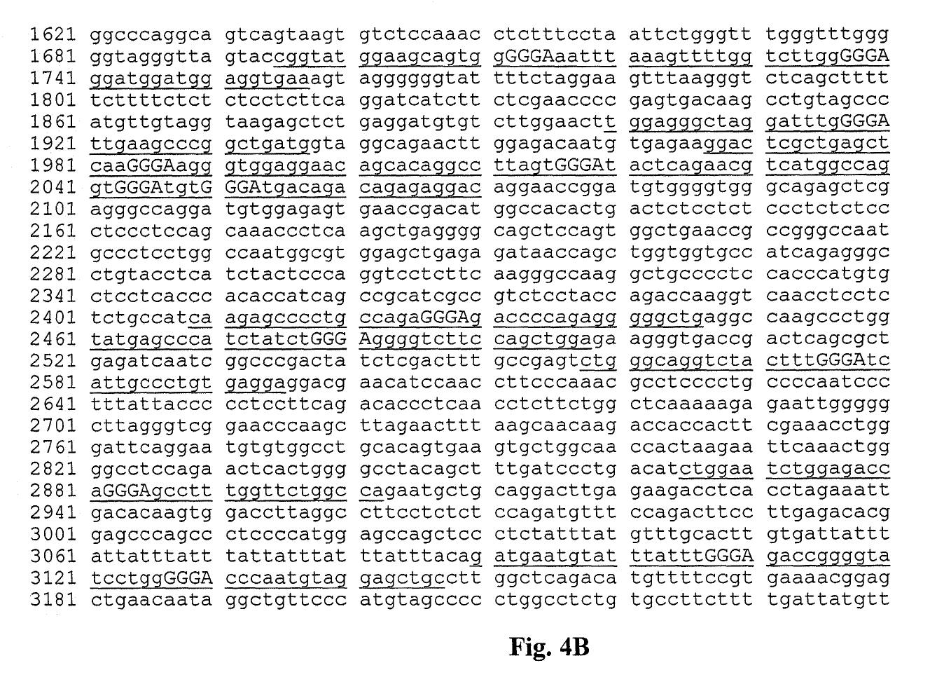

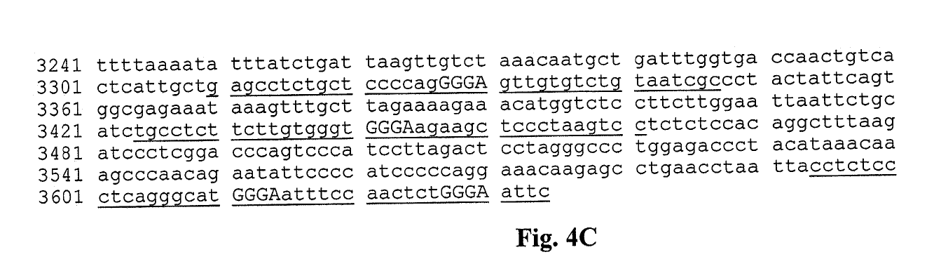

For example, the nucleotide sequence of the human TNF-alpha gene has been described (Nedwin et al., 1985, Nucl. Acids Res. 13:6361; GenBank Accession Nos. X02910 and X02159; FIG. 4). By identifying GGGA motifs in the nucleotide sequence of the primary transcript of the human TNF-alpha gene, one skilled in the art may design ASOs which are effective for inhibiting expression of the human TNF-alpha gene by selecting a region of the primary transcript corresponding to the human TNF-alpha gene, the region having a length of from 12 to 50 nucleotide residues, preferably having a length between 14 and 30 nucleotide residues, and more preferably having a length between 16 and 21 nucleotide residues. The region also comprises a GGGA motif. The efficacious ASO is designed by designing a nucleotide sequence which is at least 90%, preferably at least 95% complementary, and most preferably 100% complementary to the nucleotide sequence of the selected region of the primary transcript. The efficacious ASO may comprise modified or non-rally-occurring nucleotide residues, whereby the modified or non-naturally-occurring residues are capable of Watson-Crick-type base-pairing with the selected region of the primary transcript. Also preferably, the region of the primary transcript is selected such that it comprises a GGGA motif which is flanked by regions having high purine content or which are otherwise able to assume an A-form conformation.

By way of example, ASOs which are efficacious for inhibiting expression of human TNF-alpha and which have a length of up to 21 nucleotide residues may be made by synthesizing oligonucleotides which are at least 90%, preferably at least 95%, and most preferably 100% complementary to one of the underlined or double-underlined regions in FIG. 4 . Thus, an ASO which is efficacious for inhibiting expression of human TNF-alpha may have a nucleotide sequence which is complementary to up to, for example, sixteen to twenty-one consecutive nucleotide residues of the regions of the human TNF-alpha gene listed in Table 1, wherein the ASO is complementary to the GGGA motif in the region.

TABLE 1

| Region Designation | Nucleotide Residues^{A} |
|---|---|
| I | 291-328 |
| II | 367-413 |
| III | 567-603 |
| IV | 645-682 |
| V | 801-838 |
| VI | 957-994 |
| VII | 1005-1169 |
| VIII | 1287-1324 |
| IX | 1333-1370 |
| X | 1414-1451 |
| XI | 1579-1616 |
| XII | 1695-1757 |
| XIII | 1900-1937 |
| XIV | 1967-2070 |
| XV | 2409-2446 |
| XVI | 2461-2498 |
| XVII | 2558-2595 |
| XVIII | 2865-2902 |
| XIX | 3090-3147 |
| XX | 3310-3347 |
| XXI | 3424-3461 |
| XXII | 3594-3634 |

Note:
^{A}Nucleotide residues are identified using the numbering scheme used in FIG. 4. The indicated regions are inclusive of the nucleotide residues identified as boundaries.

ASOs which are complementary to an mRNA molecule corresponding to a gene and which are efficacious may be designed by an analogous method, wherein the nucleotide sequence of the mRNA molecule is substituted in place of the nucleotide sequence of the primary transcript in the preceding paragraph.

Methods of Inhibiting Gene Expression

The invention further features methods of inhibiting the expression of a gene in a cell, which methods comprise administering to the cell an ASO of the invention.

When gene expression is to be inhibited in the cell in vitro, ordinary transfection techniques are used to effect entry of the oligonucleotide into the cell. When gene expression is to be inhibited in the cell in vivo, then the above-described procedures are followed.

### EXAMPLES

The invention is now described with reference to the following examples. These examples are provided for the purpose of illustration only and the invention is not limited to these examples but rather encompasses all variations which are evident as a result of the teaching provided herein.

The materials and methods used in the experiments described in the Examples are now described.

Oligonucleotides

All phosphorothioate-modified oligonucleotides used in Examples 1-4 were synthesized and purified by Genset (La Jolla, Calif.). Prior to treatment of cells, oligonucleotides were sterilized by filtration through a filter having a pore diameter of 0.20 micrometer or through a filter having a pore diameter of 0.45 micrometer (Corning Glass Works, Corning, NY) and stored at −70° C. Oligonucleotide concentrations in solution were determined spectrophotometrically by measuring the ratio of absorbance at 260 nanometers to absorbance at 280 nanometers.

Cell Lines

WEHI 164 and H4-IIIC cell lines were purchased from American Type Culture Collection (ATCC, Rockville, Md.).

Rat Kupffer Cell Isolation

Kupffer cells were isolated from male rats (300-400 g body weight) by sequential digestion of the rat livers using pronase and type 1 collagenase followed by elutriation, as described (Bautista et al., 1992, Gen. Leukoc. Biol. 51:39-45). Purity of Kupffer cell preparations was assessed by staining the cells for peroxidase activity and assessing the ability of the cells to phagocytose 1 micrometer latex beads (Kamimura et al., 1995, Hepatol. 21:1304-1309). Purity of Kupffer cell preparations exceeded 85% in every experiment described herein. Viability of cells in Kupffer cell preparations was assessed using the Trypan blue exclusion test, and always exceeded 95%.

Approximately 10⁶ Kupffer cells were transferred to individual 35 millimeter diameter dishes, and Kupffer cells were further purified using the adherence method, as described (Kamimura et al., 1995, Hepatol. 21:1304-1309). Cells were typically incubated in RPMI medium with 10% fetal bovine serum for one day following the adherence method procedure, prior to the use of the cells in in vitro experiments.

Treatment of Cells with ASO

ASOs were suspended in LIPOFECTAMINE® (Life Technologies, Inc., Gaithersburg, Md.), a cationic liposome, prior to delivery to cultured rat Kupffer cells, as described (Tu et al., 1995, J. Biol. Chem. 270:28402-28407). Up to 12 micrograms of an ASO and 8 micrograms of liposomes were diluted separately in 100 microliters of OPTIMEM® (Life Technologies, Inc., Gaithersburg, MD) reduced serum medium. The two suspensions were gently mixed and the combined suspension was incubated at room temperature for 45 minutes to form oligonucleotide-liposome complexes.

Kupffer cells were rinsed twice with OPTIMEM® prior to the addition to the cell suspension of a mixture of 800 microliters of OPTIMEM® and 200 microliters of the combined suspension, which comprised oligonucleotide-liposome complexes. Cells were exposed to the complexes for 4 hours at 37° C., 5% (v/v) CO₂, and 100% humidity. Antibiotics were not present in the cell culture medium during liposome-mediated delivery.

Following treatment with the oligonucleotide-liposome complexes, the medium was removed, and the cells were washed twice with 37° C. OPTIMEM® and cultured in RPMI medium with 10% fetal bovine serum for an additional 17 hours. Tumor necrosis factor-alpha (TNF-alpha) expression was induced by addition to the cell culture medium of 10 nanograms per milliliter of lipopolysaccharide (LPS) for 2 hours. Following LPS treatment, the culture medium was removed and cells were stored at −70° C. until TNF-alpha assays were performed. Cells were rinsed twice with cold phosphate-buffered saline (PBS) and were lysed using a 5% (w/v) SDS solution prior to protein determination.

Extraction of Cellular RNA and Protein

Total cellular RNA was prepared from PBS-rinsed cells using TRI REAGENT® (Life Technologies, Inc., Gaithersburg, Md.) as described (Tu et al., 1995, J. Biol. Chem. 270:28402-28407). RNA concentration was determined by measuring the ratio of absorbance at 260 nanometers to absorbance at 280 nanometers using a Beckman DU-640 spectrophotometer.

Total cellular protein was prepared by lysing PBS-rinsed cells using a 5% (w/v) SDS solution at room temperature overnight. Protein concentrations were determined using a MicroBCA protein assay kit (Pierce, Rockford, Ill.) according to the manufacturer's instruction.

TNF-Alpha Assays

TNF-alpha in cell culture supernatants was assayed by bioassay and ELISA methods. The bioassay was performed as described (Kamimura et al., 1995, Hepatol. 21:1304-1309), using WEHI 164 cells as a assay reactant. ELISA was conducted by using a Cytoscreen KRC3012 ELISA kit (Biosource, Camarillo, Calif.) according to the manufacturer's specifications. Supernatants containing a high level of TNF-alpha were diluted prior to assay to ensure reliable assay results. All samples were assayed in triplicate.

Northern Hybridization

Total cellular RNA was isolated as described (Tu et al., 1995, J. Biol. Chem. 270:28402-28407). A Northern blot was prepared using 5 micrograms of total RNA per lane, and was probed using ³²P-labeled cDNA encoding murine TNF-alpha, as described (Kamimura et al., 1995, Hepatology 22:1304-1309). Densitometric analysis of TNF-alpha mRNA was standardized by comparison with 18S rRNA hybridization.

Other methods which were used but not described herein are well known and within the competence of one of ordinary skill in the art of antisense technology and molecular biology.

### Example 1

Eliciting the Low-Activity ALDH Asian Phenotype by Administering an Antisense Oligonucleotide

Results in Aversion to Ethanol in Rats

The results presented in this Example demonstrate that administration of an ASO directed against the ALDH2 gene to cells inhibits expression of the gene in the cells, both in vitro and in vivo. These results also demonstrate that administration of the ASO to a mammal inhibits the mammal's consumption of ethanol.

The materials and methods used in this Example are now described. Other methods used are described previously in this specification or are well known and within the competence of one of ordinary skill in the art of antisense technology and molecular biology.

Chemicals

Unless otherwise indicated, chemicals used were purchased from Sigma Chemical Company (St. Louis, Mo.), except for sucrose, sodium pyrophosphate, sodium phosphate, magnesium chloride, perchloric acid, hydrochloric acid, acetonitrile, and isooctane, which were purchased from Fisher Scientific (Pittsburgh, Pa.).

Antisense Oligonucleotides

Phosphorothioated oligonucleotides with specific base sequences used for in vitro studies were manufactured by Genset Corporation (La Jolla, Calif.). Purified ASO-9 (for in vivo work and for the last two in vitro studies described in this Example) was purchased from Hybridon (Milford, Mass.). For the in vivo study, HPLC purified ASO-9 was dissolved in phosphate buffered saline (PBS) at a concentration of 20 milligrams per milliliter. A large stock solution was prepared a day ahead of the initiation of the in vivo study and single dose aliquots were stored at -70degrees Celsius to avoid multiple freeze-thaw cycles.

Cell Line

The rat hepatoma cell line H4-II-E-C3 (H4) was purchased from American Type Culture Collection (ATCC™, Rockville, Md.).

ASO Delivery to H4 Cells

H4 cells were seeded 18-24 hours prior to the ASO delivery at a density of 2.5×10⁶ cells per 100 millimeter culture dish. Oligonucleotides were prepared using cationic liposomes, LIPOFECTAMINE PLUS® (Life Technologies, Inc, NY). The procedure used to deliver oligonucleotides for a 24 hour incubation was essentially the same as those recommended by the manufacturer. For each 100 millimeter culture dish to be treated, oligonucleotide and PLUS® reagent (27 microliters) were complexed with 40 microliters of LIPOFECTAMINE® (2 milligrams per milliliter) to form the LIPOFECTAMINE® PLUS®-oligonucleotide complex in serum-free Dulbecco's modified Eagle's medium containing 4.5 grams per liter of L-glutamine (DMEM). The final LIPOFECTAMINE(® PLUS®T-oligonucleotide complex (800 microliters) was added to 7.2 milliliters of DMEM at 37 degrees Celsius. Prior to addition of the prepared LIPOFECTAMINE® PLUS®-oligonucleotide complex, the medium with serum was removed from the cells and the final 8 milliliters of prepared LIPOFECTAMINE® PLUS®-oligonucleotide complex was added to the culture dish and incubated for 6 hours in the absence of serum.

Thirty minutes prior to the end of the 6 hour delivery, serum was added back to the cells for overnight culture. Because the oligonucleotide concentration was reduced by addition of serum, a second addition of LIPOFECTAMINE(® PLUS(® Plus- oligonucleotide complex (280 microliters) was prepared to maintain the desired concentration. The second addition was prepared using oligonucleotide, 9.45 microliters of PLUS(® reagent and 14 microliters of LIPOFECTAMINE®. The final LIPOFECTAMINE® Plus-oligonucleotide complex was added to the culture dish at 6 hours with a mixture of 20% horse (2.2 milliliters) and 5% fetal bovine (0.6 milliliters) serum and incubated for an additional 18 hours.

Intravenous Administration of ASO-9

Male Lewis rats (Harlan, Indianapolis, Ind.) weighing 200-300 grams each were used. Prior to femoral vein catheterization, all rats were acclimated for at least 3 days to their cages. All animals were maintained on a 12 hour light/dark cycle and had free access to laboratory rodent diet 5001 (PMI Feeds, Inc, St. Louis, Mo.) and tap water. Once animals were acclimated, surgery was performed to insert a femoral venous catheter comprising a piece of flexible TYGON™ plastic tubing (0.015 internal diameter ×0.03 outer diameter). Patency of catheters was maintained by using a lock solution containing heparin (444 units/milliliter), dextrose (22%), and streptokinase (16,667 units/milliliter), and a small piece of monofilament which was inserted at the exposed end of the cannula to retain the lock solution.

Intraperitoneal Administration of ASO-9

Male Lewis rats were surgically implanted with 2-milliliter osmotic pumps (ALZET MINIPUMP® 2 M1-1) containing a solution of 25 milligrams/milliliter ASO-9 in PBS or PBS alone (control) in the intraperitoneal cavity. A priming dose of 20 milligrams/kilogram was administered intraperitoneally following implantation of the puMps. The pumps delivered ASO-9 at 24 milligrams/kilogram/day. Three days after pump implantation, animals were deprived of water overnight while rat chow remained accessible. After the overnight fluid deprivation, animals were offered 6% (v/v) ethanol as the only fluid and consumption was measured at hourly intervals for 5 hours. Rehydration is a prime physiological drive and animals will initially consume the fluid offered. After the initial bout, rejection to continue consuming the freely available fluid indicates an aversion to the fluid offered. The general protocol follows that described by Garver et al. (2000, Alcohol Alcohol. 35:435-438).

Mitochondrial Isolations

Upon completion of incubations, H4 cells were collected by removing the culture medium containing the LIPOFECTAMINE® PLUS®-oligonucleotide complex and washing (1×5 milliliters) with ice-cold mitochondrial isolation buffer (0.25 millimolar sucrose in 10 millimolar Tris-HCl, pH 7.4). Cells were collected and transferred to an ice-cold conical tube for centrifugation at 152×g for 5 minutes at 4° C. to concentrate the cells into a pellet which was then resuspended in 200 microliters of the isolation buffer. The concentrated cellular suspension was then homogenized and fractionated as previously described (Tank et al., 1981, Biochem. Pharmacol. 30:3265-75). The final purified mitochondrial pellet was resuspended in 250 microliters of 50 millimolar sodium pyrophosphate (pH 9.0), immediately frozen in liquid nitrogen, and stored at -70° C. until the time of analysis. Prior to analysis, the mitochondria were thawed on ice, solubilized by adding 1% Trition X-100, and incubated for 15 minutes on ice. In some studies (Study III, Table 5 and Table 7), the Triton X-treated mitochondria were transferred to a ultracentrifuge tube and centrifuged at 4° C. for 30 minutes at 69,500×g to remove insoluble particulates. 101451 Mitochondria from rat livers collected from control- (PBS) and ASO-9-treated animals were isolated as described (Tank et al., 1981, Biochem. Pharamcol. 30:3265-75).

The concentration of soluble mitochondrial protein isolated from H4 cells or rat liver was determined by the MicroBCA Protein Assay Reagent Kit (Pierce, Rockford, Ill.) as described by the manufacturer, using bovine serum albumin as the standard.

Acetaldehyde Determination by HPLC

Plasma acetaldehyde concentration was determined by HPLC as described (Garver et al., 2000, Alcohol Alcohol. 35:435-438). Determination of ALDH2 and GDH Activity

The activity of low-Kₘ ALDH in isolated mitochondria was assayed as described (Garver et al., 2000, Alcohol Alcohol. 35:435-438) with minor modifications. The assay was initiated by addition of propionaldehyde at a final concentration of 14 micromolar, and contained 80 micromolar NAD+and 2.5 millimolar magnesium chloride (Takahashi et al., 1980, J. Biol. Chem. 255:8206-8209). The reaction was linear with time, and ALDH activity was expressed as nanomoles of NADH per minute per milligram protein.

Glutamate dehydrogenase (GDH) activity was assayed as described (Schmidt, 1983, In “Methods of Enzymatic Analysis, Vol. III,” H.U. Bergmeyer editor, pp. 650-656) with minor modifications. The assay was performed at 37 degrees Celsius in a 400 microliter reaction mixture containing 25 micrograms of soluble mitochondrial protein. The reaction was linear with time, and GDH activity was expressed as the oxidation of NADH to NAD⁺ in micromoles of NAD per minute per milligram of protein.

Determination of ALDH2 Half-Life by Cycloheximide (CHX)

Studies were conducted to determine the optimal concentration of CHX required to inhibit (by >90%) protein synthesis in H4 cells. Cell were seeded at a density of 16×10⁶ cells per T75 flask 24 hours prior to treatment with CHX in order to ensure that they were in a stationary phase of growth. The incubation medium containing serum was removed from the cells, and the cells were washed twice with 5 milliliters of methionine-free DMEM (L-glutamine was added; Life Technologies, N.Y.). Cells were cultured for 1 hour in 8 milliliters of methionine-free DMEM containing 1, 3, 5, 7 or 10 micrograms/milliliter CHX, after which a 15-minute pulse of 35 S-labeled-methionine (1.5 microCurie, specific activity I Curie per micromole) was added to the flask. After the pulse, ³⁵S-methionine was removed from the flask and the cells were washed twice with 5 milliliters PBS. Cells were lysed in 10 milliliters of a solution containing 0.15 molar KCl and 1 millimolar EDTA (pH =8.0) and processed for radioactivity determination essentially as described (Reel et al., 1968, Proc. Natl. Acad. Sci. USA 61:200-206). The total cellular protein content of cells treated with or without CHX was determined as described above.

The half-life of ALDH2 (time to reduce the activity by 50%) in the H4 cells was determined in the presence of 5 micrograms/milliliter CHX. H4 cells (3 replicates/time per experiment) were treated for 1, 5, 7, 12, or 24 hours with CHX in DMEM without serum present. The mitochondrial aldehyde dehydrogenase activity remaining at the different times of incubation with CHX was determined. Sequencing of H4-II-E-C3 ALDH2-1 cDNA by Reverse Transcriptase-Polymerase Chain Reaction (RT-PCR)

The published cDNA sequence of the mitochondrial aldehyde dehydrogenase in the Sprague-Dawley rat (Farres et al., 1989, Eur. J. Biochem. 180:67-74) was used to design PCR primers to amplify the cDNA obtained from the mRNA isolated from the H4 cell line. Total cellular RNA was isolated using TRI-REAGENT® (Molecular Research Center, Inc. Cincinnati, Ohio), and the first strand cDNA was made using a First Strand cDNA Synthesis Kit using random hexamer primers (Amersham Pharmacia Biotech Inc., NJ) according to the manufacturer's protocols. Three sets of PCR primers were designed to amplify 3 fragments of 476, 753, and 598 base pairs which combined constituted 96.7% of the published Sprague-Dawkey rat sequence. All fragments were gel purified from 1% agarose gels using Qiagen's gel purification kit (Qiagen, Calif.) and the forward primer of each fragment was used in sequencing reactions.

Determination of mRNA Expression by Competitive RT-PCR

Competitive RT-PCR for ALDH was developed by first designing PCR primers to amplify a 584 base pair fragment from Sprague-Dawley rat cDNA. The primers used to generate the 584 base pair fragment (refered to as a parent fragment) were specific to mitochodrial ALDH. The parent fragment was amplified from the cDNA template under the following conditions: denaturation at 95° C. for 3 minutes, then 30 cycles of 95° C. for 1 minute, 63° C. for 30 seconds, 72° C. for 1.2 minutes, and after the last cycle, a final extension at 72° C. for 10 minutes. The resulting band was purified from a 1% agarose gel. An internal standard, which served as a competitor for primers of the parent fragment during reverse transcription (as recombinant RNA, rcRNA) and PCR (as first strand cDNA), was constructed using a technique similar to that described by Wang et al. (1989, Proc. Natl. Acad. Sci. USA 86:9717-9721). The same forward primer mentioned above and a new reverse primer, comprised of the previous reverse primer sequence and an added reverse primer sequence, was used to amplify a smaller internal standard fragment of 429 base pairs.

The PGEM-T EASY VECTOR® (Promega, Wis.) containing the 429 base pair recombinant DNA fragment (rcDNA) was transformed into DH5 alpha competent E. coli cells according to the manufacture's protocol (Life Technologies, NY). The plasmid DNA was linearized using Not I restriction enzyme and then purified before use as template in the in vitro transcription reaction. The in vitro transcription reaction for generating RNA to be used as competitor in the RT-PCR reaction was carried out using the RIBOPROBE® In Vitro Transcription System (Promega, Madison, Wis.). The in vitro transcription and RNA purification was performed as recommended in the Promega technical manual.

A streamlined procedure similar to that reported by Heuvel et al. (1993, Biotechniques 14:395-398) for design of a recombinant RNA (rcRNA) was used to make the GDH internal standard which eliminated any subcloning and relied only on PCR to obtain the rcDNA template for in vitro transcription. Primers were designed to amplify a 782 base-pair parent fragment. The cDNA template for GDH was obtained by reverse transcription of RNA isolated from H4 cells using random hexamers (pd(N)₆ from Amersham Pharmacia Biotech, NJ) at 20 nanograms/20 microliter RT reaction and Omniscript RT (Qiagen, Calif.). The GDH parent fragment primers were then used to amplify the 782 base pair fragment by PCR using similar conditions to those described above for ALDH using 55° C. for primer annealing. The PCR product was electrophoresed on a 1% agarose gel and the 782 base pair parent fragment was purified from the gel. The purified fragment was amplified with a newly designed forward primer and the same reverse primer as mentioned above. The smaller fragment was then used as the DNA template for in vitro transcription to make RNA which served as the GDH internal standard for competitive RT-PCR reactions with sample RNA.

Sample analysis for ALDH or GDH mRNA expression was performed using 2 micrograms of total RNA spiked with a range of internal standard RNA (0-100 picograms ALDH RNA and 0-32 nanograms GDH RNA) depending on the anticipated gene expression. OMNISCRIP™ RT was used for all the reverse transcription reactions according to the Qiagen protocol using random hexamers (Amersham Pharmacia Biotech, NJ) to prime first strand cDNA synthesis. The parent fragment and internal standard bands were analyzed on a Kodak Digital Science Image Station 440CF (Eastman Kodak Co, NY) by analyzing the net intensity of the UV flourescence emitted by ethidium bromide in the bands. The actual amount of internal standard that was needed to compete for amplification with the parent fragment was determined by plotting the amount of internal standard against its corresponding net intensity ratio (internal standard band to parent fragment band). Using a linear relationship, the exact amount of internal standard needed to equally compete with the amount of sample mRNA present was determined by linear regression.

The results of experiments using ASO-9, a phosphorothioate-modified deoxyoligonucleotide containing a 5′-TCCC-3′ motif and directed against a portion of an ALDH2 mRNA molecule, are now presented. These experiments demonstrate that ASO-9 was effective for (i) reducing ALDH2 mRNA levels and mitochondrial ALDH2 activity in rat hepatoma cells in vitro, (ii) reducing liver ALDH2 MRNA and mitochondrial ALDH2 activity in rat liver cells in vivo, (iii) increasing four-fold the plasma acetaldehyde levels following an oral dose of ethanol, and (iv) eliciting a marked reduction in ethanol consumption. The studies presented in this example demonstrate that an antisense oligonucleotide that anneals to a GGGA-containing portion of an RNA molecule of an animal's ALDH2 gene can elicit a marked reduction in voluntary ethanol consumption in the animal.

Low-Kₘ Mitochondrial Aldehyde Dehydrogenase (ALDH2) in Rat Hepatoma Cells and the Liver of the Lewis Rat

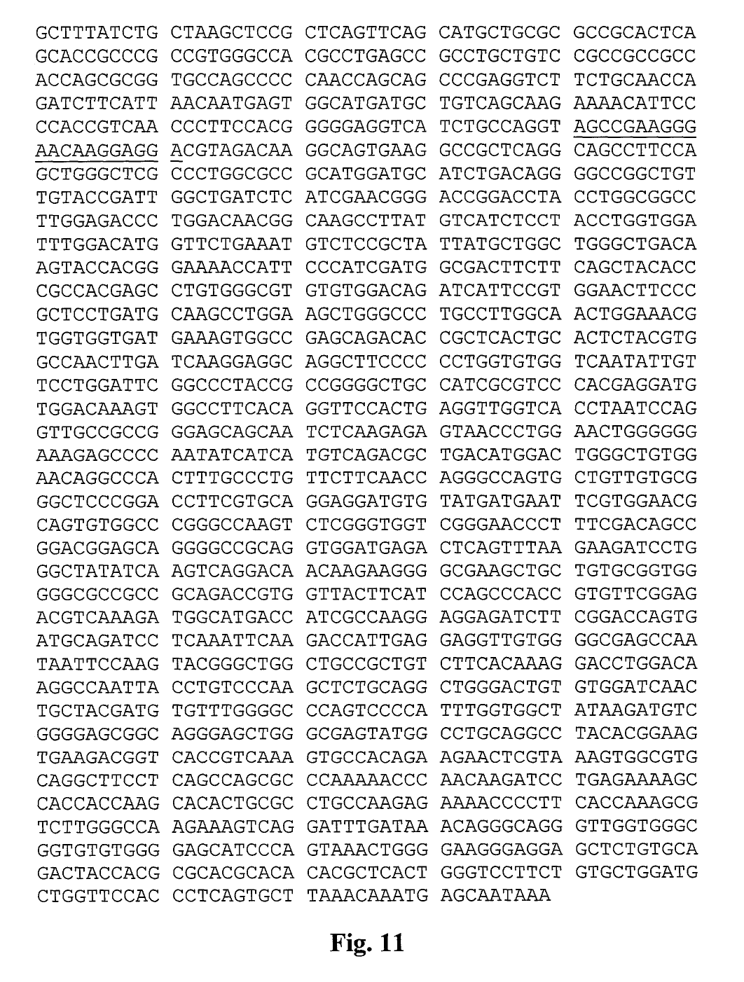

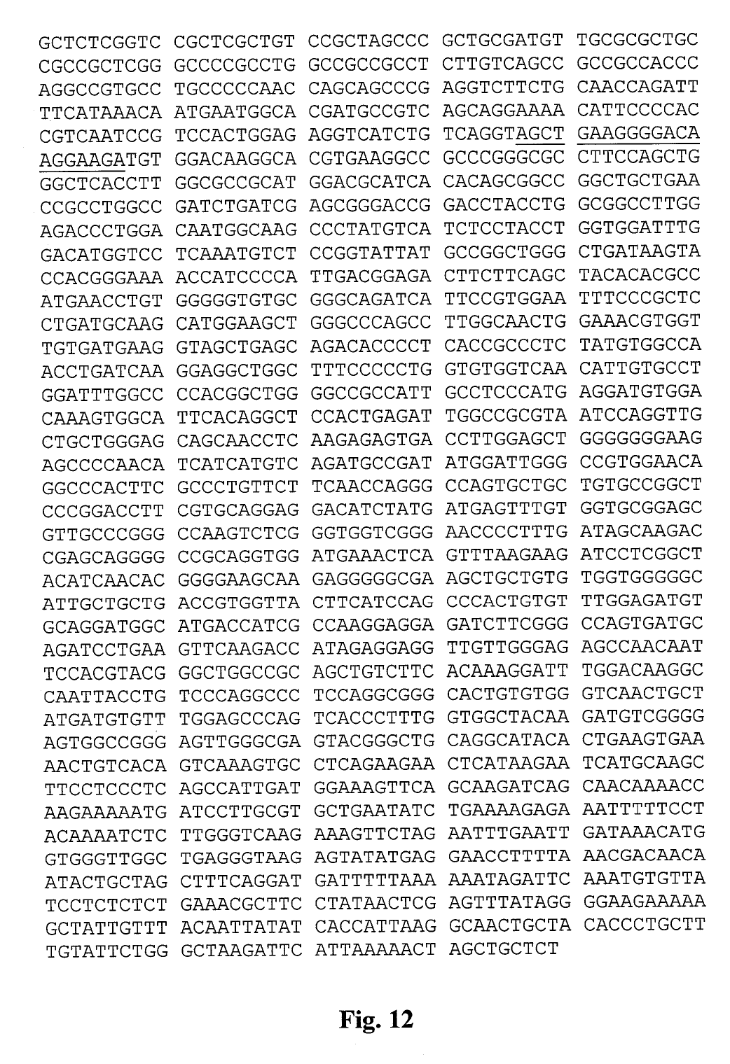

Lindahl and associates (Huang et al., 1990, Arch. Biochem. Biophys. 277:296-300) have reported the existence of a mitochondrial ALDH2-1 in rat hepatoma H4-II-E-C3 cells (referred to as the H4 cell line), with characteristics similar to ALDH2 in rat and human liver mitochondria. The ALDH2-1 cDNA sequence has been reported for the Sprague-Dawley rat (Farres et al., 1989, Eur. J. Biochem. 180:67-74). Since the H4 cell line was derived from a tumor from an AXC-Buffalo rat cross, it was necessary to confirm that the ALDH2-1 cDNA sequence in the H4 cell line and in the inbred Lewis rat were similar to that reported for the Sprague Dawley rat (i.e., GENBANK® accession no. NM_{—}032416; SEQ ID NO: 108; FIG. 11). We confirmed by RT-PCR and subsequent sequencing that the cDNA sequences of this isozyme in H4 cells and in rat are >99% homologous to the cDNA sequence for which PCR primers were designed, which comprised 96.7% of ALDH cDNA. We also confirmed that ALDH2 activity is present in mitochondria of H4 cells and has a Kₘ for acetaldehyde lower than 1 micromolar. Thus, the mitochondrial ALDH activity measured in the H4 cells has a high affinity for acetaldehyde, similar to the affinity observed in rat and human liver mitochondria (Klyosov et al., 1996, Biochemistry 35:4445-56).

Reducing ALDH2-1 Activity of H4 Cells Incubated in the Presence of ASO-9

When using an antisense molecule to inhibit specific protein synthesis, the half-life of the pre-formed protein must be considered. In order to determine the half-life of the mature mitochondrial ALDH2, cycloheximide (CHX) was used to arrest the synthesis of all cellular proteins, which allowed us to measure the decay of the existing ALDH2 enzyme. We first determined the minimum concentration of CHX required for maximal inhibition of protein synthesis in H4 cells, as assessed by the the rate of incorporation of ³⁵S-metioninine into total protein. CHX at a concentration of 5 micrograms/milliliter maximized inhibition of ³⁵S-methionine incorporation. At this concentration of CHX, methionine-derived 35S incorporation was inhibited by 85% over 24 hours. The residual incorporation could be due to post-translational incorporation of ³⁵S-sulfate, and the residual incorporation was not further reduced by increasing the concentrations of CHX. Cell viability under these conditions exceeded 90%. Under these conditions, ALDH2 activity in the H4 cells was reduced by 56% from control values in 24 hours. The calculated half-life of the mature ALDH2 in the mitochondria was 21.6 hours. This half-life is in line with the half-life of 22 hours reported for Hela cells transduced with human ALDH2-1 (Crabb et al., 1998, Alcohol: Clin. Exp. Res. 22:780-781).

Presented herein are the results of antisense phosphorothioate deoxyoligonucleotide #9 (ASO-9), the most effective antisense molecule containing the TCCC motif that was studied.

Data in Table 5 show that following a 24-hour incubation of H4 cells with ASO-9, mitochondrial ALDH2 activity was reduced by 54.9±10.7% (mean±SEM). Incubation with LIPOFECTAMINE™ alone or LIPOFECTAMINE™ with control (i.e., non-ASO) oligonucleotide(s) resulted in no reduction in ALDH2 activity. Given a half-life of 21.6 hours, it was determined that de novo synthesis of ALDH2-1 was inhibited by >95% (i.e., 98.1% ±19%) following 24 hours of exposure to ASO-9. Thus, ASO-9 proved to be very effective in inhibiting synthesis of ALDH2. However, the exact mechanism by which this molecule elicited its effects was unknown. Therefore, studies were conducted to determine whether ASO-9 reduced ALDH2-1 mRNA levels.

TABLE 5
Effect of ASO-9 on Activity of the Low Kₘ Aldehyde Dehydrogenase (ALDH2) in
Mitochondria of H4-II-EC-3 Rat Hepatoma Cells.
Aldehyde Dehydrogenase Activity (nanomoles per minute per milligram of protein)

| | | Lipofectamine | Lipofectamine + | Lipofectamine + | | |
|---|---|---|---|---|---|---|
| | Control | (no oligo) | Control Oligo§ | ASO-9** | Inhibition | p-value |
| STUDY I* | 18.4 ± 0.8(4) | 16.4 ± 3.4(4) | 15.9 ± 3.4(4) | 3.9 ± 1.4(4) | 76.2% | <0.02 |
| STUDY II* | | 14.0 ± 2.2(4) | | 7.5 ± 1.3(4) | 46.5% | <0.02 |
| STUDY III‡ | | 37.1 ± 3.4(4) | | 21.5 ± 4.0(4) | 42.1% | <0.02 |

Where applicable, all oligonucleotides were maintained at a concentration of 1 micromolar throughout the 24-hour incubation. Values are reported as mean ± standard error of the mean where applicable.
*Studies I and II were conducted with mitochondrial fractions re-suspended in 1% Triton-X100.
‡Study III was conducted with mitochondrial fractions re-suspended in 1% Triton-X100 and centrifuged to remove non-soluble debris.
§Sequence 5′-CAGATGACCTCCCCCGTGGAA-3′ (SEQ ID NO: 97)
**Sequence 5′-TCCTCCTTGTTCCCTTCGGCT-3′ (SEQ ID NO: 98)

Reducing ALDH2 mRNA Levels in H4 cells Using ASO-9

The steady-state level of ALDH2 mRNA was determined by quantitative competitive RT-PCR. Table 6 shows that following 24 hour treatment with ASO-9, ALDH2 MRNA in the H4 cells was reduced by 84.8±4.7%. This result indicates that the mechanism of action for ASO-9 is mediated by RNA hydrolysis (e.g., that mediated by RNase H).

Specificity

The effect of minor sequence changes on the ASO-9 molecule was tested by incubating cells with phosphorothiate oligonucleotides containing 2-, 3-or 4-base mismatches compared to ASO-9. One of the mismatches was always located in the “TCCC” motif. The studies showed that mismatches of 2-, 3-or 4-bases in ASO-9 decreased the oligonucleotide's ability to reduce the levels of mRNA. The greater the number of mismatches incorporated into the sequence of ASO-9, the less effective the modified ASO-9 became (see Table 6; experiments 4 and 6).

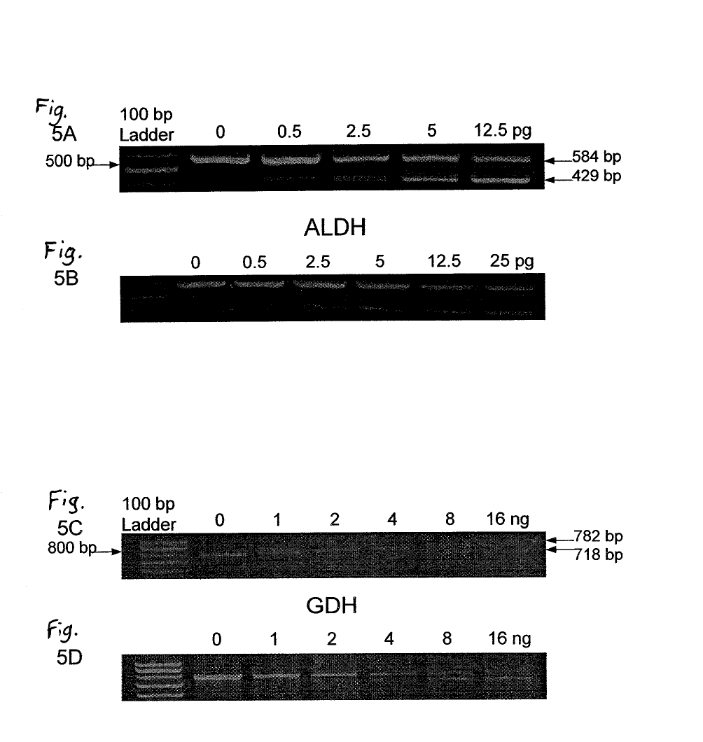

The specificity of ASO-9 was also demonstrated by determining mRNA levels of ALDH2 and of glutamate dehydrogenase (GDH), another mitochondrial enzyme, in the same cells. FIG. 5 shows the reduction in mRNA ALDH2 afforded by ASO-9 (see FIG. 5A) and its ineffectiveness on GDH mRNA levels (FIG. 5B). H4-II-E-C3 hepatoma cells were incubated with LIPOFECTAMINE PLUS® in the presence of 1 micromolar ASO-9 (FIGS. 5A and 5C) or the 4-base mismatch of ASO-9 (FIGS. 5B and 5D). The concentration shown represents the amount of ALDH2 MRNA competitor in picograms added per microgram total RNA (FIGS. 5A and 5B) and GDH MRNA competitor in nanograms added per microgram total RNA (FIGS. 5C and 5D). As can be observed, less competitor was necessary to compete with ALDH2 mRNA in ASO-9 treated cells than in cells treated with the 4-base mismatch of ASO-9 (control). In three replicates, the relative concentration of ALDH mRNA for 4-base mismatch control cells was 21.8±18.3 picograms/milligram total RNA and that for the ASO-9 treated cells was 5.6±0.5 picograms/milligram total RNA.

ASO-9 did not affect the GDH mRNA levels when compared to the 4-base mismatch oligonucleotide. In three replicates, the relative concentration of GDH mRNA for 4-base mismatch control cells was 9.65±0.19 nanograms/milligram total RNA and that for the ASO-9 treated cells was 9.26±0.18 nanograms/milligram total RNA.

TABLE 6

| | |
|---|---|
| ALDH2 mRNA | |
| (picogram mRNA per | |
| microgram total RNA | Inhibition |

EXPERIMENT #1

| | | |
|---|---|---|
| Control Oligonucleotide (1.0 micromolar) | 12.5 | NA |
| ASO-9 (1.0 micromolar) | 2.5 | 80% |
| ASO-9 (0.5 micromolar) | 2.0 | 84% |
| ASO-9 (0.25 micromolar) | 1.5 | 88% |

EXPERIMENT #2

| | | |
|---|---|---|
| Control Oligonucleotide (1.0 micromolar) | 100 | NA |
| ASO-9 (1.0 micromolar) | 3 | 97% |

EXPERIMENT #3

| | | |
|---|---|---|
| Control Oligonucleotide (0.5 micromolar) | 25 | NA |
| ASO-9 (0.5 micromolar) | 5 | 80% |

EXPERIMENT #4

| | | |
|---|---|---|
| 2 bp mismatch of ASO-9 (1.0 micromolar) | 7.5 | NA |
| ASO-9 (1.0 micromolar) | 2.5 | 67% |

EXPERIMENT #5

| | | |
|---|---|---|
| LIPOFECTAMINE Plus (no ASO) | 23.4 | NA |
| ASO-9 (1.0 micromolar) | 4.7 | 80% |

EXPERIMENT #6

| | | |
|---|---|---|
| 3 bp mismatch of ASO-9 (1.0 micromolar) | 12.95 | NA |
| 4 bp mismatch of ASO-9 (1.0 micromolar) | 21.8±8.3 | NA |
| ASO-9 (1.0 micromolar) | 5.6±0.5 | 74%* |

Control oligonucleotide in experiments 1-3:

| | |
|---|---|
| 5′-CGTCTTCACTTCCGTGTAGGC-3′ | (SEQ ID NO: 99) |

2 base mismatch of ASO-9 in experiment #4:

| | |
|---|---|
| 5′-TCCTCG TTGTTCGCTTCG GCT-3′ | (SEQ ID NO: 100) |

3 base mismatch of ASO-9 in experiment #5:

| | |
|---|---|
| 5′-TCCTCG TTGTTCGCATCG GCT-3′ | (SEQ ID NO: 101) |

4 base mismatch of ASO-9 in experiment #6:

| | |
|---|---|
| 5′-TCCACG TTGTACGCATCG GCT-3′ | (SEQ ID NO: 102) |

*Inhibition versus the 4-base pair mismatch. Mean inhibition by ASO-9: 84.8±4.7%

C. In vivo inhibition of ALDH2 activity and mRNA by antisense oligonucleotide ASO-9

Because ASO-9 proved to be most effective in reducing ALDH2 activity in cell culture and was both sequence- and gene-specific for ALDH2, we determined whether this molecule could reduce the activity of hepatic ALDH2 in vivo. GDH activity was determined as a control mitochondrial enzyme. ALDH2 and GDH gene expression in vivo was determined by analysis of mRNA. Antisense ASO-9 in PBS (15 milligrams/kilogram) or PBS alone was administered for 4 days via an indwelling femoral vein catheter. Twenty-four hours after the last dose of ASO-9 or PBS, animals received an oral dose of 1 gram per kilogram body weight ethanol (from a solution of 7% (v/v) in water) and were sacrificed 60 minutes later for determination of mRNA levels, enzymatic activities, and plasma acetaldehyde levels. Administration of ASO-9 to the rats resulted in a 50% reduction in ALDH2 mRNA, compared to rats to which PBS (control) was administered. The results of these experiments are listed in Table 7. Administration of ASO-9 reduced ALDH2 activity by 38-45% and resulted in a four-fold increase in plasma acetaldehyde levels following ethanol administration, compared to acetaldehyde levels in control animals that received the same dose of ethanol. The results of these experiments are listed in Table 8. Glutamate dehydrogenase activity and GDH mRNA levels were not affected by ASO-9, demonstrating the specificity of ASO-9 for modulating ALDH2 gene expression in vivo.

TABLE 7

| | ALDH2-1# | GDH# | Inhibition* |
|---|---|---|---|
| PBS | | | |
| 1 | 13.0 | 2.6 | NA |
| 2 | 15.3 | 2.6 | NA |
| 3 | 16.1 | 2.3 | NA |
| 4 | 19.6 | — | NA |
| | 16 ± 1.4 | 2.5 ± 0.1 | |
| ASO-9 | | | |
| 1 | 7.4 | 2.3 | 53.8 |
| 2 | 9.6 | 2.7 | 40.0 |
| 3 | 6.4 | 2.6 | 60.0 |
| 4 | 8.4 | — | 47.5 |
| | 8.0 ± 0.7 | 2.5 ± 0.1 | 50.3 ± 4.2% |

#ALDH2-1 quantities given as picograms of mRNA per microgram of total RNA. Quantities for GDH are given as nanograms of mRNA per microgram of total RNA
*Inhibition for each animal in ASO-9 group calculated from mean ALDH2-1 mRNA expression of PBS group
t-test of PBS versus ASO-9, p < 0.007

TABLE 8

| | ALDH | GDH | | Plasma |
|---|---|---|---|---|
| Treatment | Specific | Specific | ALDH/GDH | Acetaldehyde |
| Group | Activity# | Activity# | (Ratio) | (micromolar) |
| PBS | | | | |
| 1 | 58.9 | 6.4 | 9.2 | 1.5 |
| 2 | 48.2 | 6.4 | 7.5 | 1.9 |
| 3 | 42.9 | 7.1 | 6.0 | 1.7 |
| 4 | 58.9 | 8.4 | 7.0 | 3.6 |
| | 52.2 ± 4.0* | 7.1 ± 0.5* | 7.4 ± 0.7* | 2.2 ± 1.0* |
| ASO-9 | | | | |
| 1 | 37.5 | 7.7 | 4.9 | 7.5 |
| 2 | 37.5 | 7.7 | 4.9 | 6.1 |
| 3 | 32.2 | 8.4 | 3.8 | 7.5 |
| 4 | 42.9 | 8.4 | 5.1 | 8.1 |
| 5 | 37.5 | 7.7 | 4.9 | 9.4 |
| 6 | 32.2 | 7.7 | 4.2 | 11.4 |
| | 36.6 ± 1.6** | 7.9 ± 0.2‡ | 4.6 ± 0.2§ | 8.3 ± 1.9*** |

#Specific Activity values are given as nanomoles/minute/milligram protein.
*Group values are means ± sem.
**Effect of ASO-9 on mitochondrial ALDH activity: p < 0.01
‡Effect of ASO-9 on mitochondrial GDH activity: NS
§Effect of ASO-9 on ALDH/GDH ratio: p < 0.015
***Effect of ASO-9 on plasma acetaldehyde levels: p < 0.002

Inhibition of Ethanol Consumption Following Treatment with ASO-9

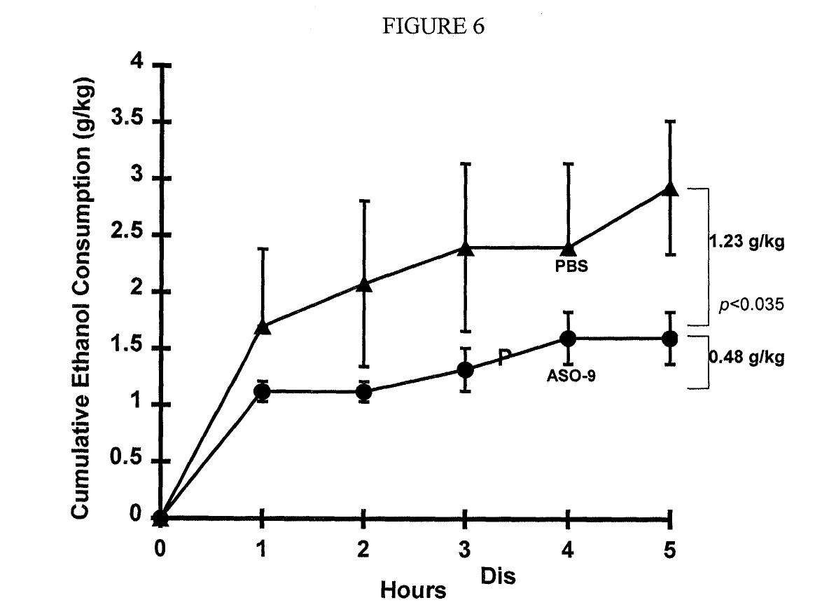

Studies were performed to determine if administration of ASO-9 could establish an aversion to ethanol and reduce ethanol consumption. Rats were surgically implanted with osmotic pumps which delivered ASO-9 at 24 milligrams/kilogram/day intraperitoneally. Three days after pump implantation, animals were deprived of water overnight. Thereafter, animals were offered 6% (v/v) ethanol as the only fluid, and consumption of the ethanol solution was measured at hourly intervals for 5 hours. A reduction in ethanol consumption after the first bout indicated an aversion to the fluid offered. Cumulative ethanol consumption (mean±SEM) at each hourly interval is shown in FIG. 6.

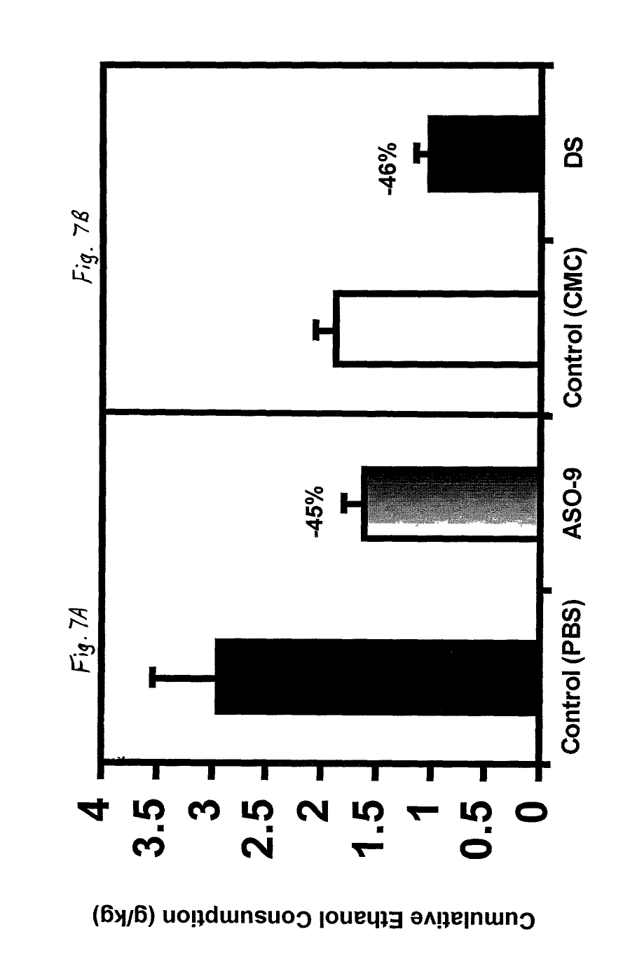

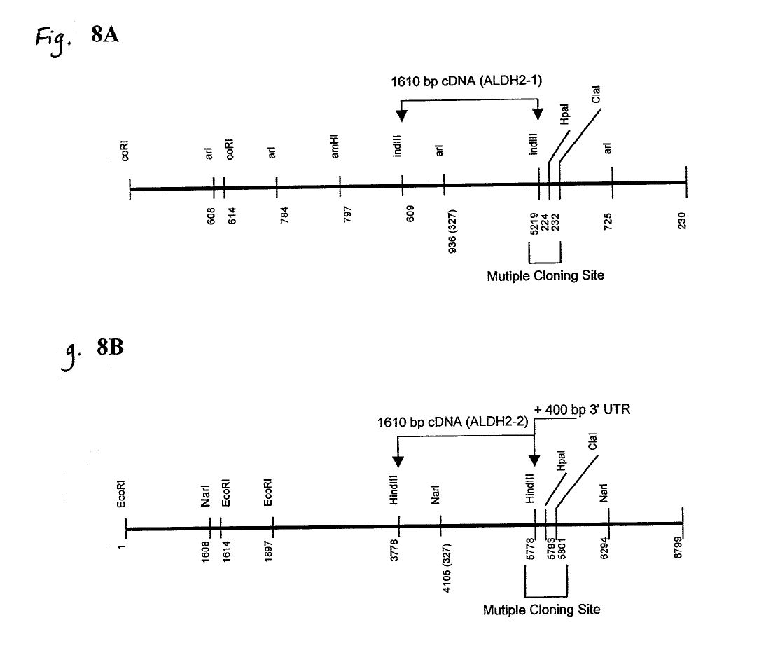

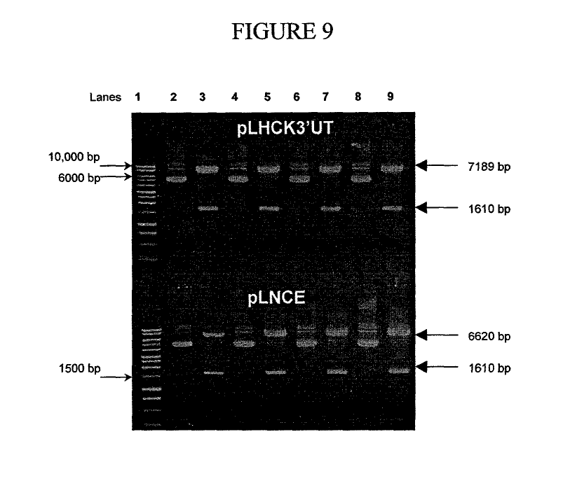

Initial ethanol consumption was similar both the ASO-9 and control (PBS) groups during the first hour of ethanol presentation, amounting to 1.12±0.09 grams ethanol/kilogram (rats treated with ASO-9) and 1.70±0.68 grams ethanol/kilogram (control rats). After the first hour of ethanol presentation, consumption in the 1-5 hour interval was reduced significantly (p<0.0 15) in the ASO-9 group (0.48±0.23 grams ethanol/kilogram) relative to the control group (1.22±0.16 grams ethanol/kilogram; FIG. 6). This represents a 61% reduction in ethanol consumption in ASO-9 treated animals. As indicated in FIG. 7A, cumulative ethanol consumption at 5 hours (which includes the amount consumed in the first hour of presentation) was 1.599±0.23 grams ethanol/kilogram (ASO-9-treated rats) and 2.928±0.59 grams ethanol/kilogram (control rats), corresponding to a 45% reduction (p<0.035) in total ethanol consumption in the ASO-9-treated animals relative to controls. This reduction is similar to the 46% reduction in ethanol consumption observed in studies in which rats were treated with disulfiram (a non-specific drug) in order to induce aversion to ethanol (FIG. 7B; data from Garver et al., 2000, Alcohol Alcohol 35:435-438).

The Experiments presented in this Example demonstrate that administration of an ASO that anneals with a GGGA-containing region of the ALDH2 gene of an animal can inhibit ALDH2 activity in the animal and induce aversion of the animal to ethanol.

### Example 2

Transduction of Rat Hepatoma H4 Cells with Human ALDH2-l or ALDH2-2 cDNA

The results presented in this Example demonstrate that a relatively inactive allele of the ALDH2 gene can be expressed in mammalian cells, and that expression of that allele inhibits ALDH activity in the cells. These results indicate that expressing this allele in mammalian liver cells can decrease acetaldehyde metabolism, thereby increasing the unpleasant effects of ethanol consumption and inducing ethanol aversion.

The materials and methods used in this Example are now described. Other methods used are described previously in this specification or are well known and within the competence of one of ordinary skill in the art of antisense technology and molecular biology.

Virus Vector Preparation

The retrovirus vectors were stored on 3M filter paper, and the DNA was eluted from the filter paper with TE buffer (300 microliters). In order to obtain enough vector DNA to transfect the PA317 amphotropic retrovirus packaging cell line (see below), the vector DNA eluted from the filter paper was used to transform competent E. coli cells. The competent cells were thawed on wet ice and a 50 microliter aliquot of the cells was placed into a chilled 1.5 milliliter microcentrifuge tube. Vector DNA (5 microliters of filter paper eluant) was pipetted through the cells while being dispensed. The tube was gently mixed and the cells were placed back on ice for 30 minutes in order for the cells to take up the vector DNA. The cells were heat-shocked for 20 seconds at 37° C. and placed immediately on ice for 2 minutes. Room temperature LB medium (250 microliters) was added to the microcentrifuge tube and a hole was made in the cap with an 18 gauge sterile needle. The cap was closed, and the tube was placed in the shaking-incubator at 37° C. and 250 rpm for 2 hours for expression. At the end of 2 hours, 100 microliters of the cells were spread on LB agar plates containing 100 micrograms per milliliter ampicillin and 50 micrograms per milliliter Xgal and placed in the incubator at 37° C. overnight. White colonies containing vector DNA were selected after overnight incubation and placed into a conical tube containing 5 milliliters of LB medium with 100 micrograms per milliliter ampicillin for additional overnight incubation at 37° C. in the shaking-incubator at 250 rpm.

Expanded E. coli was then lysed for vector DNA collection according to Qiagen's protocol for their plasmid mini-prep kit or by the lithium chloride method as described in Current Protocols in Molecular Biology (Chanda, “Minipreps of plasmid DNA,” Ausubel et al., Eds., 1991, Wiley, New York, p. 1.6.5-1.6.6).

The isolated and purified vector DNA was subjected to restriction digests with Hind III to confirm the presence of either ALDH2-1 or ALDH2-2 human cDNA. The following is an example of the Hind III digest set-up in a 20 microliter total reaction volume:

13.8 microliter nuclease-free H₂O

2.0 microliter Hind III 10X restriction Buffer E

3.0 microliter DNA (0.6-1 microgram)

0.2 microliter Bovine Serum Albumin (BSA, 10 microgram/microliter)

1.0 microliter Hind III restriction enzyme (Promega, Cat. #R604 1)

Control Vector Preparation

The pLNCE vector containing the wild-type human cDNA (ALDH2-1) was digested with Hind III to liberate the 1610 base pair cDNA. The digest was electrophoresed on a 1% agarose gel containing ethidium-bromide and the 6620 base pair band was electroeluted at 120 volts for 30 minutes in 1×TAE using a SPECTRA-POR™ MOLECULARPORIS™ membrane tubing (molecular weight cut off ca. 3,500, cat. #132720, Los Angeles, Calif.). The DNA was purified from the 1×TAE by extracting with 1 volume of TE-saturated phenol:chloroform:isoamyl alcohol (25:24: 1, pH 4.5), vortexing for 1 minute and centrifugation at 12000×g for 2 minutes. The upper aqueous phase was transferred to a fresh tube and 1 volume of chloroform:isoamyl alcohol (24: 1) was added and vortexed for 1 minute followed by centrifugation at 12000×g for 2 minutes. Again, the upper aqueous phase was transferred to a fresh tube and 0.5% volume of 7.5 molar ammonium acetate and 2.5 volumes of 100% ethanol were mixed and placed at −70° C. for a minimum of 30 minutes. The Eppendorf tube was then centrifuged at 12000×g for 20 minutes, the supernatant was removed, and the DNA pellet was washed with 1 milliliter 70% ethanol. The DNA pellet was air dried in the SPEED-VAC™ vacuum centrifuge and reconstituted in DNase-free water. DNA concentrations were then determined by assessing absorbance at 260 and 280 nanometers.

The backbone of the pLNCE vector with the Hind III sticky ends was ligated using T4 DNA ligase (Promega, Madison, Wis.).

Preparation of Infectious Virus

Infectious virus was prepared using the PA317 amphotropic retrovirus packaging cell line (American Type Culture Collection #CRL 9078). These cells were grown in Dulbecco's Modified Eagle's Medium containing 4.5 grams per liter of L-glutamine (Mediatech CELLGRO®; Herndon, Va.), 10% fetal bovine serum (Life Technologies, Carlsbad, Calif.) in 5% CO₂ at 37 degrees Celsius.

The PA317 cell line was transfected with the purified vector DNA by using calcium phosphate to complex with the DNA. Two solutions were prepared to form the CaPO₃-DNA complex for transfection of PA317 cells. Solution #1 of HEPES buffered saline (HeBS, 2×) was prepared as follows: 2.4 grams NaCl, 0.111 gram KCl, 0.0564 gram Na₂HPO₄.7 H₂O, 0.3 gram dextrose purchased from Fisher (Pittsburgh, Pa.) and 1.5 grams HEPES acid (catalog #H3375; Sigma Chemical Company, St. Louis, Mo.) were dissolved in sterile water, brought to a pH 7.07 with 5-6 drops of 10 normal NaOH, and filter sterilized. Solution #2 of calcium chloride (2.5 molar) was prepared by dissolving 36.74 grams of CaCl.2H₂O in 100 milliliters of HEPES (10 millimolar, 2.603 grams per liter).

In a polypropylene tube containing 0.5 milliliters of solution #1, Vector DNA (10-20 micrograms) was added and mixed (Tube 1). In a second polypropylene tube, 0.5 milliliters of solution #2 was added (Tube 2), and then the contents of Tube 1 were slowly added to Tube 2 while the solution in Tube 2 was constantly bubbled using a 2 milliliter disposable pipette and mechanical pipettor.

This allowed the DNA to form a fine white precipitate and the mixture was incubated for 30 minutes at room temperature prior to addition to the PA317 cells. The final 1 milliliter CaPO₃-DNA complex was added to a 100 millimeter culture dish that had 1×10⁶ cells plated in 8 milliliters of culture medium 24 hours prior to this addition of CaPO₃-DNA complex. Cells were incubated overnight (18 hours) at standard culture conditions with the CaPO₃-DNA complex and then glycerol-shocked the next morning.

Medium was aspirated from CaPO₃-DNA complex-treated cells after 18 hours, and 4 milliliters of a 15% glycerol (in 1×HeBS) was placed into the 100 millimeter culture dish for 2 minutes at 37° C. The glycerol shock medium was removed and cells were washed with once with 1×HeBS followed by the addition of standard prepared culture medium (8 milliliters). The cells were incubated for 72 hours under standard culture conditions in order to achieve release of infectious viral particles into the culture medium.

Infectious virus was collected at 72 hours. The medium (8 milliliters) containing the infectious virus was removed from the cells and placed into a 15 milliliters conical tube (Corning, N.Y.), and cellular debris was removed from the medium by centrifugation at 1000×g. The viral supernatant was removed without disturbing the debris pellet and infectious virus supernatant could be used immediately or stored at −70° C. until needed.

Infection of H4 Cells

Prepared virus supernatant containing human ALDH2-1 or ALDH2-2 CDNA was used to infect the H4 cell line. The H4 cells were infected by placing 1 milliliter of the virus supernatant into 5 milliliters of prepared culture medium. In addition, hexadimethrene bromide (POLYBRENE™; catalog #H9268; Sigma Chemical Company, St Louis, Mo.) at a concentration of 4 micrograms per milliliter was added to the final 6 milliliter volume of culture medium to enhance viral infection. The cells were cultured at standard conditions overnight (18 hours), and the next morning the medium containing the virus supernatant was removed and replaced with prepared culture medium. The cells were then cultured for 48-72 hours to allow expression of the transgene. At the end of 48-72 hours, geneticin G418 (Sigma Chemical Company, St. Louis, Mo; added 1 microliter per milliliter of culture medium of a 580 milligrams per milliliter PBS stock) or hygromycin (Boehringer Mannheim Indianapolis, Ind.; added 4 microliters per milliliter culture medium of a 50 milligrams/ml PBS stock) was added to the ALDH2-1 or ALDH2-2 transduced H4 cells, respectively, in order to select cells that took up the virus vector.

Quantitation of Transgene Expression by Competitive RT-PCR

A streamlined procedure similar to that reported by Heuvel et al. (1993, Biotechniques 14:395-398) for design of a recombinant RNA (rcRNA) was used to make the human ALDH2 internal standard as described for rat ALDH2 competitive RT-PCR described herein. Primers were designed to amplify a 357 base pair parent fragment. Using the pLNCE vector DNA, the human ALDH2 parent fragment (357 base pair) was amplified by PCR using these primers. The PCR conditions were as follows: 94° C. for 3 minutes, 30-50 cycles of (i) 94° C. for 1 minute, (ii) 55° C. for 30 seconds, and (iii) 72° C. for 1 minute, followed by a final extension at 72° C. for 10 minutes. The PCR product was run on a 1% agarose gel and the 357 base pair parent fragment was purified from the gel. The purified fragment was then amplified. The new forward primer was constructed to incorportate the T7 promoter sequence to the previous forward primer sequence.

The smaller 307 base pair fragment obtained from the amplification using these primers was then used as the DNA template for in vitro transcription to make RNA which served as the human ALDH2 internal standard for competitive RT-PCR reactions with sample RNA. The final internal standard band (amplified with the parent fragment primers during competitive RT-PCR) was 280 base pairs in length, because the 27 base pair T7 sequence is not amplified.

The results of the experiments performed in this Example are now presented. The studies presented herein were designed to address whether delivery of the human ALDH2-1 and ALDH2-2 genes via a retrovirus for transduction of rat hepatoma cell line H4-II-E-C3 (H4 cells) could (i) lead to a functional combined species ALDH2 tetramer and (ii) whether incorporation of the human mutant monomer (ALDH2-2) in this combined species tetramer could reduce mitochondrial ALDH activity significantly (e.g., by >60%).

The nucleic acid sequences of rat and human ALDH2 are 80% identical, and the encoded proteins exhibit >95% amino acid homology. Furthermore, both human and rat ALDH2 enzymes exhibit Kₘ values <1 micromolar. These studies demonstrate that incorporation of a mutant human monomer into a functional rat ALDH2 tetramer structure leads to a >60% reduction in hepatocyte ALDH2 activity, relative to a rat functional ALDH2 tetramer that has not incorporated a mutant human monomer. These data indicate that delivery of an ALDH2-2 allele to hepatocytes in vivo can lead to a significant systemic accumulation of acetaldehyde. Accumulation of acetaldehyde can establish aversion to ethanol in the mammal that can be maintained for extended periods of time, for example a month or longer, through a single delivery of the ALDH2-2 allele. For example, it has been shown that an insulin transgene in an adenoviral vector for the treatment of diabetes was effective for twelve weeks (Thule et al., 2000, Gene Therapy 7:1744-1752).

Expression of a mutant human ALDH2 monomer in the liver of a rat can lead to acetaldehyde accumulation and aversion to alcohol similar to that seen in humans of certain Asian populations who are either hetero- or homozygous for ALDH2-2. A desirable goal of a gene therapy approach for the treatment of alcoholism is to incorporate a mutant human ALDH2 monomer into the functional human ALDH2 tetramer so that accumulation of acetaldehyde leads to dysphoria and ultimately to ethanol aversion. Garver et al. (2000, Alcohol Alcohol. 35:435-438) demonstrated that ethanol aversion can be established in rats using disulfiram, a drug that is used to establish ethanol aversion in humans, indicating that the rat is an appropriate pre-clinical model for human ethanol aversion therapy.

Confirmation of Vectors

Retroviral vectors containing human ALDH2-1 (wild-type) or ALDH2-2 (mutant) were stored on filter paper. In order to confirm the presence of the two different forms of cDNA and the integrity of the constructs a restriction digest was performed. Following a restriction digest with Hind III, vector DNA and liberated human cDNA insert sizes were confirmed on a 1% agarose gel containing ethidium bromide.

Preparation of Empty Vector (Control)

Empty vector (i.e., without a cDNA insert) was prepared to determine endogenous ALDH2 activity in cells transduced with an empty vector (i.e., without the presence of the human ALDH2-1 or ALDH2-2 cDNA). The pLNCE vector was modified so that ALDH2-1 cDNA could be liberated using Hind III to digest the vector DNA. Two bands, a 1610 base pair band representing the ALDH2-1 CDNA, and a 6620 base pair band representing the vector backbone, were separated on a 1% agarose gel and the 6620 base pair vector backbone DNA was purified from the gel for ligation by electroelution.

ALDH Activity in Transduced H4 Cells

ALDH2 activity in H4 cells transduced with pLNCE vector that did not contain the ALDH2-1 cDNA insert (i.e., empty vector) was used as the standard. H4 cells that were transduced with wild-type (PLNCE) or mutant (pLHCK3′UT) ALDH2 cDNA were compared to the standard with regard to endogenous ALDH2 expression and activity. The mean±standard error of the mean specific activity that was obtained for control clones (N=14) was 51.6±1.0 nanomoles/minute/milligram soluble mitochondrial protein.

In order to determine if a functional ALDH2 tetramer could be produced by combining human and rat monomers, H4 cells were transduced with the human ALDH2-1 cDNA. These transduced cells increased or maintained the ALDH2 activity that was observed in the control group. The mean±standard error of the mean specific activity that was obtained for the pLNCE-transduced clones (N=15) was 54.7±2.6 nanomoles/minute/milligram soluble mitochondrial protein. Although the mean activity for this group is not statistically significant when compared to H4 cells transduced with empty vector (p<0.14), ALDH activity was increased by as much as 52% in some ALDH2-1 transduced clones, relative to the control group.

H4 cells were transduced with a retroviral vector containing human cDNA ALDH2-2 to see if the incorporation of a mutant monomer into the tetrameric structure would decrease the enzyme activity. Cells transduced with the human ALDH2-2 cDNA exhibited decreased ALDH2 activity, relative to control cells. The mean±standard error of the mean specific activity that was obtained for the pLHCK3′UT-transformed clones (N=22) was 40.4±2.1 nanomoles/minute/milligram soluble mitochondrial protein. The ALDH2-2 transduced clones exhibited a significant reduction in ALDH2 activity when compared to either empty vector (control) or ALDH2-1 (PLNCE) transduced cells with p-values of <0.00004 (empty vector v. ALDH2-2) and <0.0002 (ALDH2-1 v. ALDH2-2). Although the propionaldehyde substrate was converted to propionoacetate by about 125 seconds by ALDH2 in control or ALDH2-1 transduced clones in the assay system used, ALDH2 in some ALDH2-2 transduced clones had not exhausted the added substrate until about 230 seconds.

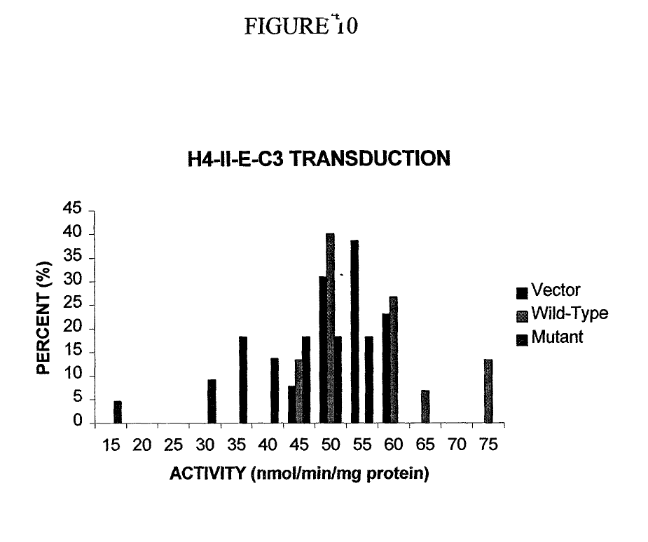

The bar graph in FIG. 10 shows the percentage of clones from each group (empty vector, ALDH2-1-transduced or ALDH2-2-transduced) that had ALDH2 specific activities of 15 to 75 nanomoles/minute/milligram of soluble mitochondrial protein. Table 9 summarizes the mean and range of ALDH2 specific activity for each tranduction group, as well as the p-values with respect to endogenous ALDH2 specific activity.

The range of ALDH2 specific activity observed overall in the empty vector and ALDH2-1 transduced clones was 45 to 60 nanomoles/minute/milligram with several ALDH2-1 transduced clones exhibiting ALDH2 activities as high as 75 nanomoles/minute/milligram of soluble mitochondrial protein. However, the ALDH2-2 transduced clones had a general range of ALDH2 specific activity of 30 to 55 nanomoles/minute/milligram of soluble mitochondrial protein, and two of the ALDH2-2 transduced clones had markedly lower specific activities of 15 nanomoles/minute/milligram of soluble mitochondrial protein.

TABLE 9

| Transduction | Number of | | |
|---|---|---|---|
| Type | Clones Tested | Specific Activity* | p-value*** |
| Empty Vector | 14 | 51.6 ± 1.0 (46-57)** | NA |
| ALDH2-1 | 15 | 54.7 ± 2.6 (42-78)** | <0.14 |
| ALDH2-2 | 22 | 40.4 ± 2.1 (15-54)** | <0.00004 |

*Values given in nanomoles/minute/milligram protein. Value represent mean ± SEM.
**Numbers in parentheses represent the range of specific activity.
***p-values represent comparison of transduction type with empty vector values

D. Glutamate Dehydrogenase Activity

Activity of the mitochondrial enzyme glutamate dehydrogenase (GDH) was assessed in the ALDH2-2-transduced H4 cells in order to determine if the activity of an unrelated mitochondrial enzyme was affected as a result of expression of the ALDH2-2 transgene. The GDH activity was measured in non-transduced H4 cells and in ALDH2-2 transduced H4 cell clones. Unlike ALDH2 activity, the GDH activity remained substantially the same in ALDH2-2-transduced clones (1,7 and 10), relative to non-transduced cells. The p-value in a t-Test comparing non-transduced H4 cells to the ALDH2-2-transduced clones was <0.8.

### Example 3

Screening ASOs Which are Efficacious for Inhibiting TNF-Alpha Expression

Based upon the primary transcript sequence of rat TNF-alpha (Shirai et al., 1989, Agric. Biol. Chem. 53:1733-1736), seventeen phosphorothioate-modified ASOs were designed which were complementary to different regions of the primary transcript, including the 5′-cap site, the translation initiation codon, various exon/intron junctions, the stop codon, and the 3′-untranslated region, as indicated in Table 2. The ability of each of these ASOs to inhibit expression of rat TNF-alpha in cultured rat Kupffer cells which were stimulated using bacterial lipopolysaccharide (LPS) was assessed by contacting aliquots of the cells with individual ASOs, culturing the aliquots for about seventeen hours, and then assessing TNF-alpha expression in the cells.

ASOs were delivered into cultured rat Kupffer cells using cationic liposomes (LIPOFECTAMINE®, Life Technologies, Inc., Gaithersburg, Md.). Cationic liposomes have been demonstrated to enhance cellular uptake and biological activity of phosphorothioate-modified oligonucleotides (Bennett et al., 1992, Mol. Pharmacol. 41:1023-1033; Bennett et al., 1993, J. Liposome Res. 3:85-102; Tu et al., 1995, J. Biol. Chem. 270:28402-28407). Although high concentrations of liposome has been reported to be toxic to some cell lines (Bennett et al., 1992, Mol. Pharmacol. 41:1023-1033), treatment of cultured rat Kupffer cells with 8 micrograms per milliliter LIPOFECTAMINE® for 4 hours did not inhibit rat TNF-alpha expression in Kupffer cells following LPS stimulation.

Table 2 summarizes results obtained using ASOs to inhibit rat TNF-alpha as described herein. Oligonucleotides having the sequence indicated in the table were synthesized and used to treat cultured rat Kupffer cells, as described herein. Expression of TNF-alpha protein was assessed following incubation of the cells and stimulation using LPS. TNF-alpha expression is reported in Table 2 as a mean percentage (±standard deviation) of TNF-alpha expression in control cells which were not treated with an ASO. Each ASO in Table 2 is reported using an identifier, a SEQ ID NO, a sequence listing, and an indication of the region of the primary transcript encoding rat TNF-alpha to which the ASO was designed to be complementary. “Putative tsp” denotes an ASO comprising a sequence complementary to the putative transcription start point. “AUG codon” denotes an ASO comprising a sequence complementary to the translational initiation site. “Ex. #/In. ##” denotes an ASO comprising a sequence complementary to the junction between exon # and intron ## of the RNA molecule encoding rat TNF-alpha. “3′-Untr. Reg.” denotes an ASO comprising a sequence complementary to a portion of the 3′-untranslated region of the RNA molecule encoding rat TNF-alpha. “Activation” denotes TNF-alpha expression which exceeded TNF-alpha expression which was observed in control cells.

TABLE 2

| | SEQ. ID | Oligonucleotide Sequence | Region of | TNF-alpha |
|---|---|---|---|---|
| Identifier | NO: | (5′ to 3′) | Primary Tran. | (% Con) |
| TJU-0641 | 1 | CCTCGCTGAGTTCTGCCGGCT | putative tsp | 97±8.9 |
| TJU-0796 | 2 | CCGTGCTCATGGTGTCCTTTC | AUG codon | 52±5.7 |
| TJU-0807 | 3 | GATCATGCTTTCCGTGCTCAT | AUG codon | 93±7.8 |
| TJU-0981 | 4 | GGCACTCACCTCCTCCTTGTT | Ex. 1/In. 1 | 94±7.2 |
| TJU-1534 | 5 | ACACTTACTGAGTGTGAGGGT | Ex. 2/In. 2 | 110±8.5 |
| TJU-1730 | 6 | AAACTTACCTACGACGTGGGC | Ex. 3/In. 3 | 110±9.7 |
| TJU-2431 | 7 | GTCGCCTCACAGAGCAATGAC | Ex. 4/STOP | Activation |
| TJU-2507 | 38 | TAGACGATAAAGGGGTCAGAG | 3′-Untr. Reg. | ca. 115 |
| TJU-2698 | 8 | AGTGAGTTCCGAAAGCCCATT | 3′-Untr. Reg. | 93±8.2 |
| TJU-2719 | 9 | GGCATCGACATTCGGGGATCC | 3′-Untr. Reg. | 85±6.4 |
| TJU-2755 | 10 | TGATCCACTCCCCCCTCCACT | 3′-Untr. Reg. | 7.7±5.1 |
| TJU-2779 | 11 | CAGCCTTGTGAGCCAGAGGCA | 3′-Untr. Reg. | 110±9.4 |
| TJU-2800 | 12 | GGAGGCCTGAGACATCTTCAG | 3′-Untr. Reg. | 100±8.8 |
| TJU-2819 | 13 | AGGGAAGGAAGGAAGGAAGGG | 3′-Untr. Reg. | Activation |
| TJU-2826 | 14 | CTGAGGGAGGGAAGGAAGGAA | 3′-Untr. Reg. | 120±9.8 |
| TJU-2840 | 39 | CAGTCTGGGAAGCTCTGAGGG | 3′-Untr. Reg. | ca. 115 |
| TJU-2879 | 15 | GGTTCCGTAAGGAAGGCTGG | 3′-Untr. Reg. | 93±7.2 |
| TJU-2927 | 16 | AATAATAAATAATAAATAAAT | 3′-Untr. Reg. | 99±8.3 |
| TJU-2991 | 17 | TTCCCAACGCTGGGTCCTCCA | 3′-Untr. Reg. | 98±9.9 |
| TJU-3050 | 40 | GGGATAGCTGGTAGTTTAG | 3′-Untr. Reg. | ca. 100 |
| TJU-3260 | 41 | CATTTCTTTTCCAAGCGAAC | 3′-Untr. Reg. | ca. 90 |
| TJU-3428 | 42 | AGGCTCCTGTTTCCGGGGAGA | 3′-Untr. Reg. | ca. 120 |
| TJU-2734 | 18 | CCCCCGATCCACTCAGGCATC | 3′-Untr. Reg. | 82±6.7 |
| TJU-2737 | 19 | ACTCCCCCGATCCACTCAGGC | 3′-Untr. Reg. | 14±5.3 |
| TJU-2740 | 20 | TCCACTCCCCCGATCCACTCA | 3′-Untr. Reg. | 7.9±4.3 |
| TJU-2743 | 21 | CCCTCCACTCCCCCGATCCAC | 3′-Untr. Reg. | 7.5±4.6 |
| TJU-2746 | 22 | CCCCCCTCCACTCCCCCGATC | 3′-Untr. Reg. | 8.2±4.7 |
| TJU-2749 | 23 | ACTCCCCCCTCCACTCCCCCG | 3′-Untr. Reg. | 9.1±5.4 |
| TJU-2752 | 24 | TCCACTCCCCCCTCCACTCCC | 3′-Untr. Reg. | 7.9±4.4 |
| TJU-2755 | 25 | TGATCCACTCCCCCCTCCACT | 3′-Untr. Reg. | 7.7±5.1 |
| TJU-2758 | 26 | GCCTGATCCACTCCCCCCTCC | 3′-Untr. Reg. | 8.2±4.2 |
| TJU-2761 | 27 | GCAGCCTGATCCACTCCCCCC | 3′-Untr. Reg. | 15±6.2 |
| TJU-2764 | 28 | GAGGCAGCCTGATCCACTCCC | 3′-Untr. Reg. | 98±11 |
| TJU-2755S5 | 29 | AGTGGAGGGGGGAGTGGATCA | | Activation |
| TJU-2755RD | 30 | CCCTCACTGCTACCTCACCTC | | 89±7.0 |
| TJU-2749-19 | 31 | ACTCCCCCCTCCACTCCCC | 3′-Untr. Reg. | 8.6±4.1 |
| TJU-2740-18 | 32 | TCCACTCCCCCGATCCAC | 3′-Untr. Reg. | 7.8±4.0 |
| TJU-2755-16 | 33 | TGATCCACTCCCCCCT | 3′-Untr. Reg. | 8.4±4.3 |

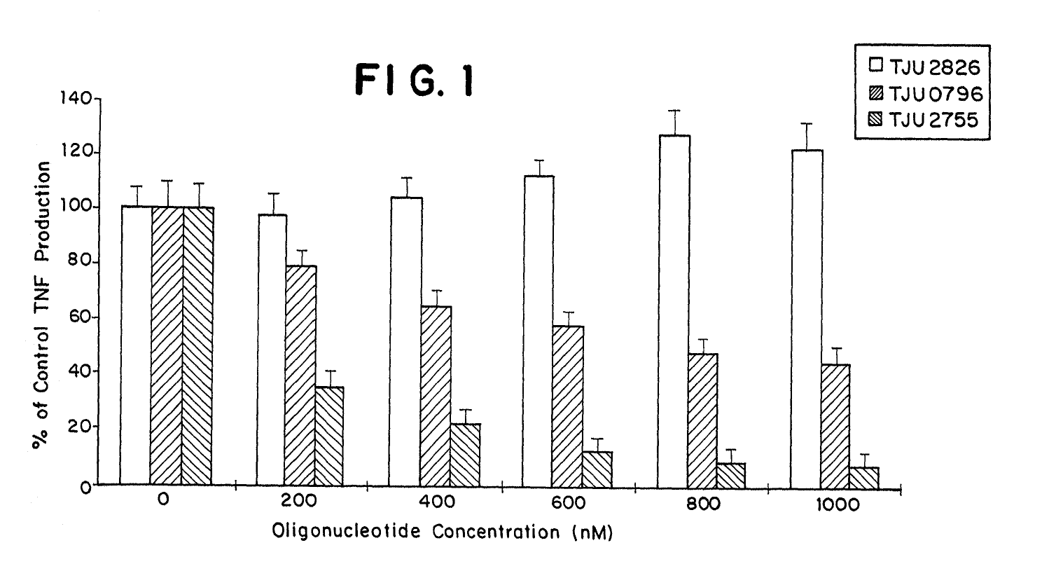

The first twenty-two ASOs listed in Table 2, most of which were selected randomly, and some (i.e., SEQ ID NOs: 38-42) of which were predicted to be efficacious using the methods described herein, were each examined for the ability to inhibit TNF-alpha expression in cultured cells when the ASO was present in the cell culture medium at a concentration of 1 micromolar. Only one of the ASOs, TJU-2755, inhibited the expression of TNF-alpha by at least 90% compared with control cells. TJU-2755 comprised a sequence complementary to a portion of the 3′-untranslated region of the RNA molecule. Another oligonucleotide, TJU-0796, inhibited TNF-alpha expression but with an efficacy of only 50%. The remaining oligonucleotides either had no effect on TNF-alpha expression or actually activated TNF-alpha expression. As indicated in FIG. 1, inhibition of TNF-alpha expression by TJU-2755 is dose-dependent, and the value of 150, the concentration of TJU-2755 in the culture medium which was necessary to effect 50% inhibition of TNF-alpha expression, was approximately 0.1 micromolar.

To test the specificity of TJU-2755 inhibition of TNF-alpha expression, two control oligonucleotides were examined at a concentration of 2 micromolar in the culture medium. A scrambled oligonucleotide, TJU-2755-RD, having the same nucleotide composition as TJU-2755 but in random order, and a sense oligonucleotide TJU-2755-SS, which was complementary to TJU-2755, were assessed for the ability to inhibit TNF-alpha expression. Neither TJU-2755-RD nor TJU-2755-SS inhibited TNF-alpha expression in cultured rat Kupffer cells. This result indicated that the inhibitory effect of TJU-2755 on TNF-alpha expression was markedly dependent on the nucleotide sequence of TJU-2755.

Ten additional oligonucleotides were designed and synthesized, each of which comprised a TCCC motif. The ability of each of these ASOs (SEQ ID NO: 18 through SEQ ID NO: 26 and SEQ ID NO: 28 in Table 2) to inhibit TNF-alpha expression was assessed as described herein. It was determined that all ASOs which inhibited TNF-alpha expression by at least about 80% comprised at least one full TCCC motif (Table 2). The data also establish that ASOs comprising a TCCC motif can comprise fewer than twenty-one, and as few as sixteen or fewer, nucleotide residues (e.g., TJU-2749-19, TJU-2740-18 and TJU-2755-16 in Table 2).

Each of the ASOs indicated in Table 2 which inhibited TNF-alpha expression comprised a TCCC motif and was complementary to an RNA molecule encoding rat TNF-alpha. This demonstrates that a TNF-alpha-specific ASO can be designed by designing an ASO including a TCCC motif and flanking TNF-alpha nucleotide sequence(s). Although only TNF-alpha-specific ASOs comprising between sixteen and twenty-one nucleotide residues have been described herein, it is clear, given the data presented herein, that TNF-alpha-specific ASOs may be designed which comprise more than twenty-one or fewer than sixteen nucleotide residues by including a TCCC motif and at least one flanking TNF-alpha nucleotide sequence in the ASO. Preferably, such ASOs comprise no more than one nucleotide residue which is not complementary to a TNF-alpha-encoding RNA molecule. The ability of these oligonucleotides to inhibit TNF-alpha expression may be easily assessed using the screening methods described herein.

A number of mechanisms are known by which ASOs are capable of inhibiting protein expression, including translational arrest, inhibition of RNA processing, and promotion of RNase H-mediated degradation of the RNA component of the RNA-oligonucleotide complex (Crook, 1993, FEBS J. 7:533-539). A DNA-RNA heteroduplex of 4 to 6 nucleotides in length is sufficient to evoke RNase H activity (Kramer et al., 1984, Cell 38:299-307). Bennett et al. (1991, J. Immunol. 152:3530-3540) demonstrated that ASOs inhibited human ICAM-1 and E-selectin gene expression by two distinct mechanisms. Oligonucleotides which were complementary to the 3′-untranslated region of either gene (ISIS 1939, ISIS 2302, and ISIS 4730) reduced the corresponding mRNA levels, which suggested that RNase H-mediated degradation mechanism was responsible for the inhibition of gene expression. Oligonucleotides which were complementary to region around the AUG translation initiation codon (ISIS 1750 and ISIS 2679) did not alter mRNA levels, which suggested that translational arrest was responsible for the inhibition of gene expression.

### Example 4

The Presence of the TCCC Motif in Reported Efficacious ASOs

A comprehensive search was conducted using the MEDLINE database, current through September 1997, to identify efficacious ASOs which had been reported in the literature. These sequences were examined to determine whether a higher proportion of the sequences comprised a TCCC motif than would be expected by random occurrence of these motifs.

For this literature search, the following conditions were imposed. Only ASOs selected from among 10 or more ASOs as being effective were included. Only ASOs selected from among ASOs designed to target a broad range of RNA regions were included in the search. ASOs presently in FDA-approved human clinical trials were also included in the search.

A total of 42 ASOs complied with these conditions. A TCCC motif was identified in 20 of these ASOs (48%). The nucleotide sequences of the most effective known ASOs comprising the TCCC motif are listed in Table 3. Chi-square analysis indicates that the probability of one TCCC motif existing by chance in 20 of 42 ASOs is remote (p<<0.001; chi²=34.8). By comparison, a VCCC motif (i.e., V is A, G, or C, but not T), the sequence was only found in 5 of the 42 most effective ASO sequences. In only two effective known ASOs was the TCCC motif located at the end of the ASO. Thus, it appears that the TCCC motif should be flanked on both sides by non-TCCC motif nucleotide residues that are complementary to nucleotide sequences which flank the GGGA motif of the corresponding RNA molecule.

In Table 3, 20 of the 42 most efficacious ASOs which have been reported in the literature are listed. Each of these ASOs comprises a TCCC motif. The ASOs are grouped according to the nucleotide residue at the 3′-end of the TCCC motif. For each of the ASOs listed, the identifier used in the reported study is indicated, and the reference number corresponding to the study is listed in parentheses beneath the identifier. A list of citations follows the table. The TCCC motif is underlined in each sequence listing. “mRNA” refers to the region of the corresponding mRNA molecule to which the indicated ASO reported in the study was complementary, and indicates the species and protein corresponding to the mRNA molecule. Where known, the region of the mRNA molecule to which the indicated ASO was complementary is indicated parenthetically. “3′-UTR” refers to the 3′-untranslated region of the mRNA molecule. “AUG” refers to a region comprising the AUG translation initiation codon of the mRNA molecule. “Stop codon” refers to a region comprising a translation stop codon of the mRNA molecule. “5′-UTR” refers to the 5′-untranslated region of the mRNA molecule. “Efficacy” refers to the approximate degree to which gene expression was inhibited in the study. Where only data corresponding to mRNA levels are reported in the indicated study, “M.E.” refers to the oligonucleotide of the study which had the maximum effect. “# tested” refers to the number of oligonucleotides which were compared in the indicated study. “ICAM” means intercellular adhesion molecule. “VCAM” means vascular cell adhesion molecule. “PKC” means protein kinase C. “PAI” means plasminogen activator inhibitor. “NGF” means nerve growth factor. “Xklp” means Xenopus kinesin-like protein. HCV means the 5′-untranslated region of HCV.

TABLE 3

| | | | | |
|---|---|---|---|---|
| Identifier | | Inhibitory Oligonucleotide | | |
| (Ref H) | mRNA | Sequence (listed 5′-3′) | # tested | SEQ ID NO |

A. ASOs comprising a TCCC motif, followed by C:

| | | | | |
|---|---|---|---|---|
| OL(l)p531 | Human p53 (ORF) | CCTGCTCCCCCCTGGCTCC | human trials | 35 |
| ISIS 1939^{2, 3} | Human ICAM-1 (3′-UTR) | CCCCCACCACTTCCCCTCTC | 45 | 36 |
| GM 1508⁴ | Human ICAM-1 (3′-UTR) | CCCCCACCACTTCCCCTCTCA | 39 | 37 |
| ISIS 4189⁵ | Murine PKC-alpha (AUG) | CAGCCATGGTTCCCCCCAAC | 20 | 66 |
| ISIS 4730² | Human E-selectin (3′-UTR) | TTCCCCAGATGCACCTGTTT | 18 | 67 |
| ISIS 11300⁶ | Rat PKC-alpha (ORE) | GACATCCCTTTCCCCCTCGG | 13 | 68 |
| C15⁷ | 1.19CAT (5′-UTR) | GATCCCCGGGTACCGA | 13 | 69 |
| ISIS 3890⁸ | Human PKC-α (AUG) | GTCAGCCATGGTCCCCCCCC | 20 | 70 |
| Oligo 7⁹ | Xenopus Xk1p-1 | ATGCCCTCATCCTTCCCCCCAT | >9 | 71 |

B. ASOs comprising a TCCC motif, followed by A:

| | | | | |
|---|---|---|---|---|
| G 3139¹⁰ | Human bc1-2 (ORF) | GTTCTCCCAGCGTGTGCCAT | human trials | 72 |
| GM 1534⁴ | Human VCAM-1 (5′-UTR) | AACCCTTATTTGTGTCCCACC | 28 | 73 |
| ODN 2309¹¹ | Murine tPA (5′-UTR) | GTCCCAAGAGTTGAGGAG | 18 | 74 |
| ISIS 3466¹² | Human p120 (3′-UTR) | CACCCGCCTTGGCCTCCCAC | 18 | 75 |

C. ASOs comprising a TCCC motif, followed by G:

| | | | | |
|---|---|---|---|---|
| ISIS 5132¹³ | Human C-raf | TCCCGCCTGTGACATGCATT | human trials | 76 |
| ISIS 5995¹⁴ | Human MDR-1 (AUG) | CCATCCCGACCTCGCGCT | 32 | 77 |
| T 195¹⁵ | Human TNF (ORF) | CCACGTCCCGGATCATGC | 15 | 78 |

D. ASOs comprising a TCCC motif, followed by T:

| | | | | |
|---|---|---|---|---|
| 4484-4503¹⁶ | Human HIV (SA) | TCTGCTGTCCCTGTAATAAA | 20 | 79 |
| ISIS 3801³ | Human VCAM | AACCCAGTGCTCCCTTTGCT | 15 | 80 |

E. ASO comprising a TCCC motif at the 3′-end thereof:

| | | | | |
|---|---|---|---|---|
| ISIS 3522¹⁷ | HumanPKC-alpha (AUG) | AAAACGTCAGCCATGGTCCC | 20 | 81 |

### THE REFERENCES INDICATED IN TABLE 3 ARE:

¹Bishop et al., 1996, J. Clin. Oncol. 14:1320-1326

²Chiang et al., 1991, J. Biol. Chem. 266:18162-18171

³Bennett et al., 1994, J. Immunol. 152:3530-3540

⁴Lee et al., 1995, Shock 4:1-10

⁵Dean et al., 1994, Proc. Natl. Acad. Sci. U.S.A. 91:11762-11766

⁶Dean et al., 1996, Biochem. Soc. Trans. 24:623-629

⁷Johansson et al., 1994, Nucl. Acids Res. 22:4591-4598

⁸Dean et al., 1994, J. Biol. Chem. 269:16146-16424

⁹Vemos et al., 1995 Cell 81:117-127

¹⁰Cotter et al., 1994, Oncogene 9:3049-3055

¹¹Stutz et al., 1997, Mol. Cell. Biol. 17:1759-1767

¹²Perlaky et al.,1993, Anti-cancer Drug Des. 8:3-14

¹³Monia et al., 1996, Nature Med. 2:668-675

¹⁴Alahari et al., 1996, Mol. Pharmacol. 50:808-819

¹⁵d'Hellencourt et al., 1996, Biochim. Biophys. Acta 1317:168-174

¹⁶Goodchild et al., 1988, Proc. Natl. Acad. Sci. USA 85:5507-5511

¹⁷Dean et al., 1994, J. Biol. Chem. 269:16416-16424

Hence, it is clear that 5′-TCCC-3′ is a nucleotide motif which confers surprising efficacy on ASOs which comprise the sequence. Because it is well known in the art that uridine has nucleotide binding properties analogous to those of thymidine, one of skill in the art will recognize that T may also be U.

Therefore, it has been demonstrated herein that ASOs which are efficacious for inhibiting expression of genes comprising a corresponding RNA molecule may be made by selecting an ASO comprising a nucleotide sequence which comprises a TCCC motif. That is, ASOs which are efficacious for inhibiting expression of genes comprising a corresponding RNA molecule may be made by selecting an ASO comprising a nucleotide sequence complementary to a region of the corresponding RNA molecule, wherein the region comprises a GGGA motif. Preferably, the TCCC motif is flanked on both sides by nucleotide sequences which are complementary to the corresponding RNA molecule.

It is significant that the efficacy of ASOs which comprise a TCCC motif has been demonstrated in numerous animal species, including rat (described herein), human, mouse, chicken, and toad (each described in studies summarized in Table 3). The skilled artisan will recognize that because no significant difference exists among animals, and particularly between vertebrates, in the ability of an ASO to undergo hybridization in a cell of an animal, the methods and compositions described herein are equally applicable in all animal species.

### Example 5

Prospective Design of ASOs Which are Efficacious for Inhibiting TNF-alpha Expression

In this Example, a series of ASOs were designed to target each of the GGGA motifs identified in an RNA molecule encoding rat TNF-alpha. Based on the published sequence of the rat TNF-alpha gene (Shirai et al., 1989, Agric. Biol. Chem. 53:1733-1736), twenty-eight GGGA motifs exist in the region of the primary transcript of this gene which is located on the 5′-side of the AATAAA polyadenylation site and another three GGGA motifs exist in the region of the primary transcript which is located on the 3′-side of that site. These motifs are located in introns 1-3, in exon 4, and in both the 5′-and 3′-untranslated regions. None of the motifs are located in exon 1, exon 2, or exon 3 of the rat TNF-alpha gene.

The nucleotides sequences of the twenty-five ASOs which were used in this Example are listed in Table 4, which also lists the sequences of ASOs designated TJU-2740 and TJU-2755, which are described elsewhere herein. Among these ASOs, six were designed to be complementary to TNF-alpha-encoding RNA regions comprising either two flanking GGGA motifs or two GGGA motifs comprising no more than six nucleotide residues interposed therebetween. The other ASOs were designed to be complementary to TNF-alpha-encoding RNA regions comprising only one GGGA.

TABLE 4

| Name of ASO | Nucleotide Sequence (5′ to 3′) | SEQ ID NO: | Tₘ, ° C. |
|---|---|---|---|
| TJU-0656 | CTGGTCCCTTGGTGTCCTCGC | 43 | 60.2 |
| TJU-0675 | TTGCTGTTCTCCCTCCTGGCT | 44 | 56.3 |
| TJU-1032 | TTCTTGCCCTCCCTCCCTACT | 45 | 56.3 |
| TJU-1056 | CCTCTTTCCCTTACCCTCCTG | 46 | 56.3 |
| TJU-1103 | GGTCTCCCTCCCCAACTCTCC | 47 | 60.2 |
| TJU-1227 | CTTCTTCCCTGTTCCCCTGGC | 48 | 58.3 |
| TJU-1271 | TATCTCCCTCGTCTCCCATCT | 49 | 54.4 |
| TJU-1310 | GTTTCCCCTCCATCTCCCTCC | 50 | 58.3 |
| TJU-1424 | GAAGCCTCCCCGCTCTTTGCC | 51 | 60.2 |
| TJU-1585 | AAAGCTTTAAGTCCCCCGCCC | 52 | 56.3 |
| TJU-1608 | CCTATTCCCTTTCCTCCCAAA | 53 | 52.4 |
| TJU-1646 | CCCTTAGGTTTCCCAGCAAGC | 54 | 56.3 |
| TJU-1906 | CTGGTCTTTCCACGTCCCATT | 55 | 54.4 |
| TJU-2161 | GCAGCCTTGTCCCTTGAAGAG | 56 | 56.3 |
| TJU-2287 | CTTGAGCTCAGCTCCCTCAGG | 57 | 58.3 |
| TJU-2327 | GCTGGAAGACTCCTCCCAGGT | 58 | 58.3 |
| TJU-2350 | GCTGAGCAGGTCCCCCTTCTC | 59 | 60.2 |
| TJU-2561 | AGAGCCACAATTCCCTTTCTA | 60 | 50.5 |
| TJU-2740 | TCCACTCCCCCGATCCACTCA | 20 | 58.3 |
| TJU-2755 | TGATCCACTCCCCCCTCCACT | 10 | 58.3 |
| TJU-3004 | GCCTGAAGACAGCTTCCCAAC | 61 | 56.3 |
| TJU-3208 | CAGTCACGGCTCCCGTGGG | 62 | 59.7 |
| TJU-3466 | GGGAAATTCCCAGGACCAGGG | 63 | 58.3 |
| TJU-3484 | ATTTGGAATTCCCAGAGTGGG | 64 | 52.4 |
| TJU-3499 | ACTTTCCCAGCAGGTATTTGG | 65 | 52.4 |

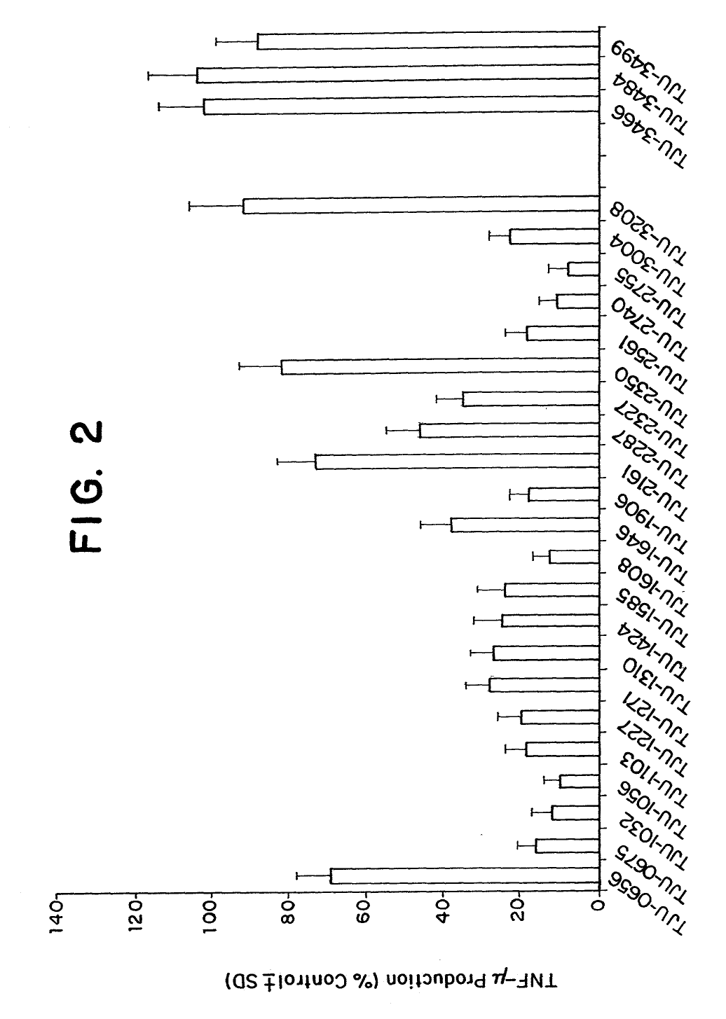

The ability of the ASOs described in this Example to inhibit TNF-alpha expression was assessed as described herein using an ASO concentration of 1 micromolar. As indicated in FIG. 2, more than half (13/22) of the ASOs described in this Example inhibited TNF-alpha expression by 75% or more. Seven of the ASOs described in this Example did not significantly inhibit TNF-alpha expression, including all three of the ASOs designed to be complementary to a GGGA motif located on the 3′-side of the AATAAA polyadenylation site of TNF-alpha-encoding RNA.

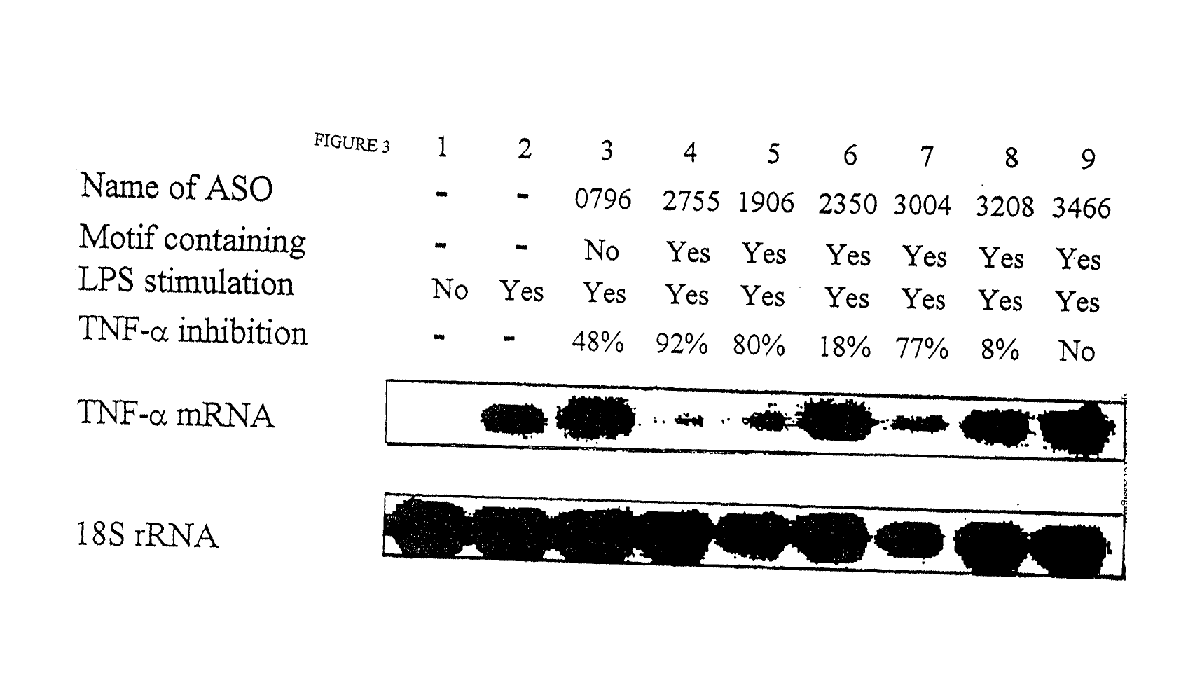

The effect of the presence of several of the ASOs described in this Example on the steady-state level of mRNA encoding TNF-alpha was assessed by Northern analysis of RNA obtained from cells cultured in the presence of these ASOs. The results of these Northern analyses are summarized in FIG. 3. It is evident in FIG. 3 that levels of mRNA encoding TNF-alpha were depressed in cells which were cultured in the presence of ASOs which inhibited expression of TNF-alpha (i.e., lanes 4, 5, and 7, corresponding to cells cultured in the presence of TJU-2755, TJU-1906, and TJU-3004, respectively). Levels of 18S RNA were unaffected.

Without wishing to be bound by any particular theory, it is hypothesized that these results indicate that inhibition of TNF-alpha expression by these ASOs occurs by a mechanism which promotes degradation of RNA molecules encoding TNF-alpha. The fact that TNF-alpha-inhibitory ASOs were complementary to regions of the primary transcript comprising a GGGA motif suggests that the expression-inhibiting effect is exerted in the nucleus, before the primary transcript is spliced. This hypothesis is consistent with reports that ASOs rapidly accumulate in the nucleus after being introduced directly into the cell cytoplasm by microinjection, electroporation, streptolysin O treatment, or cationic liposome delivery (Giles et al., 1995, Antisense Res. Dev. 5:23-31).

Therefore, while remaining not bound by any particular theory of operation, it is hypothesized that ASOs, RNases and newly synthesized RNA molecules are present in the nucleus following delivery of an ASO to a cell. The nucleus is the primary site at which ASOs exert their gene-expression-inhibiting effect. The primary transcript of the expression-inhibited gene is the physiological target with which the ASO interacts, rather than the mature mRNA corresponding to that gene. It may be that the mechanism by which an ASO effects primary transcript degradation involves a nuclear RNase.

According to this hypothesis, RNA regions comprising an GGGA motif may be preferred sites for RNase digestion. This hypothesis is supported by the observations of Lima and Crooke (1997, Biochemistry 36:390-398), which indicated that although RNase H was not highly specific with regard to the nucleotide sequence of the DNA-RNA hybrid on which it acts, it preferentially bound to the A-form of a DNA-RNA duplex. Since RNA sequences containing high purine content are predicted to stack in the A-form conformation (Ratmeyer et al., 1994, Biochemistry 33:5298-5304), RNase H activity may be improved using ASOs containing pyrimidine-rich sequences (i.e., which are complementary to RNA molecules which have purine-rich sequences and which therefore are likely to assume the A-form conformation). As can be ascertained by reviewing the nucleotide sequences listed in Table 4, both the TCCC motif itself and the bases at either end of the motif are pyrimidine-rich in the most potent ASOs.

This hypothesis may help explain why many ASOs designed by others were not efficacious. Most of ASOs reported in the literature were designed to target a region of a mature mRNA molecule, rather than a region of the corresponding primary transcript. For example, about 70% of reported ASOs were designed to target the mRNA region comprising the AUG codon. Were these ASOs instead designed to be complementary to a region of the primary transcript, particularly a region comprising a GGGA motif as described herein, these ASOs might have been more efficacious.

The experiments described in this Example demonstrate that, in contrast to empirical screening, designing ASOs by targeting the fragments comprising a GGGA motif, as described in this Example, is much more likely to yield ASO sequences which are efficacious for inhibiting expression of a gene product.

### Example 6

ASOs Which are Efficacious for Inhibiting Expression of Proteins Other than TNF-Alpha

The inventors have used the strategy described herein to design ASOs which were efficacious for inhibiting expression of genes other than TNF-alpha. By way of example, a successful design of ASOs efficacious to inhibit expression of rat inducible nitric oxide synthase was described by Cao et al. (1998, Alcoholism Clin. Exp. Res. 22:108a).

The disclosures of each and every patent, patent application and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An antisense oligonucleotide for inhibiting expression of an aldehyde dehydrogenase gene in a cell, the oligonucleotide comprising at least 12 nucleotide residues and having a sequence selected such that the oligonucleotide anneals in the cell with a portion of an RNA molecule corresponding to the gene, wherein the portion comprises a GGGA motif, whereby the oligonucleotide inhibits expression of the gene in the cell.

2. The oligonucleotide of claim 1, wherein the gene is an ALDH2 gene.

3. The oligonucleotide of claim 2, wherein the gene is a human gene.

4. The oligonucleotide of claim 3, wherein the RNA molecule corresponds to the ALDH2-1 allele of the ALDH2 gene.

5. The oligonucleotide of claim 3, wherein the sequence of the oligonucleotide is homologous with or complementary to at least 12 nucleotide residues of one of SEQ ID NOs: 108 and 110.

6. The oligonucleotide of claim 1, wherein the oligonucleotide comprises from 12 to 2000 nucleotide residues.

7. The oligonucleotide of claim 1, wherein the oligonucleotide comprises from 12 to 50 nucleotide residues.

8. The oligonucleotide of claim 7, wherein the oligonucleotide comprises from 14 to 30 nucleotide residues.

9. The oligonucleotide of claim 7, wherein the oligonucleotide comprises from 16 to 23 nucleotide residues.

10. The oligonucleotide of claim 1, wherein the oligonucleotide is completely complementary to the portion.

11. The oligonucleotide of claim 1, wherein the oligonucleotide is at least 90% complementary to the portion.

12. The oligonucleotide of claim 1, wherein the oligonucleotide is at least 95% complementary to the portion.

13. The oligonucleotide of claim 1, wherein the sequence of the oligonucleotide comprises a sequence selected from the group consisting of SEQ ID NOs: 98, 107, 109, and 111.

14. The oligonucleotide of claim 13, having the sequence SEQ ID NO: 98.

15. The oligonucleotide of claim 13, having the sequence SEQ ID NO: 111.

16. The oligonucleotide of claim 1, wherein the RNA molecule is the primary transcript of the gene.

17. The oligonucleotide of claim 1, wherein the RNA molecule is an mRNA of the gene.

18. The oligonucleotide of claim 1, wherein at least one linkage between the nucleotide residues of the oligonucleotide is a non-phosphodiester linkage.

19. The oligonucleotide of claim 18, wherein at least one linkage between nucleotide residues of the oligonucleotide is a phosphorothioate linkage.

20. The oligonucleotide of claim 18, wherein every linkage between nucleotide residues of the oligonucleotide is a phosphorothioate linkage.

21. A pharmaceutical composition for inhibiting expression of an aldehyde dehydrogenase gene in a cell, the composition comprising the oligonucleotide of claim 1 suspended in a pharmaceutically acceptable carrier.

22. A pharmaceutical composition for inhibiting expression of an aldehyde dehydrogenase gene in a cell, the pharmaceutical composition comprising a transcription vector for transcribing the oligonucleotide of claim 1 in the cell, the vector being suspended in a pharmaceutically acceptable carrier.

23. The method of claim 22, wherein the transcription vector is a plasmid.

24. The method of claim 22, wherein the transcription vector is a virus vector.

25. A method of decreasing ethanol tolerance in a human, the method comprising administering the oligonucleotide of claim 1 to liver cells of the human, whereby the oligonucleotide inhibits expression of the aldehyde dehydrogenase gene in the cells and decreases ethanol tolerance in the human.

26. The method of claim 25, wherein the oligonucleotide is administered to the cells by delivering the oligonucleotide to the bloodstream of the human.

27. The method of claim 25, wherein the oligonucleotide is administered to the cells by injecting a pharmaceutical composition comprising the oligonucleotide into the liver of the human.

28. The method of claim 25, wherein the oligonucleotide is administered to the cells by administering to the cells a transcription vector for transcribing the oligonucleotide in the cells.

29. The method of claim 28, wherein the transcription vector is a plasmid.

30. The method of claim 28, wherein the transcription vector is a virus vector.

31. A method of inhibiting ethanol intake by a human, the method comprising administering the oligonucleotide of claim 1 to liver cells of the human, whereby the oligonucleotide inhibits expression of the aldehyde dehydrogenase gene in the cells, decreases the ethanol tolerance of the human, and thereby inhibits ethanol intake by the human.

32. A method of decreasing the desire of a human to consume ethanol, the method comprising administering the oligonucleotide of claim 1 to liver cells of the human, whereby the oligonucleotide inhibits expression of the aldehyde dehydrogenase gene in the cells, thereby decreasing the ability of the human to metabolize acetaldehyde and increasing the non-desirability of ethanol consumption.

33. A polynucleotide for inhibiting aldehyde dehydrogenase activity in a cell, the polynucleotide encoding an exogenous ALDH2-2 allele operably linked with a promoter/regulatory region, whereby the ALDH2-2 allele is expressed in the cell when the polynucleotide is delivered to the interior of the cell, the ALDH2-2 gene product multimerizes with an endogenous aldehyde dehydrogenase subunit, and the activity of the endogenous aldehyde dehydrogenase is inhibited.

34. The polynucleotide of claim 33, wherein the cell is a human cell.

35. The polynucleotide of claim 34, wherein the cell is a liver cell.

36. The polynucleotide of claim 33, wherein the exogenous ALDH2-2 allele has the nucleotide sequence SEQ ID NO: 110 having an adenine residue at position 1543.

37. The polynucleotide of claim 33, wherein the exogenous ALDH2-2 allele is operably linked with an expression vector.

38. The method of claim 37, wherein the expression vector is a plasmid.

39. The method of claim 37, wherein the expression vector is a virus vector.

40. A pharmaceutical composition for inhibiting aldehyde dehydrogenase activity in a cell, the pharmaceutical composition comprising the polynucleotide of claim 33 suspended in a pharmaceutically acceptable carrier.

41. A method of decreasing ethanol tolerance in a human, the method comprising administering the polynucleotide of claim 33 to liver cells of the human, whereby the ALDH2-2 allele is expressed in the cells, aldehyde dehydrogenase activity is inhibited in the cells, and ethanol tolerance decreases in the human.

42. A method of inhibiting ethanol intake by a human, the method comprising administering the polynucleotide of claim 33 to liver cells of the human, whereby the ALDH2-2 allele is expressed and inhibits aldehyde dehydrogenase activity in the cells, the ethanol tolerance of the human decreases, and ethanol intake by the human is thereby inhibited.

43. A method of decreasing the desire of a human to consume ethanol, the method comprising administering the polynucleotide of claim 33 to liver cells of the human, whereby the ALDH2-2 allele is expressed and inhibits aldehyde dehydrogenase activity in the cells, thereby decreasing the ability of the human to metabolize acetaldehyde and increasing the non-desirability of ethanol consumption.

44. A method of making an antisense oligonucleotide for inhibiting aldehyde dehydrogenase activity in a cell of a mammal, the method comprising
selecting a portion of an RNA molecule corresponding to an allele of the gene encoding the aldehyde dehydrogenase, wherein the portion comprises a GGGA motif and
synthesizing an oligonucleotide comprising at least 12 nucleotide residues and having a sequence selected such that it anneals in the cell with the portion,
whereby the oligonucleotide inhibits expression of the gene when it is administered to the interior of the cell.

45. An antisense oligonucleotide for inhibiting expression of a gene which encodes TNF-alpha in an animal, the oligonucleotide comprising from 12 to 50 nucleotide residues, wherein at least 90% of the nucleotide residues of the oligonucleotide are complementary to a region of an RNA molecule which corresponds to the gene, wherein the region comprises a GGGA motif.

46. The antisense oligonucleotide of claim 45, wherein the oligonucleotide comprises from 14 to 30 nucleotide residues, wherein the oligonucleotide comprises a TCCC motif, and wherein at least 95% of the nucleotide residues of the oligonucleotide are complementary to the region.

47. The antisense oligonucleotide of claim 45, wherein the oligonucleotide comprises from 16 to 21 nucleotide residues, comprises a TCCC motif, and is completely complementary to the region.

48. The antisense oligonucleotide of claim 45, wherein the animal is a human.

49. The antisense oligonucleotide of claim 48, wherein the antisense oligonucleotide is complementary to from 12 to 50 consecutive nucleotide residues of a region of the human TNF-alpha gene selected from the group consisting of regions I-XXII, the antisense oligonucleotide being complementary to at least one GGGA motif in the region.

50. An antisense oligonucleotide for inhibiting expression of a gene in an animal cell, the oligonucleotide being made by identifying an RNA molecule corresponding to the gene, wherein the RNA molecule comprises a GGGA motif; and
synthesizing an oligonucleotide complementary to at least a portion of the RNA molecule, the portion comprising the motif,
whereby the oligonucleotide inhibits expression of the gene when it is administered to the interior of the cell.

51. A method of treating an animal afflicted with a disease or disorder characterized by the presence in an affected cell of the animal of an RNA molecule which corresponds to a gene, which RNA molecule comprises a region comprising a GGGA motif, the method comprising
providing an antisense oligonucleotide which is at least 90% complementary to the region; and
administering the oligonucleotide to the animal.

52. The method of claim 51, wherein the antisense oligonucleotide is completely complementary to the region.

53. The method of claim 51, wherein the RNA molecule is the primary transcript of the gene.

54. The method of claim 51, wherein the animal is a human.

55. The method of claim 54, wherein the gene encodes human TNF-alpha.

56. The method of claim 51, wherein at least one linkage between nucleotide residues of the oligonucleotide is a phosphorothioate linkage.

57. A method of inhibiting expression of a gene in an animal cell, the method comprising administering to the cell an antisense oligonucleotide which is complementary to a region of an RNA molecule corresponding to the gene, wherein the region comprises a GGGA motif.

58. A method of predicting the efficacy of an antisense oligonucleotide for inhibiting expression of a gene, the method comprising determining whether the antisense oligonucleotide is complementary to a region of an RNA molecule corresponding to the gene, wherein the region comprises a GGGA motif, whereby complementarity of the antisense oligonucleotide to the portion is an indication that the antisense oligonucleotide is efficacious for inhibiting expression of the gene.

59. A method of separating from a mixture of oligonucleotides an antisense oligonucleotide which is efficacious for inhibiting expression of a gene, the method comprising
contacting the mixture with a support linked to an oligonucleotide comprising a GGGA motif, whereby the efficacious antisense oligonucleotide associates with the support; and
separating the support from the mixture.


SEQUENCE LISTING (111 sequences)

SEQ ID NO: 1 - DNA, 21 bp, Artificial Sequence
  Description of Artificial Sequence Candidate TNF(alpha) ASO
cctcgctgagttctgccggct

SEQ ID NO: 2 - DNA, 21 bp, Artificial Sequence
  Description of Artificial Sequence Candidate TNF(alpha) ASO
ccgtgctcatggtgtcctttc

SEQ ID NO: 3 - DNA, 21 bp, Artificial Sequence
  Description of Artificial Sequence Candidate TNF(alpha) ASO
gatcatgctttccgtgctcat

SEQ ID NO: 4 - DNA, 21 bp, Artificial Sequence
  Description of Artificial Sequence Candidate TNF(alpha) ASO
ggcactcacctcctccttgtt

SEQ ID NO: 5 - DNA, 21 bp, Artificial Sequence
  Description of Artificial Sequence Candidate TNF(alpha) ASO
acacttactgagtgtgagggt

SEQ ID NO: 6 - DNA, 21 bp, Artificial Sequence
  Description of Artificial Sequence Candidate TNF(alpha) ASO
aaacttacctacgacgtgggc

SEQ ID NO: 7 - DNA, 21 bp, Artificial Sequence
  Description of Artificial Sequence Candidate TNF(alpha) ASO
gtcgcctcacagagcaatgac

SEQ ID NO: 8 - DNA, 21 bp, Artificial Sequence
  Description of Artificial Sequence Candidate TNF(alpha) ASO
agtgagttccgaaagcccatt

SEQ ID NO: 9 - DNA, 21 bp, Artificial Sequence
  Description of Artificial Sequence Candidate TNF(alpha) ASO
ggcatcgacattcggggatcc

SEQ ID NO: 10 - DNA, 21 bp, Artificial Sequence
  Description of Artificial Sequence Candidate TNF(alpha) ASO
tgatccactcccccctccact

SEQ ID NO: 11 - DNA, 21 bp, Artificial Sequence
  Description of Artificial Sequence Candidate TNF(alpha) ASO
cagccttgtgagccagaggca

SEQ ID NO: 12 - DNA, 21 bp, Artificial Sequence
  Description of Artificial Sequence Candidate TNF(alpha) ASO
ggaggcctgagacatcttcag

SEQ ID NO: 13 - DNA, 21 bp, Artificial Sequence
  Description of Artificial Sequence Candidate TNF(alpha) ASO
agggaaggaaggaaggaaggg

SEQ ID NO: 14 - DNA, 21 bp, Artificial Sequence
  Description of Artificial Sequence Candidate TNF(alpha) ASO
ctgagggagggaaggaaggaa

SEQ ID NO: 15 - DNA, 20 bp, Artificial Sequence
  Description of Artificial Sequence Candidate TNF(alpha) ASO
ggttccgtaaggaaggctgg

SEQ ID NO: 16 - DNA, 21 bp, Artificial Sequence
  Description of Artificial Sequence Candidate TNF(alpha) ASO
aataataaataataaataaat

SEQ ID NO: 17 - DNA, 21 bp, Artificial Sequence
  Description of Artificial Sequence Candidate TNF(alpha) ASO
ttcccaacgctgggtcctcca

SEQ ID NO: 18 - DNA, 21 bp, Artificial Sequence
  Description of Artificial Sequence Candidate TNF(alpha) ASO
cccccgatccactcaggcatc

SEQ ID NO: 19 - DNA, 21 bp, Artificial Sequence
  Description of Artificial Sequence Candidate TNF(alpha) ASO
actcccccgatccactcaggc

SEQ ID NO: 20 - DNA, 21 bp, Artificial Sequence
  Description of Artificial Sequence Candidate TNF(alpha) ASO
tccactcccccgatccactca

SEQ ID NO: 21 - DNA, 21 bp, Artificial Sequence
  Description of Artificial Sequence Candidate TNF(alpha) ASO
ccctccactcccccgatccac

SEQ ID NO: 22 - DNA, 21 bp, Artificial Sequence
  Description of Artificial Sequence Candidate TNF(alpha) ASO
cccccctccactcccccgatc

SEQ ID NO: 23 - DNA, 21 bp, Artificial Sequence
  Description of Artificial Sequence Candidate TNF(alpha) ASO
actcccccctccactcccccg

SEQ ID NO: 24 - DNA, 21 bp, Artificial Sequence
  Description of Artificial Sequence Candidate TNF(alpha) ASO
tccactcccccctccactccc

SEQ ID NO: 25 - DNA, 21 bp, Artificial Sequence
  Description of Artificial Sequence Candidate TNF(alpha) ASO
tgatccactcccccctccact

SEQ ID NO: 26 - DNA, 21 bp, Artificial Sequence
  Description of Artificial Sequence Candidate TNF(alpha) ASO
gcctgatccactcccccctcc

SEQ ID NO: 27 - DNA, 21 bp, Artificial Sequence
  Description of Artificial Sequence Candidate TNF(alpha) ASO
gcagcctgatccactcccccc

SEQ ID NO: 28 - DNA, 21 bp, Artificial Sequence
  Description of Artificial Sequence Candidate TNF(alpha) ASO
gaggcagcctgatccactccc

SEQ ID NO: 29 - DNA, 21 bp, Artificial Sequence
  Description of Artificial Sequence Candidate TNF(alpha) ASO
agtggaggggggagtggatca

SEQ ID NO: 30 - DNA, 21 bp, Artificial Sequence
  Description of Artificial Sequence Candidate TNF(alpha) ASO
ccctcactgctacctcacctc

SEQ ID NO: 31 - DNA, 19 bp, Artificial Sequence
  Description of Artificial Sequence Candidate TNF(alpha) ASO
actcccccctccactcccc

SEQ ID NO: 32 - DNA, 18 bp, Artificial Sequence
  Description of Artificial Sequence Candidate TNF(alpha) ASO
tccactcccccgatccac

SEQ ID NO: 33 - DNA, 16 bp, Artificial Sequence
  Description of Artificial Sequence Candidate TNF(alpha) ASO
tgatccactcccccct

SEQ ID NO: 34 - DNA, 3634 bp, Homo sapiens
gaattccgggtgatttcactcccggctgtccaggcttgtcctgctaccccacccagcctttcctgaggcctcaagcctgccaccaagcccccagctccttctccccgcaggacccaaacacaggcctcaggactcaacacagcttttccctccaacccgttttctctccctcaacggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcggagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagccagcagacgctccctcagcaaggacagcagaggaccagctaagagggagagaagcaactacagaccccccctgaaaacaaccctcagacgccacatcccctgacaagctgccaggcaggttctcttcctctcacatactgacccacggcttcaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagaggtgagtgcctggccagccttcatccactctcccacccaaggggaaatgagagacgcaagagagggagagagatgggatgggtgaaagatgtgcgctgatagggagggatgagagagaaaaaaacatggagaaagacggggatgcagaaagagatgtggcaagagatggggaagagagagagagaaagatggagagacaggatgtctggcacatggaaggtgctcactaagtgtgtatggagtgaatgaatgaatgaatgaatgaacaagcagatatataaataagatatggagacagatgtggggtgtgagaagagagatgggggaagaaacaagtgatatgaataaagatggtgagacagaaagagcgggaaatatgacagctaaggagagagatgggggagataaggagagaagaagatagggtgtctggcacacagaagacactcagggaaagagctgttgaatgctggaaggtgaatacacagatgaatggagagagaaaaccagacacctcagggctaagagcgcaggccagacaggcagccagctgttcctcctttaagggtgactccctcgatgttaaccattctccttctccccaacagttccccagggacctctctctaatcagccctctggcccaggcagtcagtaagtgtctccaaacctctttcctaattctgggtttgggtttgggggtagggttagtaccggtatggaagcagtgggggaaatttaaagttttggtcttgggggaggatggatggaggtgaaagtaggggggtattttctaggaagtttaagggtctcagctttttcttttctctctcctcttcaggatcatcttctcgaaccccgagtgacaagcctgtagcccatgttgtaggtaagagctctgaggatgtgtcttggaacttggagggctaggatttggggattgaagcccggctgatggtaggcagaacttggagacaatgtgagaaggactcgctgagctcaagggaagggtggaggaacagcacaggccttagtgggatactcagaacgtcatggccaggtgggatgtgggatgacagacagagaggacaggaaccggatgtggggtgggcagagctcgagggccaggatgtggagagtgaaccgacatggccacactgactctcctctccctctctccctccctccagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaacatggtctccttcttggaattaattctgcatctgcctcttcttgtgggtgggaagaagctccctaagtcctctctccacaggctttaagatccctcggacccagtcccatccttagactcctagggccctggagaccctacataaacaaagcccaacagaatattccccatcccccaggaaacaagagcctgaacctaattacctctccctcagggcatgggaatttccaactctgggaattc

SEQ ID NO: 35 - DNA, 19 bp, Artificial Sequence
  Description of Artificial Sequence Known effective ASO
cctgctcccccctggctcc

SEQ ID NO: 36 - DNA, 20 bp, Artificial Sequence
  Description of Artificial Sequence Known effective ASO
cccccaccacttcccctctc

SEQ ID NO: 37 - DNA, 21 bp, Artificial Sequence
  Description of Artificial Sequence Known effective ASO
cccccaccacttcccctctca

SEQ ID NO: 38 - DNA, 21 bp, Artificial Sequence
  Description of Artificial Sequence Candidate TNF(alpha) ASO
tagacgataaaggggtcagag

SEQ ID NO: 39 - DNA, 21 bp, Artificial Sequence
  Description of Artificial Sequence Candidate TNF(alpha) ASO
cagtctgggaagctctgaggg

SEQ ID NO: 40 - DNA, 19 bp, Artificial Sequence
  Description of Artificial Sequence Candidate TNF(alpha) ASO
gggatagctggtagtttag

SEQ ID NO: 41 - DNA, 20 bp, Artificial Sequence
  Description of Artificial Sequence Candidate TNF(alpha) ASO
catttcttttccaagcgaac

SEQ ID NO: 42 - DNA, 21 bp, Artificial Sequence
  Description of Artificial Sequence Candidate TNF(alpha) ASO
aggctcctgtttccggggaga

SEQ ID NO: 43 - DNA, 21 bp, Artificial Sequence
  Description of Artificial Sequence Candidate TNF(alpha) ASO
ctggtcccttggtgtcctcgc

SEQ ID NO: 44 - DNA, 21 bp, Artificial Sequence
  Description of Artificial Sequence Candidate TNF(alpha) ASO
ttgctgttctccctcctggct

SEQ ID NO: 45 - DNA, 21 bp, Artificial Sequence
  Description of Artificial Sequence Candidate TNF(alpha) ASO
ttcttgccctccctccctact

SEQ ID NO: 46 - DNA, 21 bp, Artificial Sequence
  Description of Artificial Sequence Candidate TNF(alpha) ASO
cctctttcccttaccctcctg

SEQ ID NO: 47 - DNA, 21 bp, Artificial Sequence
  Description of Artificial Sequence Candidate TNF(alpha) ASO
ggtctccctccccaactctcc

SEQ ID NO: 48 - DNA, 21 bp, Artificial Sequence
  Description of Artificial Sequence Candidate TNF(alpha) ASO
cttcttccctgttcccctggc

SEQ ID NO: 49 - DNA, 21 bp, Artificial Sequence
  Description of Artificial Sequence Candidate TNF(alpha) ASO
tatctccctcgtctcccatct

SEQ ID NO: 50 - DNA, 21 bp, Artificial Sequence
  Description of Artificial Sequence Candidate TNF(alpha) ASO
gtttcccctccatctccctcc

SEQ ID NO: 51 - DNA, 21 bp, Artificial Sequence
  Description of Artificial Sequence Candidate TNF(alpha) ASO
gaagcctccccgctctttgcc

SEQ ID NO: 52 - DNA, 21 bp, Artificial Sequence
  Description of Artificial Sequence Candidate TNF(alpha) ASO
aaagctttaagtcccccgccc

SEQ ID NO: 53 - DNA, 21 bp, Artificial Sequence
  Description of Artificial Sequence Candidate TNF(alpha) ASO
cctattccctttcctcccaaa

SEQ ID NO: 54 - DNA, 21 bp, Artificial Sequence
  Description of Artificial Sequence Candidate TNF(alpha) ASO
cccttaggtttcccagcaagc

SEQ ID NO: 55 - DNA, 21 bp, Artificial Sequence
  Description of Artificial Sequence Candidate TNF(alpha) ASO
ctggtctttccacgtcccatt

SEQ ID NO: 56 - DNA, 21 bp, Artificial Sequence
  Description of Artificial Sequence Candidate TNF(alpha) ASO
gcagccttgtcccttgaagag

SEQ ID NO: 57 - DNA, 21 bp, Artificial Sequence
  Description of Artificial Sequence Candidate TNF(alpha) ASO
cttgagctcagctccctcagg

SEQ ID NO: 58 - DNA, 21 bp, Artificial Sequence
  Description of Artificial Sequence Candidate TNF(alpha) ASO
gctggaagactcctcccaggt

SEQ ID NO: 59 - DNA, 21 bp, Artificial Sequence
  Description of Artificial Sequence Candidate TNF(alpha) ASO
gctgagcaggtcccccttctc

SEQ ID NO: 60 - DNA, 21 bp, Artificial Sequence
  Description of Artificial Sequence Candidate TNF(alpha) ASO
agagccacaattccctttcta

SEQ ID NO: 61 - DNA, 21 bp, Artificial Sequence
  Description of Artificial Sequence Candidate TNF(alpha) ASO
gcctgaagacagcttcccaac

SEQ ID NO: 62 - DNA, 19 bp, Artificial Sequence
  Description of Artificial Sequence Candidate TNF(alpha) ASO
cagtcacggctcccgtggg

SEQ ID NO: 63 - DNA, 21 bp, Artificial Sequence
  Description of Artificial Sequence Candidate TNF(alpha) ASO
gggaaattcccaggaccaggg

SEQ ID NO: 64 - DNA, 21 bp, Artificial Sequence
  Description of Artificial Sequence Candidate TNF(alpha) ASO
atttggaattcccagagtggg

SEQ ID NO: 65 - DNA, 21 bp, Artificial Sequence
  Description of Artificial Sequence Candidate TNF(alpha) ASO
actttcccagcaggtatttgg

SEQ ID NO: 66 - DNA, 20 bp, Artificial Sequence
  Description of Artificial Sequence Known effective ASO
cagccatggttccccccaac

SEQ ID NO: 67 - DNA, 20 bp, Artificial Sequence
  Description of Artificial Sequence Known effective ASO
ttccccagatgcacctgttt

SEQ ID NO: 68 - DNA, 20 bp, Artificial Sequence
  Description of Artificial Sequence Known effective ASO
gacatccctttccccctcgg

SEQ ID NO: 69 - DNA, 16 bp, Artificial Sequence
  Description of Artificial Sequence Known effective ASO
gatccccgggtaccga

SEQ ID NO: 70 - DNA, 20 bp, Artificial Sequence
  Description of Artificial Sequence Known effective ASO
gtcagccatggtcccccccc

SEQ ID NO: 71 - DNA, 22 bp, Artificial Sequence
  Description of Artificial Sequence Known effective ASO
atgccctcatccttccccccat

SEQ ID NO: 72 - DNA, 20 bp, Artificial Sequence
  Description of Artificial Sequence Known effective ASO
gttctcccagcgtgtgccat

SEQ ID NO: 73 - DNA, 21 bp, Artificial Sequence
  Description of Artificial Sequence Known effective ASO
aacccttatttgtgtcccacc

SEQ ID NO: 74 - DNA, 18 bp, Artificial Sequence
  Description of Artificial Sequence Known effective ASO
gtcccaagagttgaggag

SEQ ID NO: 75 - DNA, 20 bp, Artificial Sequence
  Description of Artificial Sequence Known effective ASO
cacccgccttggcctcccac

SEQ ID NO: 76 - DNA, 20 bp, Artificial Sequence
  Description of Artificial Sequence Known effective ASO
tcccgcctgtgacatgcatt

SEQ ID NO: 77 - DNA, 18 bp, Artificial Sequence
  Description of Artificial Sequence Known effective ASO
ccatcccgacctcgcgct

SEQ ID NO: 78 - DNA, 18 bp, Artificial Sequence
  Description of Artificial Sequence Known effective ASO
ccacgtcccggatcatgc

SEQ ID NO: 79 - DNA, 20 bp, Artificial Sequence
  Description of Artificial Sequence Known effective ASO
tctgctgtccctgtaataaa

SEQ ID NO: 80 - DNA, 20 bp, Artificial Sequence
  Description of Artificial Sequence Known effective ASO
aacccagtgctccctttgct

SEQ ID NO: 81 - DNA, 20 bp, Artificial Sequence
  Description of Artificial Sequence Known effective ASO
aaaacgtcagccatggtccc

SEQ ID NO: 82-96 - skipped (15 entries)

SEQ ID NO: 97 - DNA, 21 bp, Artificial Sequence
  Description of Artificial Sequence Control oligonucleotide
cagatgacctcccccgtggaa

SEQ ID NO: 98 - DNA, 21 bp, Artificial Sequence
  Description of Artificial Sequence ASO-9
tcctccttgttcccttcggct

SEQ ID NO: 99 - DNA, 21 bp, Artificial Sequence
  Description of Artificial Sequence Control oligonucleotide
cgtcttcacttccgtgtaggc

SEQ ID NO: 100 - DNA, 21 bp, Artificial Sequence
  Description of Artificial Sequence 2-base mismatch of ASO-9
tcctcgttgttcgcttcggct

SEQ ID NO: 101 - DNA, 21 bp, Artificial Sequence
  Description of Artificial Sequence 3-base mismatch of ASO-9
tcctcgttgttcgcatcggct

SEQ ID NO: 102 - DNA, 21 bp, Artificial Sequence
  Description of Artificial Sequence 4-base mismatch of ASO-9
tccacgttgtacgcatcggct

SEQ ID NO: 103-106 - skipped (4 entries)

SEQ ID NO: 107 - DNA, 21 bp, Artificial Sequence
  Description of Artificial Sequence Complement of ASO-9
agccgaagggaacaaggagga

SEQ ID NO: 108 - DNA, 1889 bp, Rattus norvegicus
gctttatctgctaagctccgctcagttcagcatgctgcgcgccgcactcagcaccgcccgccgtgggccacgcctgagccgcctgctgtccgccgccgccaccagcgcggtgccagcccccaaccagcagcccgaggtcttctgcaaccagatcttcattaacaatgagtggcatgatgctgtcagcaagaaaacattccccaccgtcaacccttccacgggggaggtcatctgccaggtagccgaagggaacaaggaggacgtagacaaggcagtgaaggccgctcaggcagccttccagctgggctcgccctggcgccgcatggatgcatctgacaggggccggctgttgtaccgattggctgatctcatcgaacgggaccggacctacctggcggccttggagaccctggacaacggcaagccttatgtcatctcctacctggtggatttggacatggttctgaaatgtctccgctattatgctggctgggctgacaagtaccacgggaaaaccattcccatcgatggcgacttcttcagctacacccgccacgagcctgtgggcgtgtgtggacagatcattccgtggaacttcccgctcctgatgcaagcctggaagctgggccctgccttggcaactggaaacgtggtggtgatgaaagtggccgagcagacaccgctcactgcactctacgtggccaacttgatcaaggaggcaggcttcccccctggtgtggtcaatattgttcctggattcggccctaccgccggggctgccatcgcgtcccacgaggatgtggacaaagtggccttcacaggttccactgaggttggtcacctaatccaggttgccgccgggagcagcaatctcaagagagtaaccctggaactggggggaaagagccccaatatcatcatgtcagacgctgacatggactgggctgtggaacaggcccactttgccctgttcttcaaccagggccagtgctgttgtgcgggctcccggaccttcgtgcaggaggatgtgtatgatgaattcgtggaacgcagtgtggcccgggccaagtctcgggtggtcgggaaccctttcgacagccggacggagcaggggccgcaggtggatgagactcagtttaagaagatcctgggctatatcaagtcaggacaacaagaaggggcgaagctgctgtgcggtgggggcgccgccgcagaccgtggttacttcatccagcccaccgtgttcggagacgtcaaagatggcatgaccatcgccaaggaggagatcttcggaccagtgatgcagatcctcaaattcaagaccattgaggaggttgtggggcgagccaataattccaagtacgggctggctgccgctgtcttcacaaaggacctggacaaggccaattacctgtcccaagctctgcaggctgggactgtgtggatcaactgctacgatgtgtttggggcccagtccccatttggtggctataagatgtcggggagcggcagggagctgggcgagtatggcctgcaggcctacacggaagtgaagacggtcaccgtcaaagtgccacagaagaactcgtaaagtggcgtgcaggcttcctcagccagcgcccaaaaacccaacaagatcctgagaaaagccaccaccaagcacactgcgcctgccaagagaaaaccccttcaccaaagcgtcttgggccaagaaagtcaggatttgataaacagggcagggttggtgggcggtgtgtggggagcatcccagtaaactggggaagggaggagctctgtgcagactaccacgcgcacgcacacacgctcactgggtccttctgtgctggatgctggttccaccctcagtgcttaaacaaatgagcaataaa

SEQ ID NO: 109 - DNA, 21 bp, Artificial Sequence
  Description of Artificial Sequence Complement of human anti-ALDH2 ASO
agctgaaggggacaaggaaga

SEQ ID NO: 110 - DNA, 1989 bp, Homo sapiens
gctctcggtccgctcgctgtccgctagcccgctgcgatgttgcgcgctgccgccgctcgggccccgcctggccgccgcctcttgtcagccgccgccacccaggccgtgcctgcccccaaccagcagcccgaggtcttctgcaaccagattttcataaacaatgaatggcacgatgccgtcagcaggaaaacattccccaccgtcaatccgtccactggagaggtcatctgtcaggtagctgaaggggacaaggaagatgtggacaaggcacgtgaaggccgcccgggcgccttccagctgggctcaccttggcgccgcatggacgcatcacacagcggccggctgctgaaccgcctggccgatctgatcgagcgggaccggacctacctggcggccttggagaccctggacaatggcaagccctatgtcatctcctacctggtggatttggacatggtcctcaaatgtctccggtattatgccggctgggctgataagtaccacgggaaaaccatccccattgacggagacttcttcagctacacacgccatgaacctgtgggggtgtgcgggcagatcattccgtggaatttcccgctcctgatgcaagcatggaagctgggcccagccttggcaactggaaacgtggttgtgatgaaggtagctgagcagacacccctcaccgccctctatgtggccaacctgatcaaggaggctggctttccccctggtgtggtcaacattgtgcctggatttggccccacggctggggccgccattgcctcccatgaggatgtggacaaagtggcattcacaggctccactgagattggccgcgtaatccaggttgctgctgggagcagcaacctcaagagagtgaccttggagctgggggggaagagccccaacatcatcatgtcagatgccgatatggattgggccgtggaacaggcccacttcgccctgttcttcaaccagggccagtgctgctgtgccggctcccggaccttcgtgcaggaggacatctatgatgagtttgtggtgcggagcgttgcccgggccaagtctcgggtggtcgggaacccctttgatagcaagaccgagcaggggccgcaggtggatgaaactcagtttaagaagatcctcggctacatcaacacggggaagcaagagggggcgaagctgctgtgtggtgggggcattgctgctgaccgtggttacttcatccagcccactgtgtttggagatgtgcaggatggcatgaccatcgccaaggaggagatcttcgggccagtgatgcagatcctgaagttcaagaccatagaggaggttgttgggagagccaacaattccacgtacgggctggccgcagctgtcttcacaaaggatttggacaaggccaattacctgtcccaggccctccaggcgggcactgtgtgggtcaactgctatgatgtgtttggagcccagtcaccctttggtggctacaagatgtcggggagtggccgggagttgggcgagtacgggctgcaggcatacactgaagtgaaaactgtcacagtcaaagtgcctcagaagaactcataagaatcatgcaagcttcctccctcagccattgatggaaagttcagcaagatcagcaacaaaaccaagaaaaatgatccttgcgtgctgaatatctgaaaagagaaatttttcctacaaaatctcttgggtcaagaaagttctagaatttgaattgataaacatggtgggttggctgagggtaagagtatatgaggaaccttttaaacgacaacaatactgctagctttcaggatgatttttaaaaaatagattcaaatgtgttatcctctctctgaaacgcttcctataactcgagtttataggggaagaaaaagctattgtttacaattatatcaccattaaggcaactgctacaccctgctttgtattctgggctaagattcattaaaaactagctgctct

SEQ ID NO: 111 - DNA, 21 bp, Artificial Sequence
  Description of Artificial Sequence Human anti-ALDH2 ASO
tcttccttgtccccttcagct